(19) United States
(12) Patent Application Publication
(10) Pub. No.: US 2026/0078355 A1
(43) Pub. Date: Mar. 19, 2026

(54) RNA PROGRAMMABLE EPIGENETIC RNA MODIFIERS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Christopher Gerard Wilson, Cambridge, MA (US); Peter J. Chen, Cambridge, MA (US)

(73) Assignees: The Broad Institute, Inc., Cambridge, MA (US); President and Fellows of Harvard College, Cambridge, MA (US)

(21) Appl. No.: 19/402,431

(22) Filed: Nov. 26, 2025

Related U.S. Application Data
(63) Continuation of application No. 17/259,147, now Pat. No. 12,522,807.
(60) Provisional application No. 62/695,777, filed on Jul. 9, 2018.

(51) Int. Cl. C12N 9/10; A61K 38/00; C12N 9/02; C12N 9/22; C12N 15/11

(52) U.S. Cl. CPC C12N 9/1007; C12N 9/0071; C12N 9/22; C12N 15/11; C12Y 114/11; C12Y 201/01001; A61K 38/00; C07K 2319/00; C07K 2319/09; C07K 2319/095; C12N 2310/20

(57) ABSTRACT

The disclosure provides programmable methylation “writers” and demethylation “erasers” for editing the methylation state of RNA targets, e.g., an RNA transcriptome. In particular, the disclosure provides RNA methylation editor polynucleotide constructs and vectors comprising (i) an RNA programmable RNA binding domain (RNApRNAbd); and (ii) an effector domain, wherein the effector domain is capable of adding or removing a methyl group in an RNA. The disclosed RNA methylation editor constructs are capable of achieving limited off-target modifications in RNA molecules. Further, the disclosure provides methods for making and using the programmable methylation editors to modifying the methylation state of RNA. The disclosure further provides complexes comprising a methylation writer protein and a guide RNA molecule and complexes comprising a demethylation eraser protein and a guide RNA molecule. The disclosure further provides pharmaceutical compositions and cells comprising the disclosed fusion proteins and complexes.

### RELATED APPLICATIONS

This application is a continuation of and claims priority under 35 U.S.C. § 120 to U.S. patent application U.S. Ser. No. 17/259,147, filed Jan. 8, 2021, which is a national stage filing under 35 U.S.C. § 371 of International PCT application, PCT/US2019/041051, filed Jul. 9, 2019, which claims priority under 35 U.S.C. § 119(e) to U.S. Provisional application, U.S. Ser. No. 62/695,777, filed Jul. 9, 2018, and to U.S. Provisional application, U.S. Ser. No. 62/868,804, filed Jun. 28, 2019, the contents of each of which are incorporated by reference.

### STATEMENT REGARDING FEDERALLY SPONSORED RESEARCH

This invention was made with government support under Grant No. GM954507 awarded by the National Science Foundation. The government has certain rights in the invention.

### REFERENCE TO AN ELECTRONIC SEQUENCE LISTING

The contents of the electronic sequence listing (B119570055US03-SEQ-TNG.xml; Size: 76,695 bytes; and Date of Creation: Nov. 26, 2025) are herein incorporated by reference in their entirety.

### BACKGROUND

Epigenetics is the study of heritable changes in the genome that impact resulting phenotypes without involving actual changes to the underlying nucleotide sequences. These changes originate from a number of molecular mechanisms, including DNA methylation, histone modifications, and an ever expanding array of other epigenetic processes. Most recently, epigenetic changes have been determined to also encompass methylation states of RNA molecules. The underlying molecular mechanisms that impart epigenetic changes involving DNA and RNA and their roles in both normal (e.g., cell differentiation) and diseased cellular processes (e.g., cancer) are not fully understood.

While DNA is known to comprise linear chains of four nucleotides (A, G, C, and T), about a dozen naturally-occurring nucleotide variants are known (e.g., methylated nucleotides) which can have epigenetic effects. However, RNA comprises far more naturally-occurring nucleotide variants, i.e., about 140 alternative nucleotide forms, that can impact RNA structure, folding patterns, splicing, protein-binding properties of RNA molecules, and protein translation processes. The large size of RNA's nucleotide variant library to that of DNA's is not surprising since DNA is essentially unifunctional as a storage of genetic information, whereas RNA is engaged in a diverse set of structural, catalytic, and regulatory activities in cells and comprises a multitude of functionally and structurally distinct molecules (e.g., mRNA, rRNA, tRNA, miRNA, and others).

Internal RNA methylation modifications have recently gained importance as clinically significant epigenetic factors. For example, the most abundant internal modification of mRNA—N⁶-methyladenosine (m⁶A)—was found to accelerate pre-mRNA processing and mRNA transport in mammalian cells and is essential for mammals. Other known RNA epigenetic marks include pseudouridine (Ψ), N¹-methyladenosine (m¹A) and N6,20-O-dimethyladenosine (m⁶Am), as well as cytosine methylation to 5-methylcytosine and its oxidation product 5-hydroxymethylcytosine (hm⁵C). These marks are shown in FIG. 2A. While the precise function of these modification remain elusive, it has become evident that they have significant effects on mRNA stability, RNA folding, and ribosomal processing. For example, it is thought that the m⁶A modification has an effect on a plethora of cellular functions including stem cell proliferation and differentiation, cellular heat shock response, spermatogonia differentiation, maternal to zygotic transition, X-chromosome inactivation, UV DNA damage response, circadian clock function, and tumorigenesis. Aberrant m⁶A methylation has been implicated in diseases, including cancer.

Post-transcriptional methylation of adenine on the 6′ nitrogen (m⁶A) has come to the forefront as a functionally relevant mRNA modification, representing the most abundant modification within eukaryotic mRNAs ^{[5, 6]}. The m⁶A modification is primarily found in the 3′ UTR, 5′ UTR, in splice sites of mRNA transcripts, and within hairpins of microRNAs ^{[7, 8]} Different cell types display different m⁶A methylation patterns, hinting at a role in determining cellular differentiation. Interestingly, m⁶A has been found to be enzymatically eliminated from mRNAs, indicating that m⁶A is a dynamic modification like protein phosphorylation or DNA methylation ^{[5]}. Techniques detecting m⁶A methylation states of individual RNAs in the background of the transcriptome have recently been developed, allowing study of the effect of this modification on phenotype. MeRIP-Seq uses a combination of an m⁶A antibody and next generation sequencing to provide high resolution reads of m⁶A methylated RNA sites ^{[8]}. While the precise function of m⁶A modification remains elusive, it has become evident that it has significant effects on mRNA stability, RNA folding, and ribosomal processing. It is implicated in a plethora of cellular functions including stem cell proliferation and differentiation, cellular heat shock response, spermatogonia differentiation, maternal to zygotic transition, X-chromosome inactivation, UV DNA damage response, circadian clock function, and tumorigenesis ^{[7, 9-14]}. Aberrant m⁶A methylation has been implicated in diseases including cancer ^{[6, 15, 16]}.

Epigenetic marks, such as the m⁶A modification or other methylations, are introduced in RNA by enzymes and cofactors known as “writers.” The m⁶A writer is generally described as a large protein complex that includes three well-known components: METTL3, METTL14, and WTAP (i.e., the METTL3/METTL14/WTAP complex). The reverse process of RNA demethylation is performed by “erasers,” such as FTO (fat mass and obesity-associated protein) and ALKBH5 demethylases. Once RNA epigenetic modifications are laid down, they are recognized by specific “reader” proteins that bind to the modified nucleotides and mediate enhancement or inhibition of gene expression, i.e., determine the final outcome of the transcript. Unlike the writers and erasers, the readers primarily exist in the cytoplasm.

Currently, a need exists for specifically targeting the addition or removal of methylation sites in RNA. Tools capable of efficient and specific editing of RNA methylation sites would represent a significant advance in the art.

### SUMMARY OF THE DISCLOSURE

The present disclosure provides for fusion proteins comprising RNA programmable methylation “writers” and demethylation “erasers” for editing the methylation state of RNA targets that address this need in the art. The disclosed fusion proteins are surprisingly able to install modifications in reporter and endogenous mRNA transcripts in both nucleus and cytoplasm. These fusion proteins also provide for high RNA editing efficiency.

The (METTL3/METTL14) writer complex was recently identified as being responsible for targeted methylation of RNA ^{[17]}. The complex consists of a stable 184 kilodalton heterodimer consisting of two MTA-70 proteins, METTL3 and METTL14 ^{[17]}. METTL3 is an active s-adenosyl methionine (SAM) dependent methyltransferase which adds the methyl group from a SAM cofactor to the adenine in the sequence GGACU. METTL14 is homologous to METTL3, but comparison of the crystal structures within the heterodimer suggests that METTL14 is inactive, as the canonical SAM catalytic site is absent. METTL14 is most likely important for stabilizing METTL3 and RNA binding ^{[18]}. In the cell, the METTL3/14 core complex is localized to nuclear speckles and is regulated by a growing list of other binding partners, such as WTAP ^{[17]}. Two native erasers have also been identified, alkylated DNA repair protein ALKBH5 and FTO, both of which recognize the same GGm⁶aCU motif and serve to demethylate the adenine (see FIG. 4, top). Unlike the large writer complex, the erasers consist of small monomers. The erasers, like the writer complex, are also localized to the nucleus—specifically to nuclear speckles ^{[19]}. The third type of molecule, the “readers”, are a set of proteins that bind to m⁶A methylated regions of RNA and determine the final outcome of the transcript. Unlike the writers and erasers, these readers primarily exist in the cytoplasm (see FIG. 4, lower).

Recently, several programmable editors of DNA and RNA have been constructed by tethering nucleic acid-modifying enzymes to inactivated Cas9 or the RNA targeting homolog Cas13b ^{[20-22]}. This approach combines the flexibility of Cas9 targeting with specific nucleic acid modifying enzymes. Examples include deamination of cytosine resulting in a C→T mutation ^{[20]}, A→G mutations ^{[21]}, and DNA methylation ^{[23]}. The recent discovery of a family of Cas enzymes that target RNA instead of DNA has allowed for a similar approach on RNA. Tethering of the RNA-modifying enzyme ADAR to Cas13b resulted in a mRNA targetable complex capable of single base editing (A→G) ^{[22]}.

In particular, the disclosure provides RNA methylation editor constructs comprising (i) an RNA programmable RNA binding domain (RNApRNAbd); and (ii) an effector domain, wherein the effector domain is capable of adding or removing a methyl group in an RNA. In other words, the effector domain can in some embodiments be an RNA methylation writer, such as dCas13b-METTL3. In other embodiments, the effector domain can be an RNA methylation eraser, such as dCas13b-ALKBH5. In addition, the present disclosure provides for nucleic acid molecules encoding and/or expressing the RNA methylation editors as described herein, as well as expression vectors for expressing the RNA methylation editors described herein, host cells comprising said nucleic acid molecules and expression vectors, and compositions for delivering and/or administering nucleic acid-based embodiments described herein. In addition, the disclosure provides for isolated RNA methylation editors, as well as compositions comprising said isolated RNA methylation editors as described herein. Still further, the present disclosure provides for methods of making the RNA methylation editors, as well as methods of using the RNA methylation editors or nucleic acid molecules encoding the RNA methylation editors in applications including editing, modifying, or otherwise altering the methylation state of a target RNA molecule in a specific and/or targeted manner, i.e., by minimizing changes to the methylation status of off-target loci.

The disclosure also provides methods for efficiently and specifically editing the methylation state of a target RNA molecule with a RNA methylation editor described herein (e.g., in the form of an isolated RNA methylation editor as described herein or a vector encoding same) and conducting methylation state editing of target RNA molecule in a specific manner and without introducing off-site changes in methylation states. Still further, the disclosure provides therapeutic methods for treating a disease and/or for altering or changing a trait or condition associated with an epigenetic state (e.g., methylation state) by contacting a target RNA molecule with an RNA methylation editor (e.g., in the form of an isolated RNA methylation editor or a vector encoding same) and conducting methylation editing to treat the disease or phenotype associated with the epigenetic condition, without actually making any changes in the nucleotide sequence of the target RNA.

Thus, in one aspect, the disclosure provides a fusion protein that comprises an RNA programmable RNA binding domain (RNApRNAbd) and an effector domain, wherein the effector domain is capable of adding or removing a methyl group in an RNA.

The effector domain can be a methyltransferase, such as, METTL3 or METTL14, or METTL3/METTL14 fusion, or M.EcoGII, TrmI, Trmt61B, Trm4, Dnm2, or RlmI. The effector domain that is used in the disclosure can have various activities, including making an N⁶-methyladenosine (m⁶A) modification in the RNA, making a 1-methyladenosine (m¹A) modification in the RNA, or making a 5-hydroxymethylcytidine (m⁵C) modification in the RNA.

In various embodiments, the effector domain can have an amino acid sequence that is at least 80%, 85%, 90%, 95%, 98%, or 99% identical to the amino acid sequence of any one of SEQ ID NOs: 3 (Q86U44 (METTL3)), SEQ ID NO: 4 (Q9HCE5 (METTL14)), SEQ ID NO: 5 (EGR75201 (M.EcoGII)), SEQ ID NO: 6 (P9WFZ0 (TrmI)), SEQ ID NO: 7 (Q9BVS5 (Trmt61B)), SEQ ID NO: 8 (Q08J23 (Trm4)), SEQ ID NO: 9 (O14717 (Dnmt2)), or SEQ ID NO: 10 (P75876 (RlmI)).

In various other embodiments, the effector domain can be a demethylase, such as, but not limited to, ALKBH5 or FTO. In addition, the demethylases contemplated herein can be an amino acid sequence that is at least 80%, 85%, 90%, 95%, 98%, or 99% identical to the amino acid sequence of any one of SEQ ID NO: 11 (Q6P6C2 (ALKBH5)) or SEQ ID NO: 12 (Q9C0B1 (FTO)).

The RNA methylation editors can provide editing for any type of RNA molecule target, including mRNA, tRNA, or rRNA molecules. In particular embodiments, the RNA target is an endogenous target sequence within a transcriptome, e.g., a mammalian transcriptome. In certain embodiments, the RNA target is a target sequence in a human transcriptome. In other embodiments, the RNA target is a reporter transcript.

In particular embodiments, the RNA target may be a beta-actin (ACTB) mRNA, adenosine at locus 1216 (A1216) or a glyceraldehyde 3-phosphate dehydrogenase (GAPDH), adenosine at locus 673 (A673).

In various embodiments, the RNA methylation editor fusion proteins can modulate the methylation state of a target RNA sequence. The target RNA sequence may comprise a mRNA, tRNA, rRNA, microRNA, siRNA, or any other type of expressed cellular RNA of a cell and which is encoded by a cell genome of an organism. The organism may be any type, including prokaryotes, eukaryotes, plants, bacteria, vertebrates, mammals, humans, and animals or pets. The target RNA sequence may comprise a sequence in the transcriptome of an organism. The target RNA sequence may comprise a transcript of a genomic DNA sequence.

In various embodiments, the disclosed fusion proteins install modifications in target RNA molecules in the cytoplasm of the target cell, the nucleus of the target cell, or both. In various embodiments, the disclosed fusion proteins install modifications with high RNA editing efficiencies (i.e., low off-target modification frequencies) in target RNA molecules in the cytoplasm of the target cell, the nucleus of the target cell, or both.

In other aspects, the disclosure provides methods of using the fusion editing polynucleotide constructs described herein. In one embodiment, the disclosure embraces a method of contacting an RNA molecule with any fusion protein described herein. In various embodiments, the RNA molecule that is contacted is associated with a disease or disorder. The activity of the fusion protein can result in the addition of a methyl group to the RNA molecule, or the removal of a methyl group from the RNA molecule. Specific modifications to the target RNA molecule by the fusion proteins can include an N⁶-methyladenosine (m⁶A) modification in the RNA molecule, a 1-methyladenosine (m¹A) modification in the RNA molecule, or a 5-hydroxymethylcytidine (m⁵C) modification in the RNA molecule. In one embodiment, the fusion protein results in the removal of an N⁶-methyladenosine (m⁶A) modification in the RNA molecule, or the removal of a 1-methyladenosine (m¹A) modification in the RNA molecule, or the removal of a 5-hydroxymethylcytidine (m⁵C) modification in the RNA molecule.

Such changes in the RNA molecule can result in various changed characteristics, including an increase in stability of the RNA molecule, an increase in expression of the RNA molecule, a decrease in stability of the RNA molecule, or a decrease in expression of the RNA molecule. In some embodiments, the RNA molecule is a pre-mRNA, and the contacting results in a splicing modification of the pre-mRNA. The splicing modification can comprise splicing out an exon, or preventing the splicing out of an exon. The contacting can also result in a change of the isoform of the mRNA molecule.

The target RNA molecules changed by the fusion proteins disclosed herein can be performed in vivo in a subject, in vitro, or ex vivo. The subject can have a disease or a disorder that is associated with a particular state of RNA methylation.

In other embodiments, the disclosure provides polynucleotide sequences which encode the fusion proteins described herein, and to vectors comprising the polynucleotide sequences. The vectors can comprise a heterologous promoter driving expression of the polynucleotide.

In other embodiments, the disclosure provides pharmaceutical compositions that comprise the fusion proteins described herein, and a pharmaceutically acceptable carrier.

In other aspects, the disclosure also relates to kits comprising a nucleic acid construct, comprising (a) a nucleic acid sequence encoding a fusion protein described herein, and (b) a heterologous promoter that drives expression of the sequence of (a). The kits may further comprise an expression construct encoding a guide RNA backbone, wherein the construct comprises a cloning site positioned to allow the cloning of a nucleic acid sequence identical or complementary to a target sequence into the guide RNA backbone.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

FIG. 1 shows a schematic representation of DNA base editing and RNA base editing. RNA base editing complements DNA base editing, is transient rather than permanent, and is not subject to the same PAM requirements as with DNA base editing.

FIGS. 2A-2D show schematic representations of examples of known cellular post-transcriptional RNA modifications. In FIG. 2A, a typical mRNA molecule is represented comprising a 5′ cap structure and a poly(A) tail, and a coding region (thick darkened line) and 5′ end and 3′ end non-coding regions (thin darkened lines). Various positions along the mRNA molecule are marked with common RNA modifications, including (beginning from the 5′ end) a 2′-O-methylated nucleotide (“Nm”), 5-methyl cytosine (“m⁵C”), N1-methyladenosine (“m¹A”), pseudouridine (“Ψ”), 5-hydroxymethylcytosine (“hm⁵C”), and N⁶,2′-O-dimethyladenosine (“m⁶A”). These modifications have an epigenetic effect on various cellular processes, including altered stability or localization (FIG. 2B), modified splicing (FIG. 2C), and expression changes or isoform switching (FIG. 2D). FIG. 2A is reproduced from Roundtree et al., “Dynamic RNA Modifications in Gene Expression Regulation,” Cell, 169, Jun. 15, 2017, pp. 1187-1200 (which is incorporated herein by reference).

FIG. 3 is a schematic of a known construct that fused a Type VI CRISPR-Cas programmable single-effector RNA-guided ribonuclease Cas13 (catalytically inactive variant, dCas13) to ADAR2 (adenosine deaminase acting on RNA type 2). See Cox et al., “RNA editing with CRISPR-Cas13,” Science. 2017 Nov. 24; 358(6366):1019-1027 (incorporated herein by reference). This system, referred to as RNA Editing for Programmable A to I Replacement (REPAIR), which has no strict sequence constraints, can be used to edit full-length transcripts containing pathogenic mutations. REPAIR presents a RNA-editing platform with broad applicability for research, therapeutics, and biotechnology.

FIG. 4 provides a schematic showing the methylation of the 6′ nitrogen in adenine in RNA by a METTL3/METTL14/WTAP “writer” complex forming N⁶-methyladenine (m⁶A). The demethylation reaction can be carried out by an “eraser” or demethylase, e.g., ALKBH5 or FTO demethylases, to reform adenine. As shown in the lower section, the location or positioning of the m⁶A on the target RNA determines which particular “reader” protein gains access, leading to a variety of RNA processing outcomes. As shown, readers YTHDF2 in the 5′ UTR (enhanced translation), HNRNPC at a splice site (alternative splicing), YTHDF1 in the 3′ UTR (mRNA decay), or YTHDF2 in the 3′ UTR (enhanced translation) lead to various processing outcomes. Such modifications may enhance translation, modulate alternative splicing, or modulate mRNA decay.

FIG. 5 provides a schematic showing that the engineered sequence-programmable erasers (e.g., dCas13b-ALKBH5) and sequence-programmable writers (e.g., dCas13b-METTL3/14/WTAP) described herein would operate in the cytoplasm following release of target mRNAs from the nucleus (which will have naturally received their m⁶A modifications therein). The “reprogrammed” mRNAs would then be “read” by cytoplasmic “readers” that determine the fate of the RNA transcript (e.g., YTHD2).

FIG. 6 demonstrates the effect of METTL3/METTL14 complex on the binding to RNA targets is improved for the complex relative to METTL3 alone. However, METTL3 may be the ideal fusion to Cas13b, as the increase in local concentration provided by the Cas13b would overcome the weak Km of METTL3 alone and provide guide RNA-dependent specificity for the target RNA.

FIG. 7 shows a schematic representation of cellular RNA methylation assays in which total cellular RNA is contacted with immobilized m⁶A antibody, which enriches for methylated RNA. Subsequent RT-qPCR or RNA-seq is performed to characterize bound RNAs.

FIG. 8 shows exemplary data regarding guide RNA- and METTL-dependent methylation of RNA in E. coli. The data demonstrate that RNA methylation depends on Cas13B, METTL3 activity, and guide RNA.

FIG. 9 shows a schematic representation of experiments characterizing off-target RNA methylation. Exemplary data demonstrate modest off-target activity in E. coli.

FIG. 10 shows a schematic representation of a strategy for m⁶A editing in mammalian cells. Plasmids encoding RNA-modifying proteins and a guide RNA are transfected into mammalian HEK293T cells with a target RNA. The m⁶A-mediated increase of translation efficiency of the target RNA is measured by MeRIP-seq. Here, the target RNA is Cypridina luciferase coding sequence (CLuc CDS).

FIGS. 11A-11C provide a schematic depicting (FIG. 11A) a recombinant E. coli containing two vectors as described in the Examples; (FIG. 11B) Vector 1 expressing Cas13b fused to METTL3 under a T7 promoter inducible by Isopropyl β-D-1-thiogalactopyranoside (IPTG) and the guide RNA (crRNA) with protospacer (purple) under a constitutive promoter; (FIG. 11C) Vector 2 expressing the target RNA substrate with target sequence (purple) surrounded by canonical METTL3 GGACU recognition sites of methylation.

FIG. 12 depicts model of aggressive tumor formation based on demethylated A Disintegrin And Metalloproteinase 19 (ADAM19) target. The transcript may be targeted by a fusion protein described herein to restore the normal state of ADAM19 m⁶A methylation to avoid the aggressive tumor formation condition.

FIG. 13A shows METTL3 (M3) and METTL14 (M14) are homologous m⁶A methyltransferases that constitute the core writing complex. Additional components of the tetradimeric M3/M14 “writer” influence the formation and activity of this core complex. This S-adenosyl methionine (SAM)-dependent complex catalyzes the methylation of the 6′ nitrogen of adenine in mRNA. FTO and AlkBH5 have been identified as responsible for removal of the of the methyl group on the 6′ nitrogen of m⁶A, and are thus characterized as “erasers”. Readers recognize the m⁶A mark at specific locations on the RNA and direct it to various outcomes, including transcript degradation, enhanced translation and alternative splicing. FIG. 13B shows the M3/M14 core writing unit and its accessory proteins methylate transcribed mRNA in the nucleus. NLS-m⁶A writers can add further methylation groups in the cytoplasm and nucleus which are then read by cytoplasmic readers in the cytoplasm or nucleic readers in the nucleus.

FIGS. 14A-14D depict enzyme activity screening in E. coli. FIG. 14A is a schematic representation of the dCas13b-M3 and dCas13b-M3M14 editors as linearized constructs. FIG. 14B shows the transformation into E. coli of two plasmid vectors, one containing constitutively-expressed gRNA and an IPTG-inducible m⁶A-editor and the other containing a synthetic target transcript containing m⁶A methylation sites (GGACU) arrayed around a gRNA-targeting sequence FIG. 14C shows meRIP-RT-qPCR quantification of methylation events under induced conditions, non-induced conditions and induced without the gRNA. Values are relative to a synthetic target-only control (denoted by the dotted line). FIG. 14D is a Venn Diagram depicting writer off-targeting in the bacterial background using meRIP-seq.

FIGS. 15A-15D depict enzyme activity screening in mammalian cells. FIG. 15A shows the experimental setup for screening includes three plasmid vectors, one containing the writer, one containing a guide RNA, and one containing a target transcript. Enrichment of m⁶A in target molecules was measured using meRIP-RT-qPCR. FIG. 15B shows the target molecules. The top represents a synthetic 3′ UTR comprising m⁶A methylation sites arrayed around a gRNA-targeting sequence fused to Cluc luminescence reporter. The bottom represents the target comprising a suppressor of cytokine signaling 2 gene (SOCS2) 3′ UTR fused to a Cluc reporter. FIG. 15C shows meRIP-RT-qPCR results generated by editor activity targeting the synthetic target (“Cluc-syn”). FIG. 15D shows meRIP-RT-qPCR results generated by editor activity targeting the SOCS2 3′ UTR.

FIGS. 16A-16D depict comparison plots and rank plots that quantify the results of an off-targeting screen in mammalian cells. FIG. 16A shows a comparison plot of % Methylation between the methyltransferase-inactive dCas13b-dM3M14 and targeted, methyltransferase-active dCas13b-M3M14. The Cluc-SOCS2 fusion target is shown in red. FIG. 16B shows a comparison plot of % Methylation between catalytically inactive dCas13b-dM3M14 and non-targeted active NT-dCas13b-M3M14. The Cluc-SOCS2 target is shown in red. FIG. 16C shows a rank plot of the ratio of dCas13b-dM3M14 and targeted active dCas13b-M3M14. The Cluc-SOCS2 target is shown as a darkened dot in the rank portion and as a darkened bar in the density plot. FIG. 16D shows a rank plot of ratio dCas13b-dM3M14 and non-targeted active NT-dCas13b-M3M14.

FIGS. 17A-17B shows cellular localization of the dCas13b editors. FIG. 17A is a schematic representation of all variants of the m⁶A editor constructs (linearized). NES: nuclear export sequence; NLS: nuclear localization sequence. FIG. 17B shows immunofluorescence images of the 3× hemagglutinin (HA)-tagged dCas13b m⁶A editors. A beta actin (ACTB) gRNA was co-transfected with the editors. Darker shading: DAPI; Lighter shading: HA tag.

FIGS. 18A-18B show MeRIP-RTqPCR results of methylation frequencies in another endogenous transcript target, Glyceraldehyde 3-phosphate dehydrogenase (GAPDH) at A673, which is unmethylated in HEK293T cells. FIG. 18A shows meRIP-RT-qPCR results generated by editor activity targeting the synthetic RNA target. FIG. 18B shows meRIP-RT-qPCR results generated by editor activity targeting the SOCS2 3′ UTR.

FIGS. 19A-19D are differential RNA-seq Volcano plots showing the differential expression of transcripts by the four active editors as compared to catalytically dead versions thereof. Darkened dots indicate transcripts with significant changes in expression. The numbers in the upper right corners indicate the quantity of transcripts with a significant change in expression.

FIGS. 20A-20C show the evaluation of editing using various CLuc coding sequence (CDS) guides and nucleus-localized and cytoplasm-localized writers. FIG. 20A shows five guides for CLuc. FIG. 20B shows two guides for HSPA1A 5′ UTR-Cluc and HSPH15 5′ UTR-Cluc. FIG. 20C shows two guides for Cluc-syn 3′ UTR, Cluc-SOCS2 3′ UTR, and Cluc-NANOG 3′ UTR. Cluc reporters were targeted with NES dCas13 and NLSdCas13 at the CDS (within Cluc coding region), 5′ UTR (from endogenous transcripts placed at the end of Cluc), and 3′ UTR (from endogenous transcripts placed at the other end of Cluc). The RNA abundance and protein expression (luciferase signal) of these Cluc reporters were measured and normalized to a Gaussia luciferase (Gluc) dosing control.

FIG. 21A shows the modification site for A1216 in ACTB mRNA and a graph showing the normalized ACTB m⁶A enrichment for plasmid vectors and METTL3. Data for both guides and non-targeting guides are shown. FIG. 21B shows Normalized ACTB m⁶A enrichment for various methyltransferases. Data for both guides and non-targeting guides are shown.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

A type VI CRISPR-associated RNA-guided RNase enzyme reported in Abudayyeh O O, et al. “C2c2 is a single-component programmable RNA-guided RNA-targeting CRISPR effector,” Science. 2016; 353 and further discussed in Shmakov S, et al. Discovery and Functional Characterization of Diverse Class 2 CRISPR-Cas Systems. Mol Cell. 2015; 60:385-397, Shmakov S, et al. Diversity and evolution of class 2 CRISPR-Cas systems. Nat Rev Microbiol. 2017; 15:169-182, and Smargon A A, et al. Cas13b Is a Type VI-B CRISPR-Associated RNA-Guided RNase Differentially Regulated by Accessory Proteins Csx27 and Csx28. Mol Cell. 2017; 65:618-630 e617 (each of which are incorporated herein by reference). Cas13 enzymes have two Higher Eukaryotes and Prokaryotes Nucleotide-binding (HEPN) endoRNase domains that mediate precise RNA cleavage with a preference for targets with protospacer flanking site (PFS) motif observed biochemically and in bacteria (10, 11). Three Cas13 protein families have been identified to date: Cas13a (previously known as C2c2), Cas13b, Cas13c (Smargon A A et al., Mol Cell. 2017 Feb. 16; 65(4):618-630, incorporated herein by reference), and most recently, Cas13d (W. X. Yan, “Cas13d is a compact RNA-targeting Type VI CRISPR effector positively modulated by a WYL-domain-containing accessory protein,” Molecular Cell, Apr. 19, 2018, Vol. 70, pp. 327-339, which is incorporated herein by reference). “dCas13” refers to a variant of Cas13 which catalytically dead, i.e., has no endoRNase activity due to mutations in conserved regions of the HEPN domains as reported in Cox et al. Similarly, “dCas13a, dCas13b, dCas13c, and dCas13d” refer to the corresponding catalytically dead variants.

As used herein, Cas13b is a Cas13 subtype. In some embodiments, the Cas13b is derived from Prevotella sp. P5-125. In some embodiments, the Cas13b is a truncated variant of the Cas13b of Prevotella sp. P5-125. In one embodiment, Cas13b has the following amino acid sequence: MNIPALVENQKKYFGTYSVMAMLNAQTVLDHIQKVADIEGEQNENNENLWFHPVMSHL YNAKNGYDKQPEKTMFIIERLQSYFPFLKIMAENQREYSNGKYKQNRVEVNSNDIFEVL KRAFGVLKMYRDLTNAYKTYEEKLNDGCEFLTSTEQPLSGMINNYYTVALRNMNERYG YKTEDLAFIQDKRFKFVKDAYGKKKSQVNTGFFLSLQDYNGDTQKKLHLSGVGIALLIC LFLDKQYINIFLSRLPIFSSYNAQSEERRIIIRSFGINSIKLPKDRIHSEKSNKSVAMDMLNE VKRCPDELFTTLSAEKQSRFRIISDDHNEVLMKRSSDRFVPLLLQYIDYGKLFDHIRFHVN MGKLRYLLKADKTCIDGQTRVRVIEQPLNGFGRLEEAETMRKQENGTFGNSGIRIRDFEN MKRDDANPANYPYIVDTYTHYILENNKVEMFINDKEDSAPLLPVIEDDRYVVKTIPSCR MSTLEIPAMAFHMFLFGSKKTEKLIVDVHNRYKRLFQAMQKEEVTAENIASFGIAESDLP QKILDLISGNAHGKDVDAFIRLTVDDMLTDTERRIKRFKDDRKSIRSADNKMGKRGFKQI STGKLADFLAKDIVLFQPSVNDGENKITGLNYRIMQSAIAVYDSGDDYEAKQQFKLMFE KARLIGKGTTEPHPFLYKVFARSIPANAVEFYERYLIERKFYLTGLSNEIKKGNRVDVPFIR RDQNKWKTPAMKTLGRIYSEDLPVELPRQMFDNEIKSHLKSLPQMEGIDENNANVTYLI AEYMKRVLDDDFQTFYQWNRNYRYMDMLKGEYDRKGSLQHCFTSVEEREGLWKERA SRTERYRKQASNKIRSNRQMRNASSEEIETILDKRLSNSRNEYQKSEKVIRRYRVQDALLF LLAKKTLTELADFDGERFKLKEIMPDAEKGILSEIMPMSFTFEKGGKKYTITSEGMKLKN YGDFFVLASDKRIGNLLELVGSDIVSKED (SEQ ID NO: 1). The disclosure embraces the use of Cas13b homologs, fragments, and functional variants thereof, including polypeptides having at least 80%, 85%, 90%, 95%, 96%, 97%, 98%, or 99% sequence identity with SEQ ID NO: 1 and which can be derived or obtained from any organism or species. Preferably, the Cas13b homologs, fragments, and functional variants lack or substantially lack nuclease activity but retain the ability to bind to RNA, i.e., “dCas13b.”

As used herein, Cas13d is a Cas13 subtype. In some embodiments, the Cas13d is derived from Ruminococcus flavefaciens. In some embodiments, the Cas13d is a truncated variant of the Cas13d of Ruminococcus flavefaciens. In one embodiment, Cas13d has the following amino acid sequence: MIEKKKSFAKGMGVKSTLVSGSKVYMTTFAEGSDARLEKIVEGDSIRSVNEGEAFSAEM ADKNAGYKIGNAKFSHPKGYAVVANNPLYTGPVQQDMLGLKETLEKRYFGESADGNDN ICIQVIHNILDIEKILAEYITNAAYAVNNISGLDKDIIGFGKFSTVYTYDEFKDPEHHRAAFN NNDKLINAIKAQYDEFDNFLDNPRLGYFGQAFFSKEGRNYIINYGNECYDILALLSGLAH WVVANNEEESRISRTWLYNLDKNLDNEYISTLNYLYDRITNELTNSFSKNSAANVNYIAE TLGINPAEFAEQYFRFSIMKEQKNLGFNITKLREVMLDRKDMSEIRKNHKVFDSIRTKVY TMMDFVIYRYYIEEDAKVAAANKSLPDNEKSLSEKDIFVINLRGSFNDDQKDALYYDEA NRIWRKLENIMHNIKEFRGNKTREYKKKDAPRLPRILPAGRDVSAFSKLMYALTMFLDG KEINDLLTTLINKFDNIQSFLKVMPLIGVNAKFVEEYAFFKDSAKIADELRLIKSFARMGEP IADARRAMYIDAIRILGTNLSYDELKALADTFSLDENGNKLKKGKHGMRNFIINNVISNK RFHYLIRYGDPAHLHEIAKNEAVVKFVLGRIADIQKKQGQNGKNQIDRYYETCIGKDKG KSVSEKVDALTKIITGMNYDQFDKKRSVIEDTGRENAEREKFKKIISLYLTVIYHILKNIVN INARYVIGFHCVERDAQLYKEKGYDINLKKLEEKGFSSVTKLCAGIDETAPDKRKDVEKE MAERAKESIDSLESANPKLYANYIKYSDEKKAEEFTRQINREKAKTALNAYLRNTKWNVI IREDLLRIDNKTCTLFANKAVALEVARYVHAYINDIAEVNSYFQLYHYIMQRIIMNERYEK SSGKVSEYFDAVNDEKKYNDRLLKLLCVPFGYCIPRFKNLSIEALFDRNEAAKFDKEKK KVSGNS (SEQ ID NO: 2). The disclosure embraces the use of Cas13d homologs, fragments, and functional variants thereof, including polypeptides having at least 80%, 85%, 90%, 95%, 96%, 97%, 98%, or 99% sequence identity with SEQ ID NO: 2 and isolated or obtained from any organism or species. Preferably, the Cas13d homologs, fragments, and functional variants lack or substantially lack a nuclease activity but retain the ability to bind to RNA, i.e., “dCas13d.”

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821 (2012), the entire contents of which is herein incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., Proc. Natl. Acad. Sci. U.S.A. 98:4658-4663 (2001); “CRISPR RNA maturation by trans-encoded small RNA and host factor RNase III.” Deltcheva E., et al. Nature 471:602-607 (2011); and “A programmable dual-RNA-guided DNA endonuclease in adaptive bacterial immunity.” Jinek M., et al. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:(5): 726-737; the entire contents of which are incorporated herein by reference.

As used herein, the “effector domain” refers to a polypeptide that is capable of enzymatically modifying an epigenetic feature of a cell, e.g., a methylation state of a DNA or RNA molecule. For example, an effector domain can include a polypeptide that is capable of adding or removing a methyl group in an RNA (i.e., a methyltransferase or “writer” domain or a demethylase or “eraser” domain). The RNA can be any type, including a messenger RNA (“mRNA”), a transfer RNA (“tRNA”), a ribosomal RNA (rRNA), a small nuclear RNA (“snRNA”), an antisense RNA (“asRNA”), long noncoding RNA (“lncRNA”), small interfering RNA (“siRNA”), and short hairpin RNA (“shRNA”). The effector domain can include a methyltransferase (or “writer” domain), such as, but not limited to, METTLE3 (e.g., UNIPROT Accession No. Q86U44), METTL14 (e.g., UNIPROT Accession No. Q9HCE5), M.EcoGII (e.g., GenBank Accession No. EGR75201), TrmI (e.g., UNIPROT Accession No. P9WFZ0), Trmt61B (e.g., UNIPROT Accession No. Q9BVS5), Trm4 (e.g., UNIPROT Accession No. Q08J23), Dnmt2 (e.g., UNIPROT Accession No. O14717), and RlmI (e.g., UNIPROT Accession No. P75876). The effector domain can also include a demethylase (or “eraser” domain), such as, but not limited to, ALKBH5 or FTO (fat mass and obesity-associated protein). Collectively, the methylation “writers” and the demethylation “erasers” can be referred to as “RNA methylation editors” or “RNA methylation editor constructs” or the like.

The term “effective amount,” as used herein, refers to an amount of a biologically active agent that is sufficient to elicit a desired biological response. For example, in some embodiments, an effective amount of a RNA methylation editor may refer to the amount of the editor that is sufficient to edit a target site methylation state. In some embodiments, an effective amount of an editor provided herein, e.g., of a fusion protein comprising a RNA-programmable RNA binding protein and an effector domain, may refer to the amount of the fusion protein that is sufficient to induce editing of a target site specifically bound and edited by the fusion protein. As will be appreciated by the skilled artisan, the effective amount of an agent, e.g., a fusion protein, a methyltransferase, a demethylase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

As used herein, the term “isolated protein” or “isolated nucleic acid” refers to a protein or nucleic acid that by virtue of its origin or source of derivation is not associated with naturally associated components that accompany it in its native state; is substantially free of other proteins or nucleic acids from the same species; is expressed by a cell from a different species; or does not occur in nature. Thus, a polypeptide or nucleic acid that is chemically synthesized or synthesized in a cellular system different from the cell from which it naturally originates will be “isolated” from its naturally associated components. A protein or nucleic acid may also be rendered substantially free of naturally associated components by isolation, using protein purification techniques well known in the art.

The term “linker,” as used herein, refers to a chemical group or a molecule linking two molecules or moieties, e.g., the linkage of an RNA programmable RNA binding domain and a methyltransferase domain or demethylase domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The terms “methylation site,” “methylation location” and “methylation locus” are synonymous and refer to a nucleobase loci in an mRNA molecule (e.g. adenosine nucleobases) that has variable methylation states that may be recognized by a methyltransferase enzyme or a demethylase enzyme.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions,

The terms “non-naturally occurring” or “engineered” are used interchangeably and indicate the involvement of the hand of wo/man. The terms, when referring to nucleic acid molecules or polypeptides (e.g., Cas13) mean that the nucleic acid molecule or the polypeptide is at least substantially free from at least one other component with which they are naturally associated in nature and/or as found in nature (e.g., an amino acid sequence not found in nature).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues.

The term “guide sequence” refers the one or more nucleic acid molecules which associate with and direct or otherwise program a RNA programmable RNA binding domain to localize to a specific target RNA sequence (e.g., a mRNA) that is complementary to the one or more guide RNAs (or a portion or region thereof) associated with the RNA programmable RNA binding domain, thereby causing the RNA programmable RNA binding to bind to the target RNA at the specific target site. A non-limiting example is a guide RNA of a Cas13 protein of a CRISPR-Cas13 RNA editing system. Exemplary guide sequences are disclosed in Table 2.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which is an amino acid sequence that tags, designates, or otherwise marks a protein for export out of the nucleus by nuclear transport. Thus, a single nuclear localization signal can direct the entity with which it is associated to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

A nuclear export sequence (NES) can be of any size and composition, for example more than 25, 25, 15, 12, 10, 8, 7, 6, 5 or 4 amino acids, but will preferably comprise at least a four to ten amino acid sequence known to function as a nuclear export signal.

As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the terms “off-target modification frequency,” “off-target insertion rate” and “off-target methylation rate” refer to the degree of methylation at unintended target sites, i.e. at nucleobases other than the target nucleobase sequence, in the target RNA molecule. This degree of methylation may be quantified by evaluating the number of methylation events at each possible methylation site other than the target site (or locus) in the RNA molecule, and dividing by the number of possible methylation sites (or loci). For example, a fusion protein activity that produces 370 methylation events at off-target loci out of 37,000 possible methylation loci results in an off-target modification frequency of 1.0%. Off-target modification frequencies may be measured in any target RNA molecule, including endogenous and reporter transcripts. The presence of a methylation event may be determined by high-throughput screening of sequencing reads of the target RNA molecule, e.g. through the MeRIP-seq and differential RNA-seq techniques, or by other methods known in the art.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas13 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

As used herein, the term “RNA modification” refers to any post-translational modification of an RNA sequence. This includes, but is not limited to, 2′-O-methylated nucleotide (“Nm”), 5-methyl cytosine (“m⁵C”), N1-methyladenosine (“m¹A”), pseudouridine (“Ψ”), 5-hydroxymethylcytosine (“hm⁵C”), and N⁶,2′-O-dimethyladenosine (“m⁶A”).

The term “RNA editing efficiency,” as used herein, refers to the number or proportion of intended RNA loci that are edited. For example, if an editor edits 10% of the RNA nucleobases that it is intended to target (e.g., within a cell or within a population of cells), then the editor can be described as being 10% efficient. Some aspects of RNA editing efficiency embrace the modification (e.g. methylation) of a specific nucleobase.

RNA editing efficiency may also be expressed in terms of generating low off-target editing (or modification) frequencies. It is generally accepted that generating an off-target modification frequency of 5% or less (as measured over total target loci) is high editing efficiency. As with determination of off-target modification frequencies, RNA editing efficiency may be determined by high-throughput screening of sequencing reads of the target RNA molecule, e.g. through the MeRIP-seq and differential RNA-seq techniques.

The term “RNA-programmable RNA binding domain” refers to a polypeptide that forms a complex with (e.g., binds or associates with) one or more protein-associating guide RNA molecules which guide the binding protein to target an RNA molecule (e.g., a mRNA, rRNA, or tRNA molecule) having a sequence that is complementary to the one or more protein-associating guide RNA molecules. This concept embraces CRISPR/Cas proteins that have been modified or adapted to target RNA instead of DNA (e.g., spCas9 system), as well as native or naturally occurring RNA-targeting CRISPR/Cas protein (e.g., Cas13, including Cas13a, Cas13b, Cas13c, and Cas13d), and any homologs and derivatives thereof (e.g., nuclease-deficient variants) isolated or obtained from any organism or species. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules.

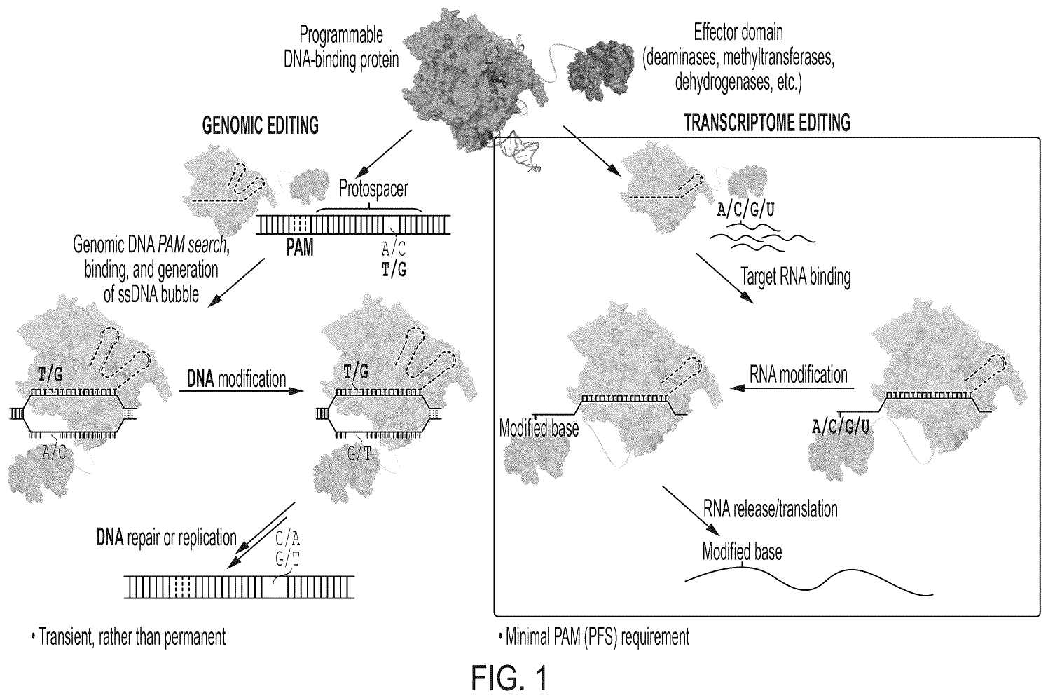

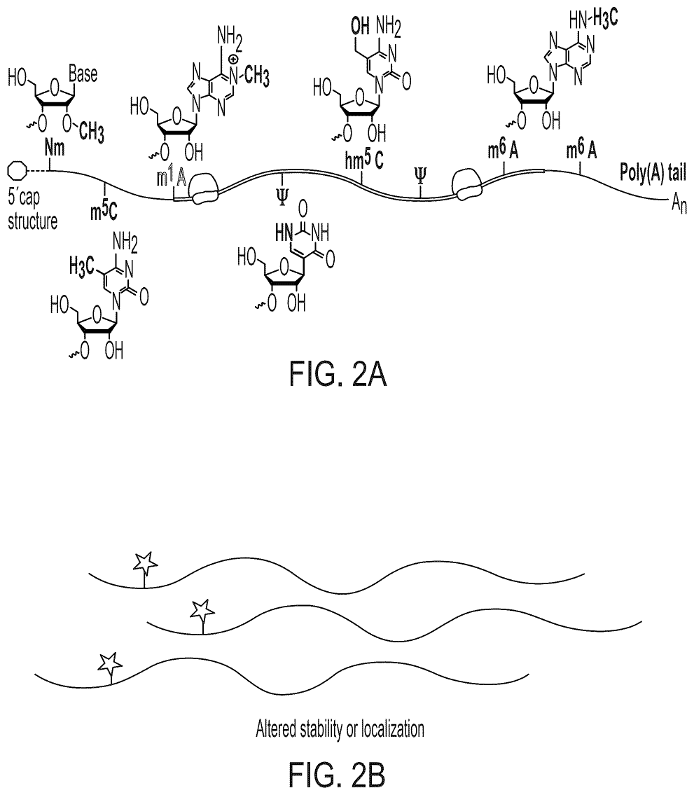

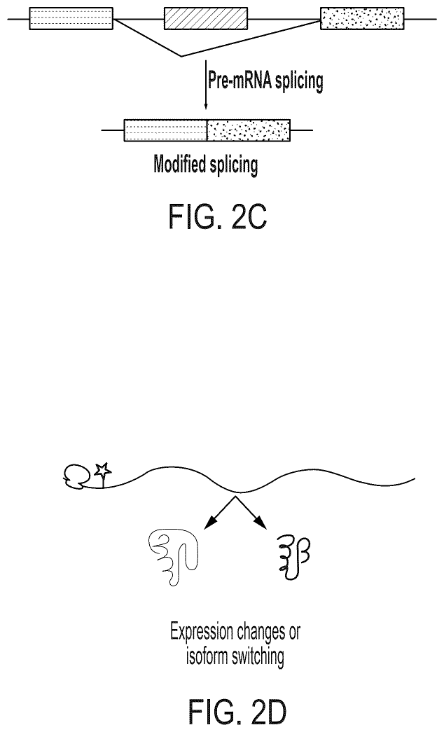

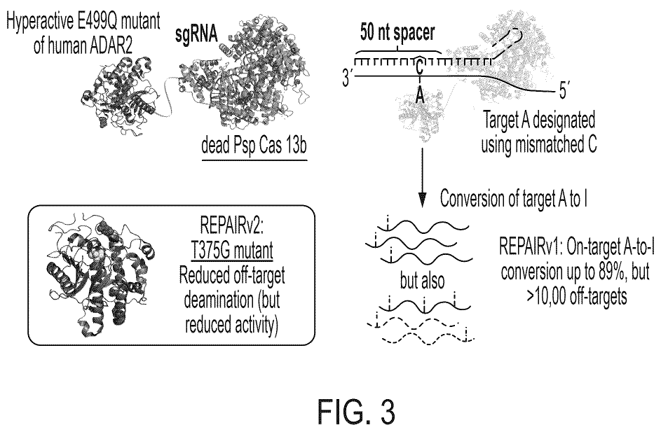

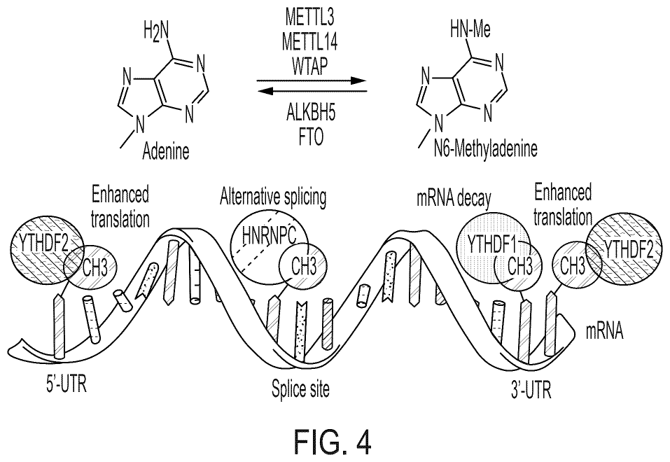

Typically, gRNAs that exist as single RNA species comprise two domains: (1) a domain that shares homology to a target nucleic acid (e.g., and directs binding of a Cas13 (or equivalent) complex to the RNA target); and (2) a domain that binds a Cas13 protein (or equivalent). In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821 (2012), the entire contents of which is incorporated herein by reference. Other examples of gRNAs (e.g., those including domain 2) can be found in U.S. Pat. No. 9,340,800, issued May 17, 2016, U.S. Pat. No. 9,228,207, issued Jan. 5, 2016, and U.S. Pat. No. 9,526,784, issued Dec. 27, 2016, the entire contents of each of which are herein incorporated by reference in their entireties. In some embodiments, a gRNA comprises two or more of domains (1) and (2), and may be referred to as an “extended gRNA.” For example, an extended gRNA will, e.g., bind two or more Cas13 proteins and bind a target RNA at two or more distinct regions, as described herein. The gRNA comprises a nucleotide sequence that complements a target site, which mediates binding of the nuclease/RNA complex to said target site, providing the sequence specificity of the nuclease: RNA complex.

Methods of using RNA-programmable nucleases, such as Cas9, for site-specific cleavage are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference). Adapting such DNA-binding Cas proteins to target RNA instead of DNA has been reported in Rauch and Dickenson, “Programmable RNA Binding Proteins for Imaging and Therapeutics,” Biochemistry, 2018, 57, pp. 363-364 (which is incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a specific site or nucleotide position in the sequence of an RNA molecule that is to become methylated or demethylated using the fusion protein disclosed herein.

The term “target RNA” refers to the specific mRNA transcript or other RNA molecule to which a RNA-programmable RNA binding domain is targeted for catalyzing the addition or removal of one or more methyl groups. The target RNA may include a gene involved in a particular disease process. For example, the target RNA may be an under-expressed gene whose low expression level is associate with a certain disease. Modulation with a writer or an editor described herein may impact the translational activity of the transcript, there by altering the level of the encoded product in a manner that may be therapeutically effective.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the term “variant” refers to a protein having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant Cas13 is a dCas13 comprising one or more changes in amino acid residues as compared to a wild type Cas13 amino acid sequence. These changes include chemical modifications, including substitutions of different amino acid residues, truncations, covalent additions (e.g. of a tag), and any other mutations. For instance, a variant Cas13 may comprise one or more amino acid substitutions that are responsible for the elimination of endoRNase activity, thus forming a catalytically inactive (or dead) Cas13. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. Cas9 protein, fusion protein, and fusion protein protein). Further polypeptides encompassed by the disclosure are polypeptides encoded by polynucleotides which hybridize to the complement of a nucleic acid molecule encoding a protein such as a Cas9 protein under stringent hybridization conditions (e.g. hybridization to filter bound DNA in 6× Sodium chloride/Sodium citrate (SSC) at about 45 degrees Celsius, followed by one or more washes in 0.2.times.SSC, 0.1% SDS at about 50-65 degrees Celsius), under highly stringent conditions (e.g. hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current Protocol in Molecular Biology, Green publishing associates, Inc., and John Wiley & Sons Inc., New York, at pp. 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Cas9 protein, can be determined conventionally using known computer programs. A preferred method for determining the best overall match between a query sequence (a sequence of the present disclosure) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

As used herein the term “wild type” is a term of the art understood by skilled persons and means the typical form of an organism, strain, gene, or characteristic as it occurs in nature as distinguished from mutant or variant forms.

### DETAILED DESCRIPTION OF CERTAIN EMBODIMENTS

The present disclosure provides programmable RNA methylation “writers” and RNA demethylation “erasers” for editing the methylation state of RNA targets. In particular, the disclosure provides RNA methylation editor constructs comprising (i) an RNA programmable RNA binding domain (RNApRNAbd); and (ii) an effector domain, wherein the effector domain is capable of adding or removing a methyl group in an RNA.

In addition, the present disclosure provides for nucleic acid molecules encoding and/or expressing the RNA methylation editors as described herein, as well as expression vectors for expressing the RNA methylation editors described herein, host cells comprising said nucleic acid molecules and expression vectors, and compositions for delivering and/or administering nucleic acid-based embodiments described herein. In addition, the disclosure provides for isolated RNA methylation editors, as well as compositions comprising said isolated RNA methylation editors as described herein.

Still further, the present disclosure provides for methods of making the RNA methylation editors, as well as methods of using the RNA methylation editors or nucleic acid molecules encoding the RNA methylation editors in applications including editing, modifying, or otherwise altering the methylation state of a target RNA molecule. The present disclosure also provides methods for efficiently editing the methylation state of a target RNA molecule with a RNA methylation editor described herein (e.g., in the form of an isolated RNA methylation editor as described herein or a vector encoding same) and conducting methylation state editing of target RNA.

In particular embodiments, the target RNA is a target sequence in a transcriptome, e.g. a mammalian transcriptome. In certain embodiments, the target RNA is a target sequence in a human transcriptome. In particular embodiments, the RNA target may be a beta-actin (ACTB) mRNA, adenosine at locus 1216 (A1216), A Disintegrin And Metalloproteinase 19 (ADAM19) mRNA or a glyceraldehyde 3-phosphate dehydrogenase (GAPDH), adenosine at locus 673 (A673).

The present disclosure provides for fusion proteins and methods of editing by use thereof that install an adenosine modification at a target sequence with specificity and accuracy. In some embodiments, the editing methods and fusion proteins disclosed herein achieve low off-target modification frequencies in mRNA sequence substrates. Accordingly, the methods and fusion proteins disclosed herein provide for high RNA editing efficiency.

In various embodiments, the disclosed fusion proteins install modifications in target RNA molecules in the cytoplasm of the target cell, the nucleus of the target cell, or both. In various embodiments, the disclosed fusion proteins install modifications with high RNA editing efficiencies (i.e., low off-target modification frequencies) in target RNA molecules in the cytoplasm of the target cell, the nucleus of the target cell, or both.

In various embodiments, the RNA programmable RNA binding domain of the RNA methylation editors of the disclosure can be a Type VI CRISPR-Cas protein, such as, a Cas13, Cas13a, Cas13b, Cas13c, or Cas13d protein or fragment thereof. In various embodiments, the RNA programmable RNA binding domain is a nuclease inactive variant of a RNA programmable RNA binding domain, e.g., a nuclease inactive (or catalytically dead) variant of Cas13, referred to a “dCas13.” In certain embodiments, the RNA programmable RNA binding domain comprises SEQ ID NO: 1 or 2. In other embodiments, the RNA programmable RNA binding domain comprises an amino acid sequence that is at least 80%, 85%, 90%, 95%, 98%, or 99% identical to the amino acid sequence of any one of SEQ ID NOs: 1 and 2.

In various other aspects, the RNA methylation editor fusion proteins described herein can comprise the structure NH₂-[RNAPRNAbd]-[effector domain]-COOH, or NH₂-[effector domain]-[RNApNAbp]-COOH, wherein each instance of “]-[” comprises an optional linker. In various embodiments, the linker that can optionally join an effector domain and an RNApNAbp can be GGGGS (SEQ ID NO: 13), GGS, SGGS (SEQ ID NO: 15), SGGSSGGS (SEQ ID NO: 22), SGSETPGTSESATPES (SEQ ID NO: 16), or SGGSSGGSSGSETPGTSESATPESSGGSSGGS (SEQ ID NO: 23).

In another aspect, the disclosure provides a fusion protein that includes (i) an amino acid sequence that is at least 80%, 85%, 90%, 95%, 98%, or 99% identical to the amino acid sequence of any one of SEQ ID NO: 1 (WP_044065294.1 (Cas13b)), or SEQ ID NO: 2 (WP_075424065.1 (Cas13d)); and (ii) an amino acid sequence that is at least 80%, 85%, 90%, 95%, 98%, or 99% identical to the amino acid sequence of any one of SEQ ID NO: 3 (Q86U44 (METTL3)), SEQ ID NO: 4 (Q9HCE5 (METTL14)), SEQ ID NO: 5 (EGR75201 (M.EcoGII)), SEQ ID NO: 6 (P9WFZ0 (TrmI)), SEQ ID NO: 7 (Q9BVS5 (Trmt61B)), SEQ ID NO: 8 (Q08J23 (Trm4)), SEQ ID NO: 9 (O14717 (Dnmt2)), or SEQ ID NO: 10 (P75876 (RlmI)), or a variant thereof.

The fusion protein for modifying RNA methylation states can also comprise (i) the amino acid sequence of any one of SEQ ID NO: 3 (Q86U44 (METTL3)), SEQ ID NO: 4 (Q9HCE5 (METTL14)), SEQ ID NO: 5 (EGR75201 (M.EcoGII)), SEQ ID NO: 6 (P9WFZ0 (TrmI)), SEQ ID NO: 7 (Q9BVS5 (Trmt61B)), SEQ ID NO: 8 (Q08J23 (Trm4)), SEQ ID NO: 9 (O14717 (Dnmt2)), or SEQ ID NO: 10 (P75876 (RlmI)), or a variant thereof; and (ii) the amino acid sequence of any one of SEQ ID NOs: 1 (Cas13b) or 2 (Cas13d).

In some embodiments, the disclosed fusion proteins comprise an effector domain comprising a methylation-inactive variants of a methyltransferase enzyme. In particular embodiments, the effector domain comprises a methylation-inactive variant of METTL3, METTL14 or METTL/METTL14 heterodimer.

In other aspects, the disclosure provides a complex comprising the fusion protein described herein complexed with a guide RNA (gRNA) bound to the RNApRNAbd of the fusion protein. The guide RNA can be a single guide RNA (sgRNA) and can be from 15-150 nucleotides long. In some embodiments, the guide RNA can have a sequence of at least 10 contiguous nucleotides that is complementary to a target sequence of an RNA. In still other embodiments, the guide RNA can have a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides that is complementary to a target sequence of an RNA.

In some embodiments of the disclosed fusion proteins, the activity of the fusion protein results in an off-target modification frequency of less than 5%, less than 3%, less than 2%, less than 1%, less than 0.75%, less than 0.7%, less than 0.65%, less than 0.6%, less than 0.55%, or less than 0.5% in target mRNA molecules. In certain embodiments, the activity of the fusion protein results in an off-target modification frequency of less than 0.7%.

In some embodiments of the disclosed fusion proteins, the activity of the fusion protein results in an off-target modification frequency of less than 5%, less than 3%, less than 2%, less than 1%, less than 0.75%, less than 0.7%, less than 0.65%, less than 0.6%, less than 0.55%, or less than 0.5% in in target RNA molecules in the cytoplasm of the target cell, the nucleus of the target cell, or both.

In some aspects, the activity of the fusion protein results in an off-target N⁶-methyladenosine (m⁶A) modification frequency of less than 5%, less than 3%, less than 2%, less than 1%, less than 0.75%, less than 0.7%, less than 0.65%, less than 0.6%, less than 0.55%, or less than 0.5% in target mRNA molecules. In other embodiments, the activity of the fusion protein results in an off-target 1-methyladenosine (m¹A) modification frequency of less than 5%, less than 3%, less than 2%, less than 1%, less than 0.75%, less than 0.7%, less than 0.65%, less than 0.6%, less than 0.55%, or less than 0.5% in the mRNA sequence substrates. In other embodiments, the activity of the fusion protein results in an off-target 5-hydroxymethylcytidine (m⁵C) modification frequency of less than 5%, less than 3%, less than 2%, less than 1%, less than 0.75%, less than 0.7%, less than 0.65%, less than 0.6%, less than 0.55%, or less than 0.5% in target mRNA molecules.

In other aspects, the present disclosure provides therapeutic methods for treating a genetic disease and/or for altering or changing a genetic trait or condition associated with an epigenetic state (e.g., methylation state) by contacting a target RNA molecule (or substrate) with an RNA methylation editor (e.g., in the form of an isolated RNA methylation editor or a vector encoding same) and conducting methylation editing to treat the genetic disease or phenotype associated with the epigenetic condition.

In some embodiments, the step of contacting a target RNA molecule results in an off-target modification frequency of less than 5%, less than 3%, less than 2%, less than 1%, less than 0.75%, less than 0.7%, less than 0.65%, less than 0.6%, less than 0.55%, or less than 0.5% in the mRNA sequence substrates. In certain embodiments, the step of contacting results in an off-target modification frequency of less than 0.7%.

In other aspects, the disclosure provides for fusion proteins and methods of editing by use thereof that require a single guide with no sequence context, wherein the fusion protein retains its ability to process its CRISPR array. Such fusion proteins are suitable for multiplexing, or the targeting of dozens, hundreds, thousands, or more sites within an RNA molecule in a single experiment. Accordingly, provided herein are methods of multiplexing using the fusion proteins described herein. In particular embodiments, provided are methods of editing more than ten, more than a hundred, more than two hundred, more than 500, more than 750, more than 1,000 or more than 10,000 loci within an RNA molecule using one or more of the disclosed fusion proteins.

### RNA Methylation Editors

In various aspects, the disclosure provides recombinant protein constructs comprising: (i) an RNA programmable RNA binding domain (RNApRNAbd); and (ii) an effector domain, wherein the effector domain is capable of adding or removing a methyl group in an RNA.

In various other aspects, the disclosure provides recombinant RNA methylation editors comprising: (i) an RNA programmable RNA binding domain (RNApRNAbd); and (ii) an effector domain, wherein the effector domain is capable of adding or removing a methyl group in an RNA.

In various embodiments, the polynucleotide constructs encoding the disclosed RNA methylation editors may include one or more linker moieties that join the RNA programmable RNA binding domains and the effector domains.

### (i) RNA Programmable RNA Binding Domain

The present disclosure embraces the use of any suitable naturally-occurring or engineered RNA programmable RNA binding domain as a fusion partner with an effector domain, such as a methyltransferase or demethylase, to achieve the desired epigenetic editing of the methylation state of an RNA target.

In certain embodiments, the RNA programmable RNA binding domain is a CRISPR/Cas protein, or homolog thereof, and in particular, a CRISPR/Cas protein having an RNA-binding activity. The CRISPR/Cas protein embraces any naturally occurring Cas from any organism, any naturally-occurring Cas equivalent or functional fragment thereof, any Cas homolog, ortholog, or paralog from any organism, and any mutant or variant of a Cas, naturally-occurring or engineered. In certain embodiments, the Cas protein used has an RN-binding activity and lacks an RNA-nuclease activity.

In one embodiment, the RNA programmable RNA binding domain is a Cas13b protein.

In an embodiment, the Cas13b protein has the amino acid sequence:

(SEQ ID NO: 1)
MNIPALVENQKKYFGTYSVMAMLNAQTVLDHIQKVADIEGEQNENNENLWF
HPVMSHLYNAKNGYDKQPEKTMFIIERLQSYFPFLKIMAENQREYSNGKYK
QNRVEVNSNDIFEVLKRAFGVLKMYRDLTNAYKTYEEKLNDGCEFLTSTEQ
PLSGMINNYYTVALRNMNERYGYKTEDLAFIQDKRFKFVKDAYGKKKSQVN
TGFFLSLQDYNGDTQKKLHLSGVGIALLICLFLDKQYINIFLSRLPIFSSY
NAQSEERRIIIRSFGINSIKLPKDRIHSEKSNKSVAMDMLNEVKRCPDELF
TTLSAEKQSRFRIISDDHNEVLMKRSSDRFVPLLLQYIDYGKLFDHIRFHV
NMGKLRYLLKADKTCIDGQTRVRVIEQPLNGFGRLEEAETMRKQENGTFGN
SGIRIRDFENMKRDDANPANYPYIVDTYTHYILENNKVEMFINDKEDSAPL
LPVIEDDRYVVKTIPSCRMSTLEIPAMAFHMFLFGSKKTEKLIVDVHNRYK
RLFQAMQKEEVTAENIASFGIAESDLPQKILDLISGNAHGKDVDAFIRLTV
DDMLTDTERRIKRFKDDRKSIRSADNKMGKRGFKQISTGKLADFLAKDIVL
FQPSVNDGENKITGLNYRIMQSAIAVYDSGDDYEAKQQFKLMFEKARLIGK
GTTEPHPFLYKVFARSIPANAVEFYERYLIERKFYLTGLSNEIKKGNRVDV
PFIRRDQNKWKTPAMKTLGRIYSEDLPVELPRQMFDNEIKSHLKSLPQMEG
IDFNNANVTYLIAEYMKRVLDDDFQTFYQWNRNYRYMDMLKGEYDRKGSLQ
HCFTSVEEREGLWKERASRTERYRKQASNKIRSNRQMRNASSEEIETILDK
RLSNSRNEYQKSEKVIRRYRVQDALLFLLAKKTLTELADFDGERFKLKEIM
PDAEKGILSEIMPMSFTFEKGGKKYTITSEGMKLKNYGDFFVLASDKRIGN
LLELVGSDIVSKED.

In another embodiment, the RNA programmable RNA binding domain is a Cas13d protein, which can have the following amino acid sequence:

(SEQ ID NO: 2)
MIEKKKSFAKGMGVKSTLVSGSKVYMTTFAEGSDARLEKIVEGDSIRSVNE
GEAFSAEMADKNAGYKIGNAKFSHPKGYAVVANNPLYTGPVQQDMLGLKET
LEKRYFGESADGNDNICIQVIHNILDIEKILAEYITNAAYAVNNISGLDKD
IIGFGKFSTVYTYDEFKDPEHHRAAFNNNDKLINAIKAQYDEFDNFLDNPR
LGYFGQAFFSKEGRNYIINYGNECYDILALLSGLAHWVVANNEEESRISRT
WLYNLDKNLDNEYISTLNYLYDRITNELTNSFSKNSAANVNYIAETLGINP
AEFAEQYFRFSIMKEQKNLGFNITKLREVMLDRKDMSEIRKNHKVFDSIRT
KVYTMMDFVIYRYYIEEDAKVAAANKSLPDNEKSLSEKDIFVINLRGSFND
DQKDALYYDEANRIWRKLENIMHNIKEFRGNKTREYKKKDAPRLPRILPAG
RDVSAFSKLMYALTMFLDGKEINDLLTTLINKFDNIQSFLKVMPLIGVNAK
FVEEYAFFKDSAKIADELRLIKSFARMGEPIADARRAMYIDAIRILGTNLS
YDELKALADTFSLDENGNKLKKGKHGMRNFIINNVISNKRFHYLIRYGDPA
HLHEIAKNEAVVKFVLGRIADIQKKQGQNGKNQIDRYYETCIGKDKGKSVS
EKVDALTKIITGMNYDQFDKKRSVIEDTGRENAEREKFKKIISLYLTVIYH
ILKNIVNINARYVIGFHCVERDAQLYKEKGYDINLKKLEEKGFSSVTKLCA
GIDETAPDKRKDVEKEMAERAKESIDSLESANPKLYANYIKYSDEKKAEEF
TRQINREKAKTALNAYLRNTKWNVIIREDLLRIDNKTCTLFANKAVALEVA
RYVHAYINDIAEVNSYFQLYHYIMQRIIMNERYEKSSGKVSEYFDAVNDEK
KYNDRLLKLLCVPFGYCIPRFKNLSIEALFDRNEAAKFDKEKKKVSGNS.

Numerous CRISPR/Cas proteins are known in the art. The present disclosure also contemplates the modification of any other these known Cas proteins to impart an RNA binding activity. Known Cas proteins may be modified by existing genetic engineering techniques to impart an RNA binding property. Known Cas proteins can be found in the following references, each of which are incorporated by reference in their entireties: (a) PCT/US2014/070038 (published as WO2015/089406, on Jun. 18, 2015) and its equivalents in the US or around the world; (b) PCT/US2016/058344 (published as WO2017/070632, on Apr. 27, 2017) and its equivalents in the US or around the world; (c) PCT/US2016/058345 (published as WO2017/070633, on Apr. 27, 2017) and its equivalent in the US or around the world; (d) PCT/US2017/045381 (published as WO2018/027078, on Feb. 8, 2018) and its equivalents in the US or around the world; (e) PCT/US2017/056671 (published as WO2018/071868, on Apr. 19, 2018) and its equivalents in the US or around the world; PCT/2017/048390 (WO2017/048390, on Mar. 23, 2017) and its equivalents in the US or around the world; (f) PCT/US2017/068114 (not published) and its equivalents in the US or around the world; (g) PCT/US2017/068105 (not published) and its equivalents in the US or around the world; (h) PCT/US2017/046144 (WO2018/031683, Feb. 15, 2018) and its equivalents in the US or around the world; (i) PCT/US2018/024208 (not published) and its equivalents in the US or around the world; (j) PCT/2018/021878 (WO2018/021878, on Feb. 1, 2018) and its equivalents in the US and around the world; (k) Komor, A. C., Kim, Y. B., Packer, M. S., Zuris, J. A. & Liu, D. R. Programmable editing of a target base in genomic DNA without double-stranded DNA cleavage. Nature 533, 420-(2016); (l) Gaudelli, N. M. et al. Programmable base editing of A-T to G-C in genomic DNA without DNA cleavage. Nature 551, 464-(2017); (m) any of the references listed in this disclosure entitled “References” and which reports or describes a base editor known in the art.

In preferred embodiments, the RNA programmable RNA binding domains is a Cas protein which lacks a nuclease activity, e.g, as described in Cox et al., 2017. In still other embodiments, the RNA programmable RNA binding domain comprises an amino acid sequence that is at least 80%, 85%, 90%, 95%, 98%, or 99% identical to the amino acid sequence of any one of SEQ ID NOs disclosed herein.

### (ii) Effector Moiety

In various embodiments, the RNA methylation editors further comprise one or more effector moieties for carrying out the RNA epigenetic editing function. In some embodiments, the effector moiety is a methyltransferase “writing” domain for adding a methyl group to an RNA molecule at a target site. In other embodiments, the effector moiety is a demethylase “eraser” domain for removing a methyl group from an RNA molecule at a target site. In still other embodiments, the effector domain comprises an amino acid sequence that is at least 80%, 85%, 90%, 95%, 98%, or 99% identical to the amino acid sequence of any one of exemplary and non-limiting examples disclosed herein, of which include:

### (A) Methyltransferases

In various embodiments, the disclosure embraces RNA methylation editor fusion proteins comprising an RNA programmable RNA binding domain fused to a methyltransferase domain, i.e., a “writer” domain. Numerous methyltransferases are known in the art and the disclosure is not particularly limited with regard to which methyltransferase may be employed. Choice of which methyltransferase can be used can depend upon various factors that include the RNA sequence context surrounding the target site, secondary RNA structure in the area of the target site, and the specific site to be modified. Without limitation, the methyltransferases can include METTL3, METTL14, M.EcoGII, TrmI, Trmt61B, Trm4, Dnmt2, and RlmI, and embraces any homolog, or variant thereof, and which may be obtained from any species or organism.

The METTL3 methyltransferase installs a methyl group in an adenine base in the sequence GGACU to form N6-methyladenosine (m⁶A) in mRNA molecules of humans (and other eukaryotes). METTL3 colocalizes with METTL14 and WTAP to form a trimeric complex. METTL14 is homologous to METTL3, but a comparison of the crystal structures within the heterodimer suggests that METTL14 is inactive.

The M.EcoGII methyltransferase installs a methyl group nonspecifically (i.e., in any sequence context) in adenine bases in DNA of Escherichia coli, as well as DNA:RNA-hybrid oligonucleotide duplexes and rA bases in RNA prepared by in vitro transcription.

The TrmI methylase installs two methyl groups at guanine26 and guanine27 to form N(2)-dimethylguanine in tRNA molecules of Thermus thermophilus. The Trmt61B methylase installs a methyl group at adenine58 to form N(1)-methyladenine in tRNA of human mitochondria. The Trm4 methylase installs a methyl group at cytosine34 to form 5-methylcytosine (m⁵C) in a CAA anticodon (corresponding to the Leu residue) in tRNA of Saccharomyces cerevisiae.

The Dnmt2 methylase installs a methyl group at cytosine38 to form 5-methylcytosine (m⁵C) in tRNA (in an anticodon corresponding to the Asp reside).

The RImI methylase installs a methyl group at cytosine1962 to form 5-methylcytosine (m⁵C) in 23S ribosomal RNA in bacteria.

In one embodiment, effector moiety is METTL3, or a functional fragment, homolog, or variant thereof. METTL3, in one embodiment, has the following amino acid sequence:

(SEQ ID NO: 3)
MSDTWSSIQAHKKQLDSLRERLQRRRKQDSGHLDLRNPEAALSPTFRSDSP
VPTAPTSGGPKPSTASAVPELATDPELEKKLLHHLSDLALTLPTDAVSICL
AISTPDAPATQDGVESLLQKFAAQELIEVKRGLLQDDAHPTLVTYADHSKL
SAMMGAVAEKKGPGEVAGTVTGQKRRAEQDSTTVAAFASSLVSGLNSSASE
PAKEPAKKSRKHAASDVDLEIESLLNQQSTKEQQSKKVSQEILELLNTTTA
KEQSIVEKFRSRGRAQVQEFCDYGTKEECMKASDADRPCRKLHFRRIINKH
TDESLGDCSFLNTCFHMDTCKYVHYEIDACMDSEAPGSKDHTPSQELALTQ
SVGGDSSADRLFPPQWICCDIRYLDVSILGKFAVVMADPPWDIHMELPYGT
LTDDEMRRLNIPVLQDDGFLFLWVTGRAMELGRECLNLWGYERVDEIIWVK
TNQLQRIIRTGRTGHWLNHGKEHCLVGVKGNPQGFNQGLDCDVIVAEVRST
SHKPDEIYGMIERLSPGTRKIELFGRPHNVQPNWITLGNQLDGIHLLDPDV
VARFKQRYPDGIISKPKNL.

In one embodiment, effector moiety is METTL14 or a functional fragment, homolog, or variant thereof. METTL14, in one embodiment, has the following amino acid sequence:

(SEQ ID NO: 4)
MDSRLQEIRERQKLRRQLLAQQLGAESADSIGAVLNSKDEQREIAETRETC
RASYDTSAPNAKRKYLDEGETDEDKMEEYKDELEMQQDEENLPYEEEIYKD
SSTFLKGTQSLNPHNDYCQHFVDTGHRPQNFIRDVGLADRFEEYPKLRELI
RLKDELIAKSNTPPMYLQADIEAFDIRELTPKFDVILLEPPLEEYYRETGI
TANEKCWTWDDIMKLEIDEIAAPRSFIFLWCGSGEGLDLGRVCLRKWGYRR
CEDICWIKTNKNNPGKTKTLDPKAVFQRTKEHCLMGIKGTVKRSTDGDFIH
ANVDIDLIITEEPEIGNIEKPVEIFHIIEHFCLGRRRLHLFGRDSTIRPGW
LTVGPTLTNSNYNAETYASYFSAPNSYLTGCTEEIERLRPKSPPPKSKSDR
GGGAPRGGGRGGTSAGRGRERNRSNFRGERGGFRGGRGGAHRGGFPPR.

In one embodiment, effector moiety is M.EcoGII, or a functional fragment, homolog, or variant thereof. M.EcoGII in one embodiment has the following amino acid sequence:

(SEQ ID NO: 5)
MLNTVKISSCELINADCLEFIRSLPENSVDLIVTDPPYFKVKPEGWDNQWK
GDDDYLKWLDQCLAQFWRVLKPAGSLYLFCGHRLASDIEIMMRERFSVLNH
IIWAKPSGRWNGCNKESLRAYFPATERILFAEHYQGPYRPKDAGYEAKGRA
LKQHVMAPLIAYFRDARAALGITAKQIADATGKKNMVPHWFSASQWQLPNE
SDYLKLQSLFARVAEEKHQRGELEKPHHQLVSTYSELNRKYMELLSEYKNL
RRYFGVTVQVPYTDVWTYKPVQYYPGKHPCEKPAEMLQQIISASSRPGDLV
ADFFMGSGSTVKAAMALGRRAIGVELETGRFEQTVREVQDLIV.

In another embodiment, effector moiety is TrmI, or a functional fragment, homolog, or

variant thereof. TrmI in one embodiment has the following amino acid sequence:

(SEQ ID NO: 6)
MSATGPFSIGERVQLTDAKGRRYTMSLTPGAEFHTHRGSIAHDAVIGLEQG
SVVKSSNGALFLVLRPLLVDYVMSMPRGPQVIYPKDAAQIVHEGDIFPGAR
VLEAGAGSGALTLSLLRAVGPAGQVISYEQRADHAEHARRNVSGCYGQPPD
NWRLVVSDLADSELPDGSVDRAVLDMLAPWEVLDAVSRLLVAGGVLMVYVA
TVTQLSRIVEALRAKQCWTEPRAWETLQRGWNVVGLAVRPQHSMRGHTAFL
VATRRLAPGAVAPAPLGRKREGRDG.

In yet another embodiment, effector moiety is Trmt61B, or a functional fragment, homolog, or variant thereof. Trmt61B in one embodiment has the following amino acid sequence:

(SEQ ID NO: 7)
MLMAWCRGPVLLCLRQGLGTNSFLHGLGQEPFEGARSLCCRSSPRDLRDGE
REHEAAQRKAPGAESCPSLPLSISDIGTGCLSSLENLRLPTLREESSPREL
EDSSGDQGRCGPTHQGSEDPSMLSQAQSATEVEERHVSPSCSTSRERPFQA
GELILAETGEGETKFKKLFRLNNFGLLNSNWGAVPFGKIVGKFPGQILRSS
FGKQYMLRRPALEDYVVLMKRGTAITFPKDINMILSMMDINPGDTVLEAGS
GSGGMSLFLSKAVGSQGRVISFEVRKDHHDLAKKNYKHWRDSWKLSHVEEW
PDNVDFIHKDISGATEDIKSLTFDAVALDMLNPHVTLPVFYPHLKHGGVCA
VYVVNITQVIELLDGIRTCELALSCEKISEVIVRDWLVCLAKQKNGILAQK
VESKINTDVQLDSQEKIGVKGELFQEDDHEESHSDFPYGSFPYVARPVHWQ
PGHTAFLVKLRKVKPQLN.

In an embodiment, the effector moiety is Trm4, or a functional fragment, homolog, or variant thereof. Trm4, in one embodiment, has the following amino acid sequence:

(SEQ ID NO: 8)
MGRRSRGRRLQQQQRPEDAEDGAEGGGKRGEAGWEGGYPEIVKENKLFEHY
YQELKIVPEGEWGQFMDALREPLPATLRITGYKSHAKEILHCLKNKYFKEL
EDLEVDGQKVEVPQPLSWYPEELAWHTNLSRKILRKSPHLEKFHQFLVSET
ESGNISRQEAVSMIPPLLLNVRPHHKILDMCAAPGSKTTQLIEMLHADMNV
PFPEGFVIANDVDNKRCYLLVHQAKRLSSPCIMVVNHDASSIPRLQIDVDG
RKEILFYDRILCDVPCSGDGTMRKNIDVWKKWTTLNSLQLHGLQLRIATRG
AEQLAEGGRMVYSTCSLNPIEDEAVIASLLEKSEGALELADVSNELPGLKW
MPGITQWKVMTKDGQWFTDWDAVPHSRHTQIRPTMFPPKDPEKLQAMHLER
CLRILPHHQNTGGFFVAVLVKKSSMPWNKRQPKLQGKSAETRESTQLSPAD
LTEGKPTDPSKLESPSFTGTGDTEIAHATEDLENNGSKKDGVCGPPPSKKM
KLFGFKEDPFVFIPEDDPLFPPIEKFYALDPSFPRMNLLTRTTEGKKRQLY
MVSKELRNVLLNNSEKMKVINTGIKVWCRNNSGEEFDCAFRLAQEGIYTLY
PFINSRIITVSMEDVKILLTQENPFFRKLSSETYSQAKDLAKGSIVLKYEP
DSANPDALQCPIVLCGWRGKASIRTFVPKNERLHYLRMMGLEVLGEKKKEG
VILTNESAASTGQPDNDVTEGQRAGEPNSPDAEEANSPDVTAGCDPAGVHP
PR.

In an embodiment, the effector moiety is Dnmt2, or a functional fragment, homolog, or variant thereof. Dnmt2, in one embodiment, has the following amino acid sequence:

(SEQ ID NO: 9)
MEPLRVLELYSGVGGMHHALRESCIPAQVVAAIDVNTVANEVYKYNFPHTQ
LLAKTIEGITLEEFDRLSFDMILMSPPCQPFTRIGRQGDMTDSRTNSFLHI
LDILPRLQKLPKYILLENVKGFEVSSTRDLLIQTIENCGFQYQEFLLSPTS
LGIPNSRLRYFLIAKLQSEPLPFQAPGQVLMEFPKIESVHPQKYAMDVENK
IQEKNVEPNISFDGSIQCSGKDAILFKLETAEEIHRKNQQDSDLSVKMLKD
FLEDDTDVNQYLLPPKSLLRYALLLDIVQPTCRRSVCFTKGYGSYIEGTGS
VLQTAEDVQVENIYKSLTNLSQEEQITKLLILKLRYFTPKEIANLLGFPPE
FGFPEKITVKQRYRLLGNSLNVHVVAKLIKILYE.

In an embodiment, the effector moiety is RlmI, or a functional fragment, homolog, or variant thereof. RlmI, in one embodiment, has the following amino acid sequence:

(SEQ ID NO: 10)
MSVRLVLAKGREKSLLRRHPWVFSGAVARMEGKASLGETIDIVDHQGKWLA
RGAYSPASQIRARVWTFDPSESIDIAFFSRRLQQAQKWRDWLAQKDGLDSY
RLIAGESDGLPGITIDRFGNFLVLQLLSAGAEYQRAALISALQTLYPECSI
YDRSDVAVRKKEGMELTQGPVTGELPPALLPIEEHGMKLLVDIQHGHKTGY
YLDQRDSRLATRRYVENKRVLNCFSYTGGFAVSALMGGCSQVVSVDTSQEA
LDIARQNVELNKLDLSKAEFVRDDVFKLLRTYRDRGEKFDVIVMDPPKFVE
NKSQLMGACRGYKDINMLAIQLLNEGGILLTFSCSGLMTSDLFQKIIADAA
IDAGRDVQFIEQFRQAADHPVIATYPEGLYLKGFACRVM.

### (B) Demethylases

In various embodiments, the disclosure embraces RNA methylation editor fusion proteins comprising an RNA programmable RNA binding domain fused to a demethylase domain, i.e., an “eraser” domain. Numerous demethylases are known in the art and the disclosure is not particularly limited with regard to which demethylase may be employed. Choice of which demethylase can be used can depend upon various factors that include the RNA sequence context surrounding the target site, secondary RNA structure in the area of the target site, and the specific site to be modified. Without limitation, the demethylases can include ALKBH5 and FTO, and embraces any homolog, or variant thereof, and which may be obtained from any species or organism.

In an embodiment, the effector moiety is ALKBH5 or a functional fragment, homolog, or variant thereof. ALKBH5, in one embodiment, has the following amino acid sequence:

(SEQ ID NO: 11)
MAAASGYTDLREKLKSMTSRDNYKAGSREAAAAAAAAVAAAAAAAAAAEPY
PVSGAKRKYQEDSDPERSDYEEQQLQKEEEARKVKSGIRQMRLFSQDECAK
IEARIDEVVSRAEKGLYNEHTVDRAPLRNKYFFGEGYTYGAQLQKRGPGQE
RLYPPGDVDEIPEWVHQLVIQKLVEHRVIPEGFVNSAVINDYQPGGCIVSH
VDPIHIFERPIVSVSFFSDSALCFGCKFQFKPIRVSEPVLSLPVRRGSVTV
LSGYAADEITHCIRPQDIKERRAVIILRKTRLDAPRLETKSLSSSVLPPSY
ASDRLSGNNRDPALKPKRSHRKADPDAAHRPRILEMDKEENRRSVLLPTHR
RRGSFSSENYWRKSYESSEDCSEAAGSPARKVKMRRH.

In another embodiment, the effector moiety is FTO, or a functional fragment, homolog, or variant thereof. FTO, in one embodiment, has the following amino acid sequence:

(SEQ ID NO: 12)
MKRTPTAEEREREAKKLRLLEELEDTWLPYLTPKDDEFYQQWQLKYPKLIL
REASSVSEELHKEVQEAFLTLHKHGCLFRDLVRIQGKDLLTPVSRILIGNP
GCTYKYLNTRLFTVPWPVKGSNIKHTEAEIAAACETFLKLNDYLQIETIQA
LEELAAKEKANEDAVPLCMSADFPRVGMGSSYNGQDEVDIKSRAAYNVTLL
NFMDPQKMPYLKEEPYFGMGKMAVSWHHDENLVDRSAVAVYSYSCEGPEEE
SEDDSHLEGRDPDIWHVGFKISWDIETPGLAIPLHQGDCYFMLDDLNATHQ
HCVLAGSQPRFSSTHRVAECSTGTLDYILQRCQLALQNVCDDVDNDDVSLK
SFEPAVLKQGEEIHNEVEFEWLRQFWFQGNRYRKCTDWWCQPMAQLEALWK
KMEGVTNAVLHEVKREGLPVEQRNEILTAILASLTARQNLRREWHARCQSR
IARTLPADQKPECRPYWEKDDASMPLPFDLTDIVSELRGQLLEAKP.

In certain embodiments, the disclosed fusion proteins comprise a dCas13-M3nes, a dCas13-M3nls, a dCas13-M3M14nes, or a dCas13-M3M14nls. In particular embodiments, the disclosed fusion proteins comprise a Cas13-M3nls.

In certain embodiments of the polynucleotide constructs of the present disclosure, the disclosed constructs comprise a dCas13-M3nes construct, a dCas13-M3nls construct, a dCas13-M3M14nes construct, or a dCas13-M3M14nls construct. In particular embodiments, the disclosed fusion proteins comprise a Cas13-M3nls construct.

In certain embodiments, the disclosed fusion proteins comprise the amino acid sequence of any one of SEQ ID NOs: 24-27. In particular embodiments, the disclosed fusion proteins comprise the amino acid sequence of SEQ ID NO: 25.

(iii) Linkers

In certain embodiments, linkers may be used to link any of the peptides or peptide domains or moieties of the disclosure. In various embodiments, the RNA programmable RNA binding domain can be joined through a linker to one or more effector domains. In certain embodiments, the RNA programmable RNA binding domain is linked to a methyltransferase domain through a linker. In other embodiments, the RNA programmable RNA binding domain is linked to a demethylase domain through a linker. The order of the domains on either side of the linker is non-limiting; thus, the disclosure embraces fusion proteins that comprise RNA programmable RNA binding domains linked to an effector domain, as well as an effector domain linked to an RNA programmable RNA binding domain. Thus, either order is embraced herein. The fusion proteins typically, in various embodiments, are expressed as translational fusion products from a nucleotide sequence encoding the RNA programmable RNA binding domain, the linker, and the effector domain, or in other embodiments, the effector domain, the linker, and the RNA programmable RNA binding domain.

As defined above, the term “linker,” as used herein, refers to a chemical group or a molecule linking two molecules or moieties, e.g., an RNA programmable RNA binding domain and a demethylase or methyltransferase domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated

In some other embodiments, the linker comprises the amino acid sequence (GGGGS)ₙ (SEQ ID NO: 13), (G)ₙ (SEQ ID NO: 35), (EAAAK)ₙ (SEQ ID NO: 14), (GGS)ₙ (SEQ ID NO: 36), (SGGS)ₙ (SEQ ID NO: 15), SGSETPGTSESATPES (SEQ ID NO: 16), (XP)ₙ (SEQ ID NO: 37), or any combination thereof, wherein n is independently an integer between 1 and 30, and wherein X is any amino acid. In some embodiments, the linker comprises the amino acid sequence (GGS)ₙ, wherein n is 1, 3, or 7 (SEQ ID NO: 38). In some embodiments, the linker comprises the amino acid sequence SGSETPGTSESATPES (SEQ ID NO: 16). In other embodiments, the linker is SGGSSGGS (SEQ ID NO: 22) or SGGSSGGSSGSETPGTSESATPESSGGSSGGS (SEQ ID NO: 23).

### (iv) Nuclear Localization Signals

In various embodiments, the RNA methylation editors disclosed herein further comprise one or more, nuclear localization signals (NLS). In a preferred embodiment, the RNA methylation editors comprise at least two NLSs. In embodiments the NLSs can be the same NLSs or they can be different NLSs. In addition, the NLSs may be expressed as part of a fusion protein with the remaining portions of the RNA methylation editors. The location of the NLS fusion can be at the N-terminus, the C-terminus, or within a sequence of a base editor (e.g., inserted between the encoded napR/DNAbp component (e.g., Cas13) and a DNA effector moiety (e.g., a methyltransferase)).

The NLS of the disclosed fusion proteins may be any NLS sequence known in the art. The NLSs may also be any future-discovered NLSs for nuclear localization. The NLSs also may be any naturally-occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for their disclosure of exemplary nuclear localization sequences. In some embodiments, a NLS comprises the amino acid sequence PKKKRKV (SEQ ID NO: 17), MDSLLMNRRKFLYQFKNVRWAKGRRETYLC (SEQ ID NO: 18), KRTADGSEFESPKKKRKV (SEQ ID NO: 19), or KRTADGSEFEPKKKRKV (SEQ ID NO: 20).

In one aspect of the disclosure, an RNA methylation editor may be modified with one or more nuclear localization signals (NLS). In certain embodiments, the RNA methylation editors are modified with two or more NLSs. The disclosure contemplates the use of any nuclear localization signal known in the art at the time of the disclosure, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Leff. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV SEQ ID NO: 17); (ii) a bipartite motif consisting of two basic domains separated by a variable number of spacer amino acids and exemplified by the Xenopus nucleoplasmin NLS (KRXXXXXXXXKKKL SEQ ID NO: 21); and (iii) noncanonical sequences such as M9 of the hnRNP A1 protein, the influenza virus nucleoprotein NLS, and the yeast Gal4 protein NLS (Dingwall and Laskey 1991).

Nuclear localization signals appear at various points in the amino acid sequences of proteins. NLS's have been identified at the N-terminus, the C-terminus and in the central region of proteins. Thus, the disclosure provides RNA methylation editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a RNA methylation editor to include one or more NLSs. In one aspect, the RNA methylation editors can be engineered to express a base editor protein that is translationally fused at its N-terminus or its C-terminus (or both) to one or more NLSs, i.e., to form a base editor-NLS fusion polynucleotide, or polynucleotide construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence, e.g, and in the central region of proteins. Thus, the present disclosure also provides for nucleotide constructs, vectors, and host cells for expressing fusion proteins that comprise a base editor and one or more NLSs.

The improved RNA methylation editors described herein may also comprise nuclear localization signals which are linked to a base editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker moiety) and be joined to the base editor by any suitable strategy that effectuates forming a bond (e.g., covalent linkage, hydrogen bonding) between the base editor and the one or more NLSs.

Accordingly, in some embodiments, the disclosed fusion proteins have a structure of NH₂-[NLS]-[RNApRNAbd]-[effector domain]-COOH, or NH₂-[effector domain]-[RNApNAbp]-[NLS]-COOH, wherein each instance of “]-[” comprises an optional linker.

### (v) Nuclear Export Signals

In various embodiments, the RNA methylation editors disclosed herein further comprise one or more, nuclear export signals (NES). In a preferred embodiment, the RNA methylation editors comprise at least two NESs. In embodiments the NESs can be the same NESs or they can be different NESs. In addition, the NESs may be expressed as part of a fusion protein with the remaining portions of the RNA methylation editors. The location of the NES fusion can be at the N-terminus, the C-terminus, or within a sequence of a base editor (e.g., inserted between the encoded napR/DNAbp component (e.g., Cas13) and a DNA effector moiety (e.g., a methyltransferase)).

The NES of the disclosed fusion proteins may be any NES sequence known in the art. The NES may be any future-discovered NESs for nuclear export. The NES may be any naturally-occurring NES, or any non-naturally occurring NES (e.g., an NES with one or more desired mutations). In particular embodiments, the NES is an HIV viral NES.

The present disclosure contemplates any suitable means by which to modify a RNA methylation editor to include one or more NESs. In one aspect, the RNA methylation editors can be engineered to express a base editor protein that is translationally fused at its N-terminus or its C-terminus (or both) to one or more NESs, i.e., to form a base editor-NES fusion construct. In other embodiments, the base editor-encoding nucleotide sequence can be genetically modified to incorporate a reading frame that encodes one or more NESs in an internal region of the encoded base editor. In addition, the NESs may include various amino acid linkers or spacer regions encoded between the base editor and the N-terminally, C-terminally, or internally-attached NESs amino acid sequence, e.g, and in the central region of proteins. Thus, the present disclosure also provides for nucleotide constructs, vectors, and host cells for expressing fusion proteins that comprise a base editor and one or more NESs.

In some embodiments, the NES of the disclosed fusion proteins comprises an HIV nuclear export signal. In some embodiments, the NES comprises the amino acid sequence LQLPPLERLTL (SEQ ID NO: 34).

Accordingly, in some embodiments, the disclosed fusion proteins have a structure of NH₂-[NES]-[RNApRNAbd]-[effector domain]-COOH, or NH₂-[effector domain]-[RNApNAbp]-[NES]-COOH, wherein each instance of “]-[” comprises an optional linker.

### (vi) Additional Components of the Fusion Proteins

The RNA methylation editors described herein may comprise one or more heterologous protein domains (e.g., about or more than about 1, 2, 3, 4, 5, 6, 7, 8, 9, 10, or more domains in addition to the base editor components). A RNA methylation editor may comprise any additional protein sequence, and optionally a linker sequence between any two domains. Examples of protein domains that may be fused to the RNA methylation editors described herein include, without limitation, epitope tags, reporter gene sequences, and protein domains having one or more of the following activities: methyltransferase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a base editor are described in US20110059502, incorporated herein by reference. In some embodiments, a tagged base editor is used to identify the location of a target sequence.

In an aspect of the disclosure, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the disclosure, the DNA molecule encoding the gene product may be introduced into the cell via a vector. In a preferred embodiment of the disclosure the gene product is luciferase. In a further embodiment of the disclosure the expression of the gene product is decreased.

Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein comprises one or more His tags.

(vii) the Guide Sequence (e.g., a Guide RNA)

In various embodiments, the RNA methylation editors can be complexed, bound, or otherwise associated with (e.g., via any type of covalent or non-covalent bond) one or more appropriate guide sequences, i.e., the sequence which becomes associated or bound to the RNA methylation editor and which direct its localization to a specific target RNA sequence having complementarity to the guide sequence or a portion thereof. The particular design aspects of a guide sequence will depend upon the nucleotide sequence of a RNA target site of interest (i.e., the desired site to undergo methylation editing) and the type of Cas protein (e.g., Cas13) present in the RNA methylation editor, among other factors.

In general, a guide sequence is any polynucleotide sequence having sufficient complementarity with a target polynucleotide sequence to hybridize with the target sequence and direct sequence-specific binding of a RNA methylation editor to the target sequence. The appropriate design and mRNA accessibility of guide sequences that are suitable for dCas13 mRNA editing at suitable (including high) on-target editing frequencies can be predicted using the RNApl fold algorithm in the Vienna RNA software suite. See Bernhart, S. H., Hofacker, I. L. & Stadler, P. F. Local RNA base pairing probabilities in large sequences. Bioinformatics 22(5): 614-615 (2006), herein incorporated by reference. This software is publicly accessible at the URL, http://www.tbi.univie.ac.at/RNA/.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within an RNA transcriptome of a cell.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. Patent Publication No. 2016/0340622, incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the disclosure, the transcript or transcribed polynucleotide sequence has at least two or more hairpins. In preferred embodiments, the transcript has two, three, four or five hairpins. In a further embodiment of the disclosure, the transcript has at most five hairpins. In some embodiments, the single transcript further includes a transcription termination sequence; preferably this is a polyT sequence, for example six T nucleotides.

It will be apparent to those of skill in the art that in order to target any of the fusion proteins comprising a Cas domain (e.g., Cas13d) and an effector domain, as disclosed herein, to a target site, e.g., a site comprising a methylation site in an RNA to be changed, it is typically necessary to co-express the fusion protein together with a guide RNA, e.g., an sgRNA. As explained in more detail elsewhere herein, a guide RNA typically comprises a tracrRNA framework allowing for Cas binding, and a guide sequence, which confers sequence specificity to the Cas fusion protein.

In some embodiments, the guide RNA comprises a structure 5′-[guide sequence]-guuuuagagcuagaaauagcaaguuaaaauaaggcuaguccguuaucaacuugaaaaaguggcaccgagucggugcuuuuu-3′, wherein the guide sequence comprises a sequence that is complementary to the target sequence. The guide sequence is typically 20 nucleotides long. Exemplary guide sequences for efficient targeting of the mRNA targets of the Examples are disclosed in Table 2, and correspond to SEQ ID NOs: 29-33. The sequences of suitable guide RNAs for targeting the disclosed fusion proteins to specific RNA target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Exemplary guide RNA sequences suitable for targeting any of the provided fusion proteins to specific target sequences are provided herein in Table 2. Additional guide sequences are well known in the art and can be used with the RNA methylation editors described herein.

### Methods of Making RNA Methylation Editors

Several aspects of the making and using the RNA methylation editors of the disclosure relate to vector systems comprising one or more vectors, or vectors as such. Vectors can be designed to clone and/or express the RNA methylation editors of the disclosure. Vectors can also be designed to transfect the RNA methylation editors of the disclosure into one or more cells, e.g., a target diseased eukaryotic cell for treatment with the base editor systems and methods disclosed herein.

Vectors can be designed for expression of RNA methylation editor transcripts (e.g. nucleic acid transcripts, proteins, or enzymes) in prokaryotic or eukaryotic cells. For example, RNA methylation editor transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press. San Diego, Calif. (1990). Alternatively, expression vectors encoding one or more improved RNA methylation editors described herein can be transcribed and translated in vitro, for example using T7 promoter regulatory sequences and T7 polymerase.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion expression vectors also may be used to express the RNA methylation editors of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector for expressing the improved RNA methylation editors described herein. Examples of vectors for expression in yeast Saccharomyces cerevisae include p YepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39).

In some embodiments, a vector is capable of driving expression of one or more sequences in mammalian cells using a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546).

### Methods of Using RNA Methylation Editors

Some aspects of this disclosure provide methods of using the RNA methylation editors disclosed herein for introducing one or more changes in the methylation state of an RNA. On other aspects, this disclosure provide methods of using the RNA methylation editors disclosed herein for globally changing the epitranscriptome state of a cell, e.g., the methylation state of the expressed transcripts of a cells. In still other aspects, the disclosure provides methods of treating a subject having a disease or condition that is caused by a first methylation state of the transcriptome comprising contacting the diseased cells with an RNA methylation editor of the disclosure, thereby altering the methylation state of the transcriptome to a second, but non-disease associated state.

The instant disclosure provides methods for the treatment of a subject diagnosed with a disease associated with or caused by an aberrant state of RNA methylation. For example, in some embodiments, a method is provided that comprises administering to a subject having such an RNA methylation state-associated disease, e.g., a cancer associated with an aberrant methylation state, an effective amount of a RNA methylation editor described herein that removes the disease-causing methylation state. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease.

In some embodiments, the disease is a cancer. In particular embodiments, the disease is glioblastoma, acute myeloid leukemia, or breast cancer. In some embodiments, the disease is associated with antitumor immunity, learning and memory, neuronal regeneration, stem cell differentiation, and/or neurodegeneration.

### Pharmaceutical Compositions

Other aspects of the present disclosure relate to pharmaceutical compositions comprising any of the fusion proteins or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for editing the RNA transcriptome methylation state. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the disclosure in lyophilized form and (b) a second container containing a pharmaceutically acceptable diluent (e.g., sterile water) for injection. The pharmaceutically acceptable diluent can be used for reconstitution or dilution of the lyophilized compound of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

In some aspects, the disclosure provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For reviews of gene delivery procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51(1):31-44 (1995); Haddada et al., Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US Publication No. 2003/0087817, incorporated herein by reference.

Some aspects of this disclosure provide kits comprising a nucleic acid construct comprising a nucleotide sequence encoding an RNA methylation editor described herein. In some embodiments, the nucleotide sequence comprises a heterologous promoter that drives expression of the RNA methylation editors.

Some aspects of this disclosure provide kits comprising a nucleic acid construct, comprising (a) a nucleotide sequence encoding an RNA-programmable RNA binding protein (e.g., Cas13) fused to an effector domain; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some aspects of this disclosure provide cells comprising any of the RNA methyltransferases editors, RNA demethylase editors, fusion proteins, or complexes provided herein.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-m⁶A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

The description of exemplary embodiments of the reporter systems above is provided for illustration purposes only and not meant to be limiting. Additional reporter systems, e.g., variations of the exemplary systems described in detail above, are also embraced by this disclosure.

It should be appreciated however, that additional fusion proteins would be apparent to the skilled artisan based on the present disclosure and knowledge in the art.

The function and advantage of these and other embodiments of the present disclosure will be more fully understood from the Examples below. The following Examples are intended to illustrate the benefits of the present disclosure and to describe particular embodiments, but are not intended to exemplify the full scope of the disclosure. Accordingly, it will be understood that the Examples are not meant to limit the scope of the disclosure.

### EXAMPLES

### Example 1: Development of a Programmable Writer and Eraser of m⁶A RNA Methylation

m⁶A modifications at the RNA level has quickly risen as a biologically important, dynamic modification that effects the overall functional outcomes of RNA transcripts. Currently, there is no way of specifically targeting the addition or removal of methylation sites. To this end, the Example shows the construction of a programmable methylation “writer” by fusing METTL3 to the newly discovered RNA-targeting Cas13b. In addition, this Example constructs a programmable demethylation “eraser” by fusing ALKBH5 to Cas13b.

### Experimental Methods

Kinetics of METTL3, METTL3-14 and METTL14. METTL cDNAs were synthesized by Genscript with primary codon optimization mammalian and secondary optimization K12 E. coli. Synthesized genes were sub-cloned into pET-41M vector containing a His-tag and MBP-tag on the N-terminus. The resulting vector was transformed into BL21 cells and grown in autoinduction media at 37 degrees to an OD 0.6 switched to 16° C. and grown for additional 16 hours. Cells were lysed in the presence of lysozyme, benzonase and protease inhibitors by sonication. After purification via a Talon- and MBP-column, the tags were cleaved with His-tagged TEV-protease overnight at 4° C. while dialyzing against storage buffer (20 mM Tris-HCl pH 7.5, 5% (v/v) glycerol). Cleaved sample was collected and run over Ni-NTA column to remove His-tagged TEV, cleaved MBP and uncleaved His-MBP-METTLX contaminants. Flow-through was collected, concentrated to 10 mL aliquoted and stored at −80° C.

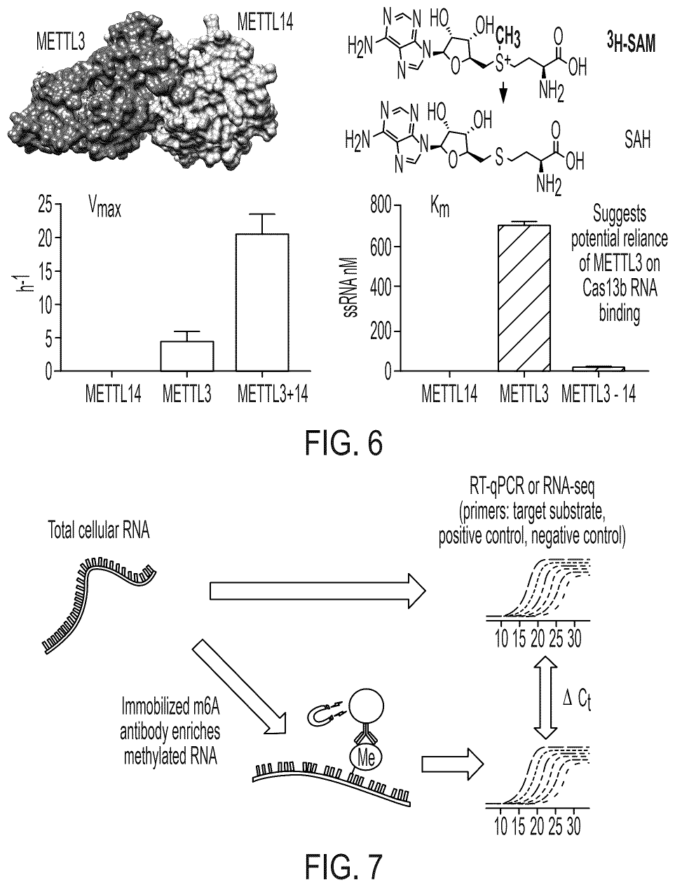

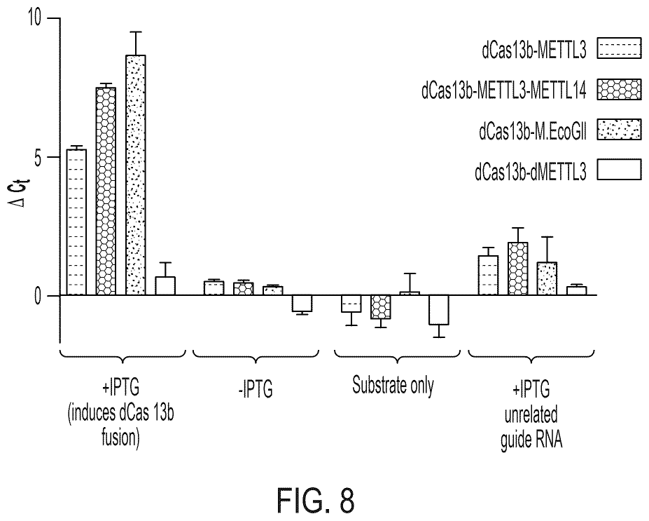

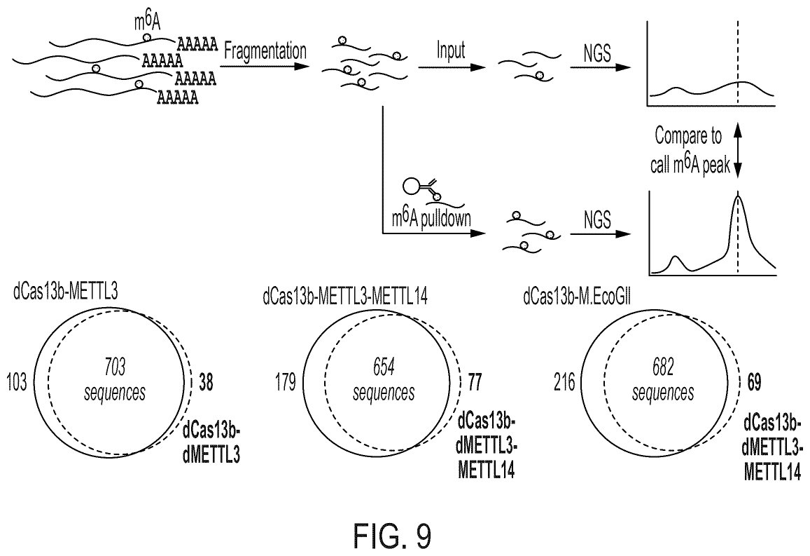

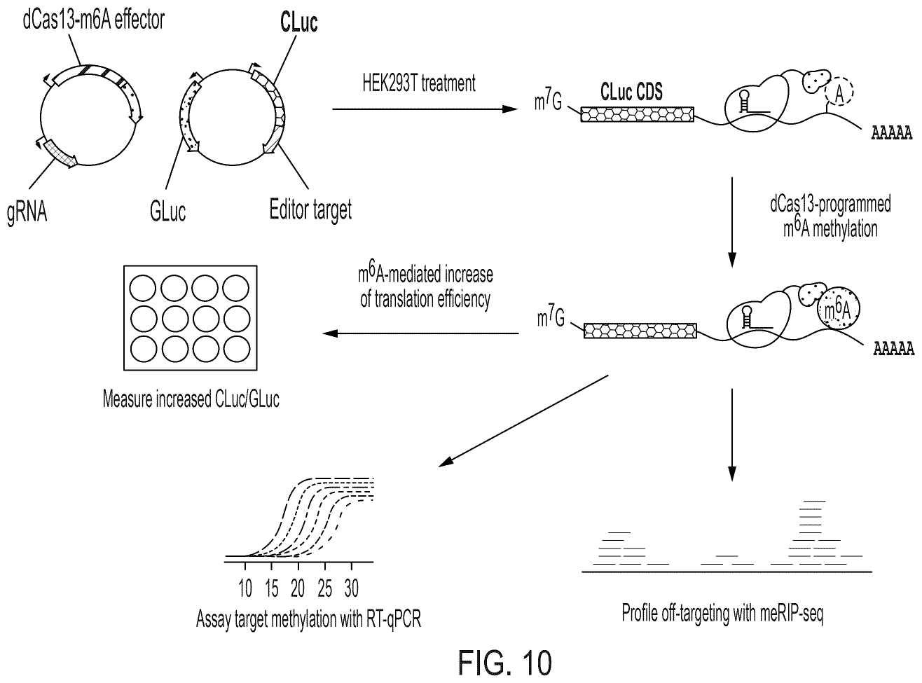

The methyltransferase activity of METTL constructs were measured with a radioactivity-based assay. In this assay, radiolabeled S-adenosyl-L-methionine (³H-SAM) and unmethylated N6-adenine single stranded RNA labeled with dual biotins on the 5′ end (IDT) were used as substrates. See FIG. 6. After reaction, the biotinylated ss-RNA was captured in a FlashPlate coated with streptavidin/scintillant. The amount of methylated m⁶A ss-RNA was quantified by scintillation counting using a Topcount reader. All assays were performed using 20 mM Tris pH 7.5, 1 mM DTT, 0.01% NP-50, 40 U of RNaseOUT. For determination of kinetic parameters, protein concentrations and reaction time were optimized to obtain linear initial velocities To determine the Km values for ss-RNA SAM concentration was locked at 20 uM and ss-RNA was varied from 0 to 2 uM. The initial velocities of the resulting curves where fit to create a Michaelis-Menten curve Km and Vmax values were calculated using the Prism software package.

Cellular RNA Methylation Assays and Expression. BL21 Tuner Cells (EMD) were transformed with two vectors. One vector constitutively expressed (J23119 vector) a target substrate containing a target sequence surrounded by canonical m⁶A sites. A second vector containing a dCas13b-METTL3, dCas13b-METTL3-METTL14, dCas13b-M.EcogII or dCas13b-dMETTL3 fusion under inducible expression (T7) and a constitutively expressed guide RNA with a spacer targeting the target substrate.

RNA purification and m⁶A immunoprecipitation. Cells where lysed and total RNA purified by Trizol/chloroform extraction (Invitrogen) followed by Rneasy column purification (Qiagen). Purified Total RNA was ribodepleted using a Ribozero bacterial gold kit (illumina) and chemically fragmented to ˜200 bp and spiked with positive and negative control m⁶A RNA's. One half was saved while the other half was Immunoprecipitated. Briefly immunoprecipitation was performed by attachment of N6-methyladenosine antibody (NEB) to Protein G magnetic beads (Invitrogen). ˜50 ug of ribodepleted RNA was added to m⁶A-antibody-magnetic beads and rotated for 2 hours at 4° C. Beads where washed 5× times with 3 buffers a high salt buffer (500 mM NaCl, 10 mM Tris-HCl, pH 7.5, 0.1% NP-40 in nuclease free H₂O) a low salt buffer ((50 mM NaCl, 10 mM Tris-HCl, pH 7.5, 0.1% NP-40 in nuclease free H₂O) and reaction buffer Reaction Buffer (150 mM NaCl, 10 mM Tris-HCl, pH 7.5, 0.1% NP-40 in nuclease free H₂O). RNA was eluted with RLT buffer (Qiagen).

meRIP-RT-qPCR. meRIP-RT-qPCR was performed by using ˜2 ng of ribodepleted RNA and IP-ribodepleted RNA as template specific primers for the positive control RNA, negative control RNA and target RNA and RNA to CT one step RT-qPCR master mix (Invitrogen) and run on a Bio-Rad Cfx96 real time qPCR machine. Ct values where calculated by bio-rad software.

Fishing ELISA Experiment. A second orthogonal experiment was performed to ensure target substrate methylation was occurring. A dual biotin probe for the target sequence was hybridized to ˜50 μg of total RNA. Hybridized RNA was applied to a streptavidin mini column (uMACS) and washed 10× times with high salt buffer (500 mM NaCl, 10 mM Tris-HCl, pH 7.5, 0.1% NP-40 in nuclease free H2O) to remove non-hybridized RNA. To elute nuclease free water heated to 85 degrees was then added and the first 4 drop collected. RNA concentration was than quantified. Eluted RNA of varying concentrations 2 ng to 50 μg was applied to a RNA binding plate (Epigentek), the wells where then washed and an m⁶A specific antibody was added (Epigentek) and second detection antibody was added (Epigentek). Fluorescence was read on a Tecan microplate reader.

meRIP-Seq. In order to assess off-targeting effects, meRIP-seq was performed by taking ribodepleted RNA and IP ribodepleted RNA and performing RNA-seq on both using the Trueseq RNA library prep kit 2 (Illumina). Prepared libraries where sequenced on an inhouse Nextseq 550 sequencer using a High throughput flow cell. Reads from the control (ribodepleted RNA) and IP where aligned to K-12 E. coli genome using the HISAT2 software package resulting BAM files where used to locate methylation sites using the Exomepeak program executed in R (free programming language supported by the R Foundation for Statistical Computing).

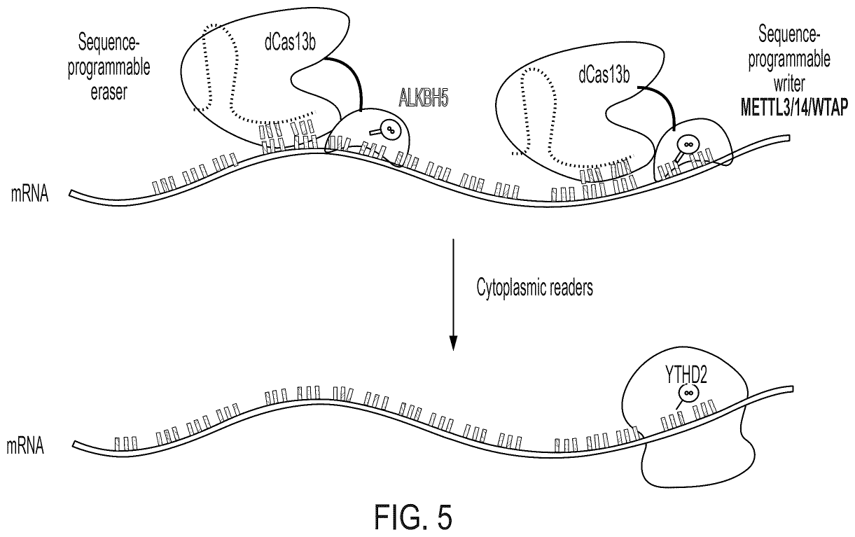

In the presently disclosed Examples, a Cas13b-METTL3 and Cas13b-ALKHB5 fusion to create a programmable m⁶A “writer” and “eraser” was created in the hopes of both furthering basic research on the specific effects of m⁶A methylation and understanding and treating human diseases, including cancer. First, Cas13b-METTL3 fusion protein with optimized efficiency and specificity of methylation of adenines along a target mRNA was constructed. Second, an m⁶A “eraser” was constructed by fusing ALKBH5 with Cas13b to specifically remove methylated adenines at targeted sites. This system was first validated in an E. coli system and subsequently validated in the biologically relevant context of human cells (see FIG. 5).

### Construction of the “Writer”

### Part 1. In Vitro Screening for Km Impaired METTL3 Construct

The crystal structure suggests that METTL3 is the active methyltransferase and that METTL14 and WTAP are accessory proteins that most likely assist METTL3 in binding ^{[18, 24]}. Unlike METTL14, METTL3 has an intact SAM methyltransferase active site, suggesting that METTL3 on its own may be capable of the methyltransferase reaction, albeit with impaired mRNA binding (Km). If METTL3 is active but biologically impaired, due to a Km greater than the concentration of its target mRNA, tethering the domain to Cas13b may overcome the impaired Km due to an increase in local concentration. Such a situation would be advantageous as it would link METTL3 activity to the binding of Cas13b to the target mRNA strand, which in turn would provide specificity.

Michaelis-Menten kinetics of the METTL3/14 complex and METTL3 alone were compared using a commercially available kit that follows SAM depletion (data not shown). In conjunction with the kit assay, a radiometric assay was used, which monitors the transfer of a C14 labelled methyl group from SAM to a biotin-labelled RNA. This transfer is followed by capture of the RNA on a streptavidin flash plate and counted using a scintillation microplate counter ^{[26]}. It was found that the Km of METTL3 was severely impaired by two orders of magnitude without the METTL14 complexed, while the Vmax was modestly reduced 2-3 fold. See FIG. 6. Thus, METTL3 may be the ideal fusion to Cas13b, as the increase in local concentration provided by the Cas13b would overcome the weak Km of METTL3.

### Part 2. In Vitro Screening for Km Impaired METTL3 Construct

The crystal structure suggests that METTL3 is the active methyltransferase and that METTL14 and WTAP are accessory proteins that most likely assist METTL3 in binding ^{[18, 24]}. Unlike METTL14, METTL3 has an intact SAM methyltransferase active site, suggesting that METTL3 on its own may be capable of the methyltransferase reaction, albeit with impaired mRNA binding (Km). If METTL3 is active but biologically impaired, due to a Km greater than the concentration of its target mRNA, tethering to Cas13b may overcome the impaired Km due to an increase in local concentration. Such a situation would be advantageous as it would link METTL3 activity to the binding of Cas13b to the target mRNA strand, which in turn would provide specificity.

### Part 3. Bacterial Screen for Cas13b-METTL3 Construct Activity

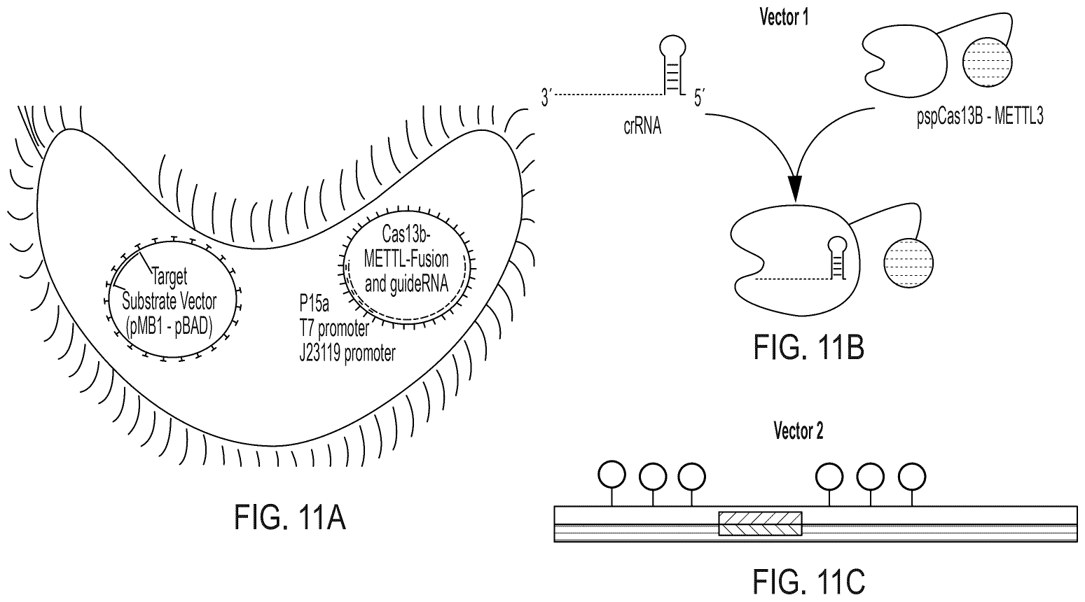

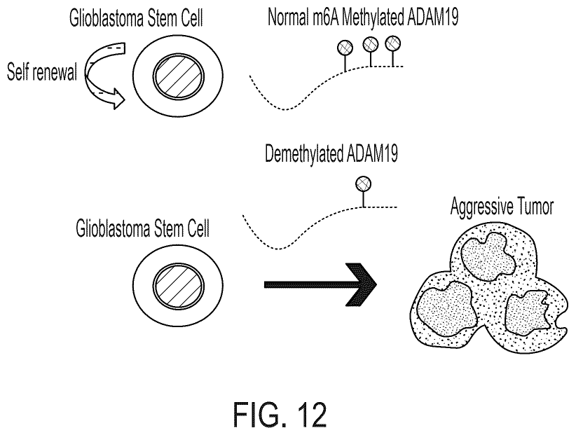

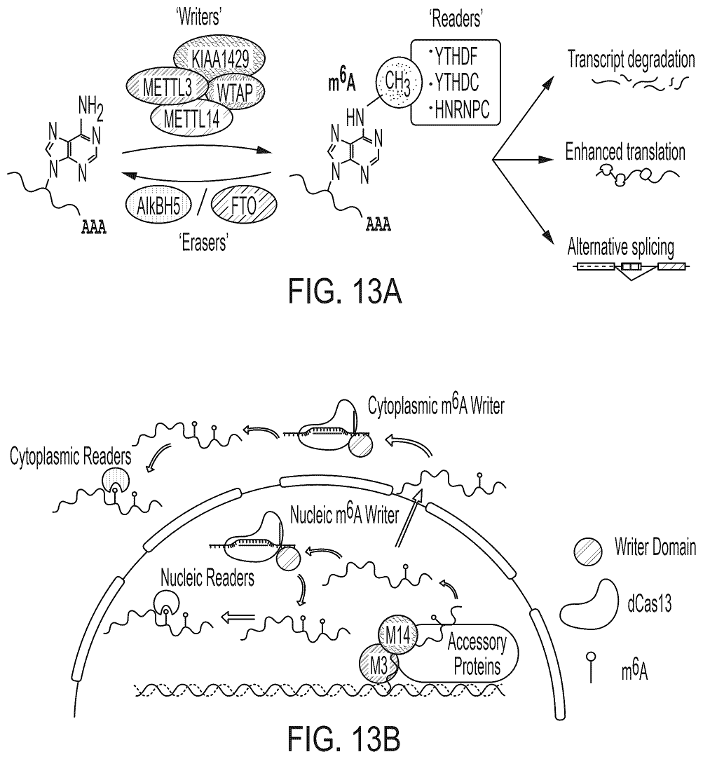

A recombinant Escherichia coli containing 2 vectors was constructed. One vector expresses Cas13b fused to METTL3 under an IPTG-inducible T7 promoter, followed by a constitutively expressed guide RNA. The guide RNA contains both a hairpin loop, necessary for proper binding to Cas13b, and an easily exchangeable (via golden gate cloning) spacer, which will allow for programmable targeting. The second vector contains an inducible target RNA, containing interspersed methylation sites on either side of a targeting area (see FIG. 11). The methylation sites are interspersed to cover a range of possible methylation locations. To test for the ability of the fusion proteins to perform targeted methylation activity in a medium-throughput manner, cultures of E. coli were grown under inducing conditions, non-inducing conditions, and with only the vector containing the target substrate in BL21 (DE3) Tuner cells. Total RNA was isolated and split-one half undergoing RT-qPCR using primers specific for the target substrate directly after isolation from the cells to serve as a control. The other half was subjected to an m⁶A enrichment via a commercially available m⁶A antibody conjugated to Dyna magnetic beads. See FIG. 7. The enriched methylated RNA was then subjected to RT-qPCR. Enrichment of the target construct (i.e., via a decrease in the threshold cycle (Ct) when compared to the control), confirms that expression of the fusion construct results in additional methylation of the target RNA. Only dCas13b-METTL3 expressed with IPTG showed significant enrichment of the target substrate indicating that it is possible to programmably target the substrate. It is believed that this system will allow for quick modifications and screening fusion constructs to optimize methyl transferase performance.

### Part 4. Off-Target Screen

Targeted methylation not only requires efficient methylation the target RNA, but also that the target is specifically methylated without changing the methylation state of background mRNAs. There are 2313 METTL recognition sites in the E. coli transcriptome. To test constructs for specificity, positive targeting complexes will be subjected to MeRIP-Seq ^{[8]} in the E. coli background. Total RNA will be isolated from E. coli cells in which Cas13b-METTL3 was expressed, and ribosome depletion will then be used to remove ribosomal RNA, which contain a large number of m⁶A sites. Samples will be sheared to 100mers and split, with one half being pulled down with m⁶A antibody conjugated to magnetic beads and the other half remaining untreated. Samples will then be subjected to reverse transcription and indexing and run on an inhouse NextSeq 550 sequencer (Illumina). Comparison of the control pool to the immunoprecipitation pool will allow for determination of significantly methylated sites as previously demonstrated ^{[8, 27]}. If the Cas13b-METTL3 fusion construct is specific for the target mRNA, then trivial differences should be observed between enriched METTL sites when comparing data from induced and non-induced pools.

### Construction of the “Eraser”

This section seeks to create a programmable m⁶A eraser for guided demethylation of target RNA sites. This section will employ a strategy similar that described in the above section for constructing the writer. First, a Km crippled version of the eraser will be constructed using available crystal structures and a computationally docked to an RNA target, and screen for mutants with an increased Km in vitro. Next, targeted demethylation will be validated using a known methylated target mRNA in yeast. Finally, off-target effects will be determined by MeRIP-Seq in yeast.

### Part 1: Creation of Binding Impaired ALKBH5

Unlike the large complex making up the native writer, m⁶A demethylation is performed by small monomers in the cell, FTO and ALKBH5. Crystal structures of ALKBH5 have been solved ^{[28-30]}, but none are co-crystallized with an RNA target. To ameliorate this problem and design possible mutations that weaken RNA binding, the ALKBH5 crystal structure was structurally aligned to a homolog of ALKBH5, ABH2, which is bound to dsDNA (PDB-3BUC). Then, one strand of the DNA was deleted and converted the remaining strand of DNA to RNA. The RNA sequence was then trimmed and changed to the canonical GGACU sequence using the software package 3DNA ^{[31]}. This model structure was then prepped with GROMACS and subjected to 100 ns of molecular dynamics simulation using the AMBER software package ^{[32]}. The resulting relaxed RNA bound model was examined for contacts between the RNA and the ALKBH5 structure. Rational decisions on possible mutations were compared to an in silico alanine scan of the binding surface using the Rosetta software package ^{[33]}. Eight possible mutations were identified that are intended to be screened for an increase in Km. The screen will be performed using the radioactive method described in the section relating to constructing the writer, above, with the caveat that the biotinylated RNA substrate will first be incubated with purified METTL3/14 complex. The addition of active ALKBH5 mutants will result in a reduction in the number of counts detected.

### Part 2: Screen of Cas13b-ALKBH5 Fusion for Activity In Vivo

Although bacteria have native m⁶A methylation, the RNA does not contain the canonical GGm⁶ACU demethylation motifs recognized by ALKH5B. To screen for in vivo demethylation activity, yeast will be used, which have well-described GGm⁶ACU methylated sites ^{[34]}. AMP deaminase (AMD1) will be targeted, however, because the optimum distance from the protospacer which would allow for demethylation is not yet known, tiled guide RNAs will be used to determine the optimum distance. A similar technique as that described above in the section relating to the writer construction will be used, which includes extracting total RNA, enrich for poly-A, and then perform RT-qPCR using primers targeting AMD1. In this case, de-enrichment following m⁶A immunoprecipitation will be targeted, indicated by a shift by an increase in cycle number (Ct). This setup will allow for medium-throughput optimization of various parts of the eraser construct.

### Part 3: Off Target Screen

Following successful demethylation of AMD1, MeRIP-Seq will be employed to determine off-target demethylation activity. There are 4096 m⁶A methylated sites in the yeast transcriptome. To determine the off-target demethylation, MeRIP-Seq will be performed on yeast that have the eraser present and those that don't (induced versus non-induced) and compare the 4069 sites to see if any have been unintentionally

### Validation in Cancer Cells

### Part 1: Targeting the ADAM19 Methylation State

Glioblastoma is one of the most aggressive forms of primary brain tumor. Currently, treatment includes surgery, chemotherapy, and radiation with little hope of survival after 15 months ^{[35]}. The disintegrin and metalloproteinase 19 (ADAM19) exhibit elevated expression in glioblastoma cells ^{[16]}. This increase in expression has been linked to depletion of m⁶A at 3′ UTR sites (see FIG. 11). The writer constructed herein may be used to add methyl groups to the 3′ UTR of ADAM19 in human cell lines and the eraser constructed herein will be used to remove methyl groups from the 3′ UTR of ADAM19, validating the ability of the system to add and remove methyl groups to this therapeutically important target.

### Part 2: Targeting the NANOG Methylation State

NANOG is a transcription factor thought to be a key player in maintaining stem cell pluripotency. In breast cancer, NANOG's m⁶A levels are diminished, promoting their stability and increasing expression ^{[36]}. This has been shown to contribute to the reacquisition of breast cancer stem cells ^{[36]}. The writer and eraser constructed herein may be used to add and remove methyl groups to NANOG, validating the ability of the systems to alter the m⁶A methylation state of this important target to human health.

### REFERENCES (CITED IN EXAMPLE 1)

1. Goldberg, A. D., C. D. Allis, and E. Bernstein, Epigenetics: a landscape takes shape. Cell, 2007. 128(4): p. 635-8.
2. Pawson, T. and J. D. Scott, Protein phosphorylation in signaling—50 years and counting. Trends Biochem Sci, 2005. 30(6): p. 286-90.
3. Pickart, C. M. and M. J. Eddins, Ubiquitin: structures, functions, mechanisms. Biochim Biophys Acta, 2004. 1695(1-3): p. 55-72.
4. Hoernes, T. P. and M. D. Erlacher, Translating the epitranscriptome. Wiley Interdiscip Rev RNA, 2017. 8(1).
5. Meyer, K. D. and S. R. Jaffrey, The dynamic epitranscriptome: N6-methyladenosine and gene expression control. Nat Rev Mol Cell Biol, 2014. 15(5): p. 313-26.
6. Engel, M. and A. Chen, The emerging role of mRNA methylation in normal and pathological behavior. Genes Brain Behav, 2017.
7. Alarcon, C. R., et al., N6-methyladenosine marks primary microRNAs for processing. Nature, 2015. 519(7544): p. 482-5.
8. Meyer, K. D., et al., Comprehensive analysis of mRNA methylation reveals enrichment in 3′ UTRs and near stop codons. Cell, 2012. 149(7): p. 1635-46.
9. Choi, J., et al., N(6)-methyladenosine in mRNA disrupts tRNA selection and translation-elongation dynamics. Nat Struct Mol Biol, 2016. 23(2): p. 110-5.
10. Zhou, J., et al., Dynamic m(6)A mRNA methylation directs translational control of heat shock response. Nature, 2015. 526(7574): p. 591-4.
11. Liu, N., et al., N(6)-methyladenosine-dependent RNA structural switches regulate RNA-protein interactions. Nature, 2015. 518(7540): p. 560-4.
12. Aguilo, F., et al., Coordination of m(6)A mRNA Methylation and Gene Transcription by ZFP217 Regulates Pluripotency and Reprogramming. Cell Stem Cell, 2015. 17(6): p. 689-704.
13. Chen, T., et al., m(6)A RNA methylation is regulated by microRNAs and promotes reprogramming to pluripotency. Cell Stem Cell, 2015. 16(3): p. 289-301.
14. Xiang, Y., et al., RNA m⁶A methylation regulates the ultraviolet-induced DNA damage response. Nature, 2017. 543(7646): p. 573-576.
15. Jaffrey, S. R. and M. G. Kharas, Emerging links between m⁶A and misregulated mRNA methylation in cancer. Genome Med, 2017. 9(1): p. 2.
16. Cui, Q., et al., m⁶A RNA Methylation Regulates the Self-Renewal and Tumorigenesis of Glioblastoma Stem Cells. Cell Rep, 2017. 18(11): p. 2622-2634.
17. Liu, J., et al., A METTL3-METTL14 complex mediates mammalian nuclear RNA N6-adenosine methylation. Nat Chem Biol, 2014. 10(2): p. 93-5.
18. Sledz, P. and M. Jinek, Structural insights into the molecular mechanism of the m(6)A writer complex. Elife, 2016. 5.
19. Zheng, G., et al., ALKBH5 is a mammalian RNA demethylase that impacts RNA metabolism and mouse fertility. Mol Cell, 2013. 49(1): p. 18-29.
20. Komor, A. C., et al., Programmable editing of a target base in genomic DNA without double-stranded DNA cleavage. Nature, 2016. 533(7603): p. 420-4.
21. Gaudelli, N. M., et al., Programmable base editing of A*T to G*C in genomic DNA without DNA cleavage. Nature, 2017.
22. Cox, D. B. T., et al., RNA editing with CRISPR-Cas13. Science, 2017.
23. Liu, X. S., et al., Editing DNA Methylation in the Mammalian Genome. Cell, 2016. 167(1): p. 233-247 e17.
24. Wang, X., et al., Structural basis of N(6)-adenosine methylation by the METTL3-METTL14 complex. Nature, 2016. 534(7608): p. 575-8.
25. Dorgan, K. M., et al., An enzyme-coupled continuous spectrophotometric assay for S-adenosylmethionine-dependent methyltransferases. Anal Biochem, 2006. 350(2): p. 249-55.
26. Li, F., et al., A Radioactivity-Based Assay for Screening Human m⁶A-RNA Methyltransferase, METTL3-METTL14 Complex, and Demethylase ALKBH5. J Biomol Screen, 2016. 21(3): p. 290-7.
27. Deng, X., et al., Widespread occurrence of N6-methyladenosine in bacterial mRNA. Nucleic Acids Res, 2015. 43(13): p. 6557-67.
28. Aik, W., et al., Structure of human RNA N(6)-methyladenine demethylase ALKBH5 provides insights into its mechanisms of nucleic acid recognition and demethylation. Nucleic Acids Res, 2014. 42(7): p. 4741-54.
29. Feng, C., et al., Crystal structures of the human RNA demethylase Alkbh5 reveal basis for substrate recognition. J Biol Chem, 2014. 289(17): p. 11571-83.
30. Xu, C., et al., Structures of human ALKBH5 demethylase reveal a unique binding mode for specific single-stranded N6-methyladenosine RNA demethylation. J Biol Chem, 2014. 289(25): p. 17299-311.
31. Leaver-Fay, A., et al., ROSETTA3: an object-oriented software suite for the simulation and design of macromolecules. Methods Enzymol, 2011. 487: p. 545-74.
32. Bodi, Z., et al., Yeast m⁶A Methylated mRNAs Are Enriched on Translating Ribosomes during Meiosis, and under Rapamycin Treatment. PLoS One, 2015. 10(7): p. e0132090.

Each of the references 1-32 cited in Example 1 are herein incorporated by reference in their entireties as forming a part of the original filed disclosure.

### Example 2. Programmable Epitranscriptome Editors

### Results

In-vitro characterization of m⁶A methyltransferase. Hypothetical dCas13-targeted m⁶A editors face the challenge of specificity—ensuring m⁶A methylation occurs only at desired sites. To address this problem, it was thought that weakening the substrate affinity (Kₘ) while preserving the catalytic rate (K_{cat}) of an m⁶A methyltransferase could make its activity dependent on dCas13 RNA targeting. The increase in local concentration from dCas13-RNA binding would overcome this Kₘ impairment only at the intended RNA target, thereby providing specificity.

Crystal structures of the METTL3/METTL14 core m⁶A writer complex suggest that METTL3 functions as the active methyltransferase while METTL14 facilitates RNA substrate binding at the appropriate DRACH sequence motif (D=A, G or U; R=A or G; H=A, C, or U)^{24, 33}. As only METTL3 contains a properly arranged SAM-dependent active site, it was hypothesized that METTL3 could methylate m⁶A on its own and serve as a binding-impaired methyltransferase fusion to dCas13 for programmable m⁶A writing.

To test this, Michaels-Menten kinetics of the METTL3/METTL14 complex were compared to METTL3 alone using a radiometric assay which monitors the transfer of C¹⁴ from SAM cofactor to an RNA substrate³⁴. To further ablate the RNA binding affinity of METTL3, the zinc finger RNA-binding motifs were removed from METTL3.

Although the Vₘₐₓ of METTL3 was modestly reduced in the absence of METTL14, its Kₘ was severely impaired (Table 1). Thus, M3 may be the ideal fusion to Cas13 as the increase in local concentration provided by Cas13 binding to the targeted transcript can overcome the elevated Kₘ of M3 alone and provide specificity for the target RNA. In addition, removal of the zinc finger RNA-binding motifs from M3 may make the M3/M14 complex reliant on Cas13b binding for efficient turnover.

TABLE 1

| Construct | Km[nM] | Vₘₐₓ (h⁻¹) |
|---|---|---|
| METTL3/METTL14 | 22 +/− 3 | 23 +/− 4 |
| METTL3 | >900 | 4 +/− 2 |

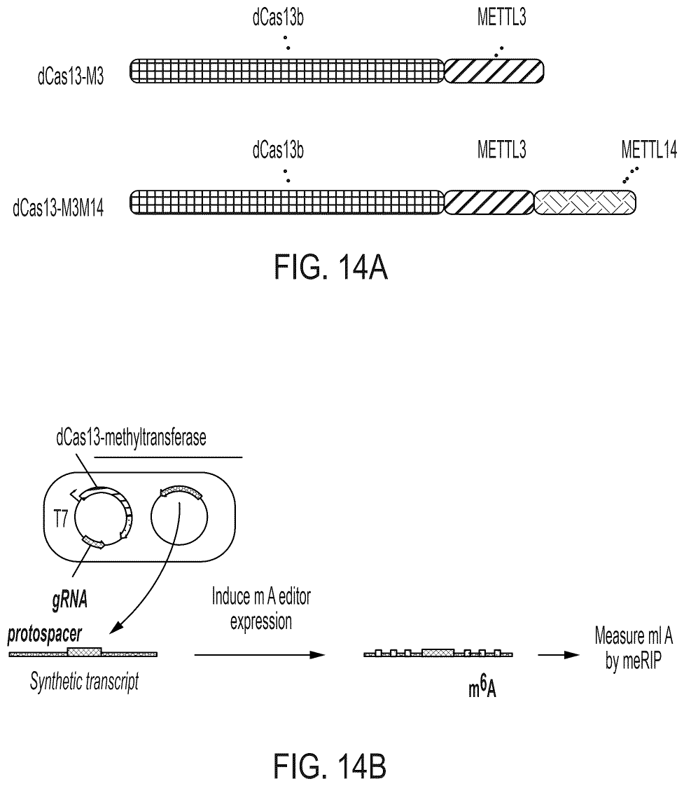

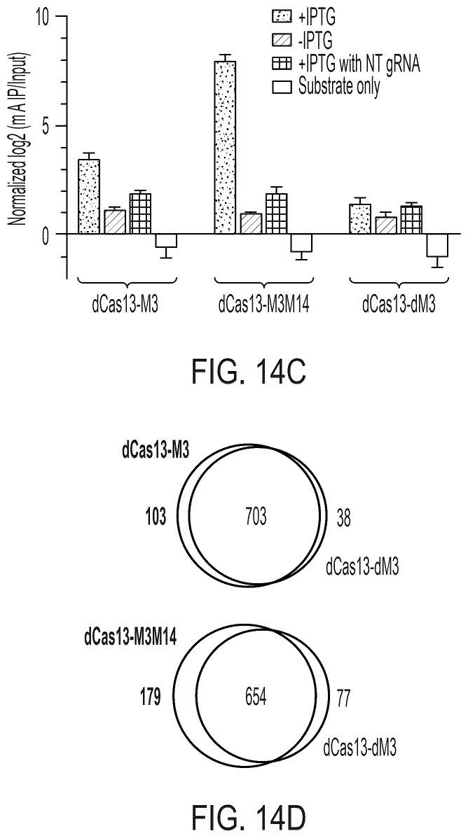

Design of the fusion Cas13 Fusion protein. Next, in order to construct programmable m⁶A writers, candidate m⁶A methyltransferases were fused to dCas13 and a nuclear localization sequence or nuclear export sequence. Due to its high expression and activity, a truncated form of the Cas13b variant from Prevotella sp. P5-125 (PspCas13b Δ984-1090) was elected and its HEPN nuclease domain was inactivated with an active-site mutation (H133A)³². To design fusions of METTL3 and METTL3/METTL14 to dCas13, a previously published crystal structure of PbuCas13b (PDB 6DTD), a homolog of PspCas13b (˜40% sequence similarity), ^{35, 36} was examined. Although its N-terminus is buried within the protein core, its C-terminus is accessible on the surface. Therefore, candidate m⁶A methyltransferases were tethered exclusively to the C-terminus of dCas13. In addition, inspection of the METTL3/METTL14 heterodimer structure bound to SAM³³ (PDB 5IL1) revealed that the dimer conformation would be hindered by a Cas13-M14-linker-M3 architecture. Thus, it was elected to move forward with four dCas13-M3M14 and dCas13-M3 m⁶A editor constructs that had been generated: M3nls, M3nes, M3M14nls, and M3M14nls (FIG. 14A).

Validation of m⁶A editing in bacteria. To test whether the constructs could specifically methylate RNA in a cellular context, a demonstration of m⁶A editing in bacterial cells was sought. Recombinant Escherichia coli were constructed with a vector expressing dCas13-methyltransferase fusions under an IPTG-inducible promoter, followed by a constitutively-expressed gRNA. A second vector encoded a synthetic target transcript containing m⁶A methylation sites (GGACU) arrayed around a gRNA-targeting sequence (FIG. 14B). To measure m⁶A modification of the targeted transcript, RT-qPCR was used to quantify enrichment of RNA fragments immunoprecipitated with m⁶A antibodies (meRIP-RT-qPCR). Within this bacterial system, significant m°A methylation of the target substrate only upon induction of dCas13-M3 and dCas13-M3M14 expression (FIG. 14C) was observed. Furthermore, methyltransferase activity was required for editing, as a methyltransferase-impaired dCas13-M3 D395A (dCas13-dM3) caused negligible m⁶A enrichment. Lastly, expression of active dCas13-methyltransferases with non-targeting gRNA resulted in minimal target methylation, showing that gRNA targeting was necessary for specific editing at desired sites. Collectively, these findings demonstrate the ability to selectively methylate intended RNA targets with both dCas13-M3 and dCas13-M3M14. This bacterial system may allow for quick modifications of screen fusion constructs to further optimize the m⁶A editor's performance in future studies.

Targeted methylation not only requires efficient methylation of the intended RNA, but also high specificity for the target to ensure minimal perturbation of background mRNAs. Overexpression of methyltransferases in a cellular context could unintentionally increase methylation of off-target mRNA. To evaluate the specificity of m⁶A editor constructs expressed in E. coli, cellular RNA was extracted and fragmented, and then the RNA enriched with m⁶A antibodies (meRIP-seq)¹² was sequenced. There are 2,313 METTL recognition sites in the E. coli transcriptome. Though 806 methylated m⁶A sites in E. coli which expressed dCas13-M3 were found, and 103 were not present in a methyltransferase-inactive dCas13-dM3 control. Similarly, 179 out of 833 methylated m⁶A sites were found in the dCas13-M3M14 condition, but not an inactive editor control (FIG. 14D). These results suggest modest off-targeting from both dCas13-M3 and dCas13-M3M14 constructs.

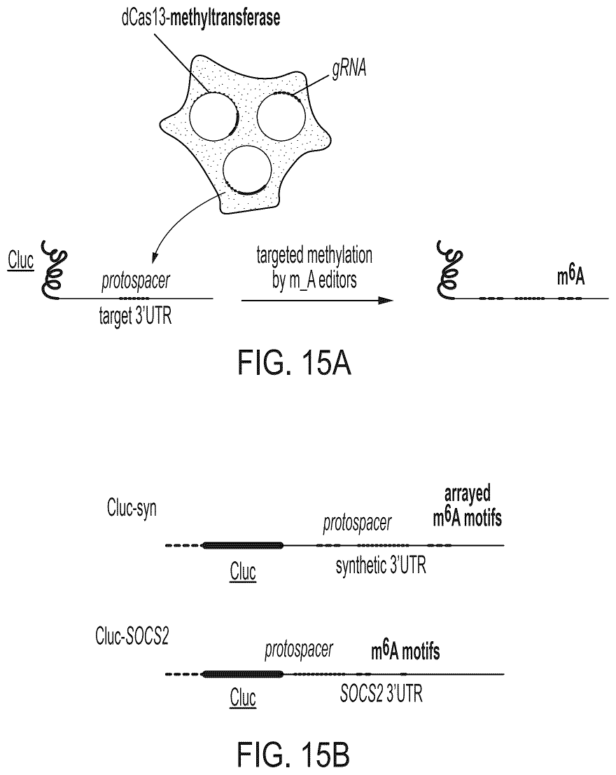

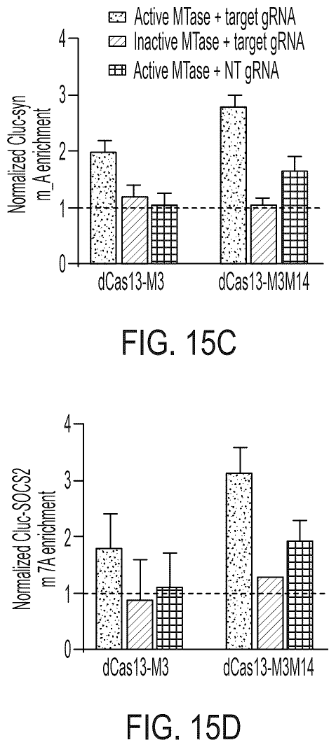

Methylation of Reporter Transcripts in Mammalian Cells. To assess whether m⁶A editors can induce site-specific m⁶A modification in human cells, gRNAs targeting a synthetic RNA substrate placed on the 3′ UTR of Cypridina luciferase (Cluc) mRNA were designed. Then, m⁶A sites arrayed around this reporter's spacer sequence were targeted with dCas13-methyltransferase fusions transfected in HEK293T cells (FIG. 15A-15B). MeRIP-RT-qPCR of this arrayed reporter (Cluc-syn) revealed increased methylation from reporter-targeted dCas13-M3 and dCas13-M3M14, but none from methyltransferase-inactive constructs (FIG. 15C). A small increase in m⁶A modification from dCas13-M3M14 with a non-targeting gRNA was also found, indicating modest off-target methylation from this construct. Notably, off-target methylation from dCas13-M3 was not observed, suggesting that the Km-impaired METTL³²⁷³⁻⁵⁸⁰ possesses reduced gRNA-independent activity. The same trend was observed, and the findings confirmed, with a second reporter transcript in which the endogenous 3′ UTR of the Suppressor of cytokine signaling (SOCS2) gene was fused onto Cluc (FIG. 15D).

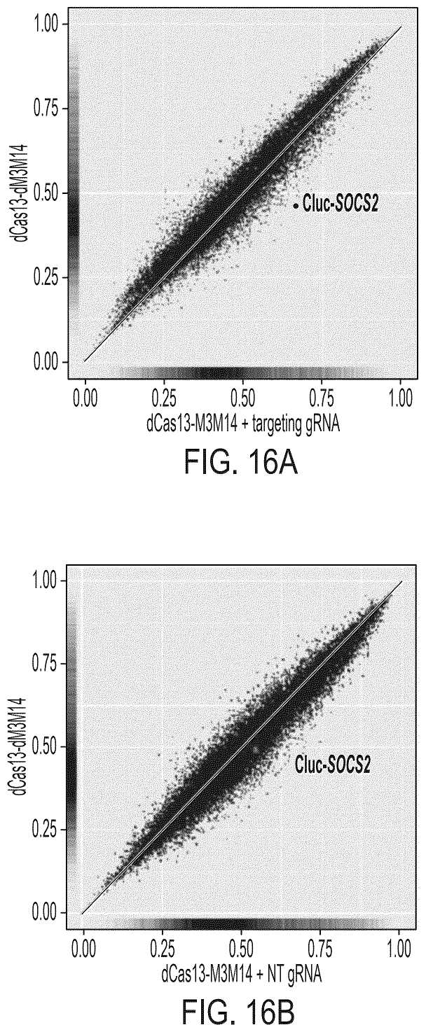

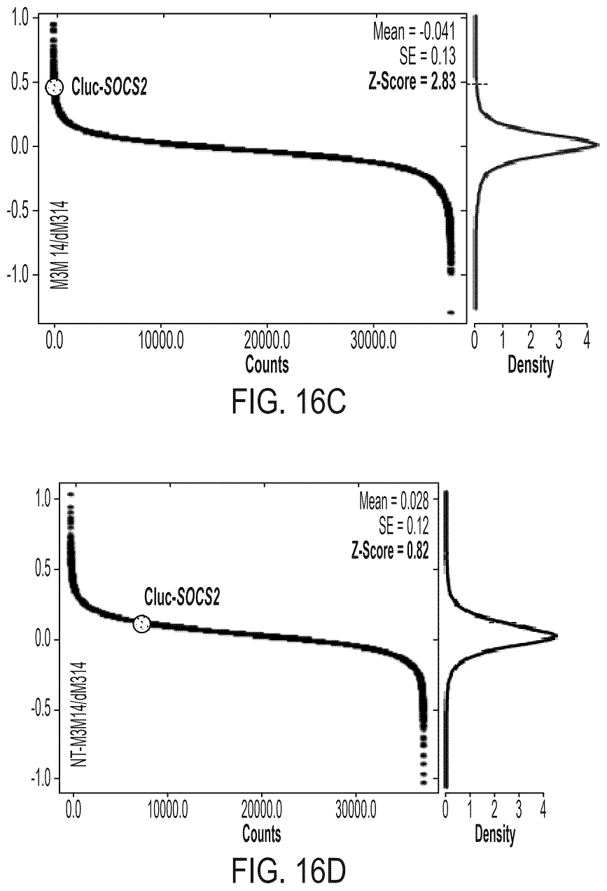

To further explore on-target and off-target methylation activity, meRIP-seq of the m⁶A editors was performed, targeting the Cluc-SOCS2 reporter. In agreement with RT-qPCR results, meRIP-seq traces of Cluc-SOCS2 reveal increased m⁶A levels only with methyltransferase-active and reporter-targeting m⁶A editors. Plotting differential m⁶A methylation of the entire transcriptome, it was observed that reporter-targeted dCas13-M3 and dCas13-M3M14 promoted an increased methylation of Cluc-SOCS2 over background (FIGS. 16A-16B). The mean of these rank plots is close to zero.

Rank ordering these transcriptome-wide m⁶A sites revealed that this reporter was selectively methylated, with dCas13-M3M14, yielding a Z-score of 2.83 for the log 2 (FC) distribution (FIG. 16C). In contrast, an 0.82 Z-score comparing non-targeted dCas13-M3M14 with a methyltransferase-inactive control was found, indicating minimal gRNA-independent alteration of background methylation states (FIG. 16D).

Only 237 of 37,000 background (i.e. possible) adenosine methylation locations in the Cluc-SOCS2 target exhibited higher m⁶A enrichment than the target location (FIGS. 16C and 16D). That translates to an off-target modification frequency of 0.64%. As a whole, this demonstrates that the dCas13-methyltransferase fusions can install m⁶A on exogenous reporter RNAs in human cells, with high RNA modification efficiencies.

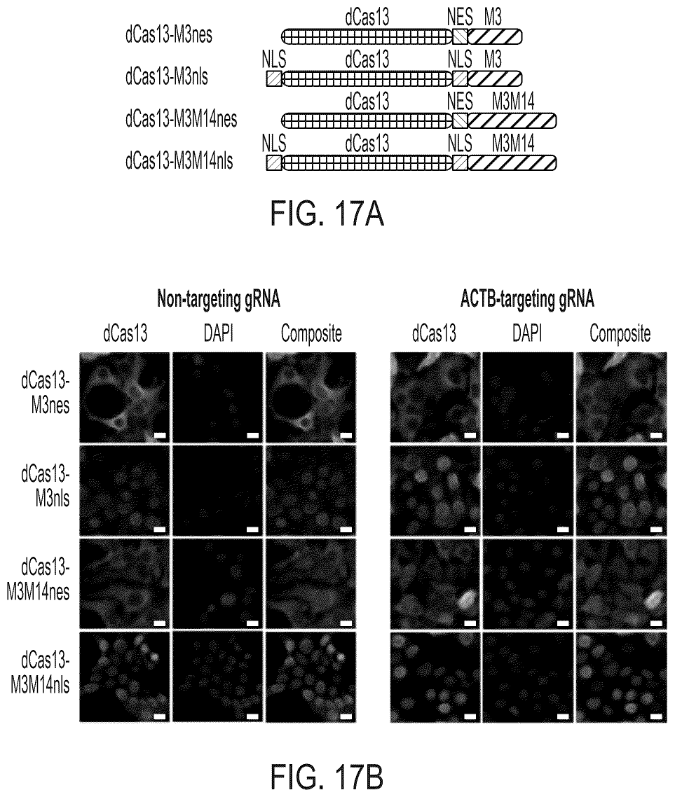

Engineered cytoplasmic and nucleus localized m⁶A-editors. As human m⁶A readers exist within both the cytoplasm and nucleus, it was reasoned that it may be advantageous for researchers to have m⁶A editors localized to each cellular compartment. Thus, nuclear- and cytoplasmic-localized variants of each construct were engineered by placing NES and NLS sequences in the linker of each, generating dCas13-M3nes, dCas13-M3nls, dCas13-M3M14nes, and dCas13-M3M14nls (FIG. 17A). To confirm the intracellular localization of these editors, C-terminal 3× hemaglutinin (HA) epitopes were cloned onto each construct, immunostained, and transfected in HEK293T cells. As expected, all NES-tagged m⁶A editors localized in the cytoplasm, while all NLS-tagged editors localized in the nucleus (FIG. 17B). Next, to investigate whether RNA targeting affects editor localization, constructs were visualized with non-targeting gRNA or gRNA targeting beta-actin (ACTB), a highly abundant transcript which predominantly resides in the cytoplasm. It was found that all m⁶A editors were localized to their intended cellular compartments, suggesting insignificant co-export of nuclear-localizing constructs with ACTB transcripts (FIG. 17B). Cells transfected with targeting or non-targeting gRNA showed comparable viability and morphology, indicating that Cas13b had no apparent effect on cell survival or morbidity. Therefore, it was determined that the intracellular localization of the m°A editor could be controlled with fused localization tags.

To explore the possibility that dCas13 on its own may alter RNA, the effects of dCas13 binding on transcript stability and translation were investigated. First, a dual-luciferase reporter vector was constructed expressing a Cluc target transcript and a Gaussia luciferase (Gluc) internal dosing control. Then HEK293T cells were transfected with NES- and NLS-tagged dCas13, Cluc-targeting gRNAs, and dual-luciferase vector 48 hours before measuring luciferase RNA abundance and expression.

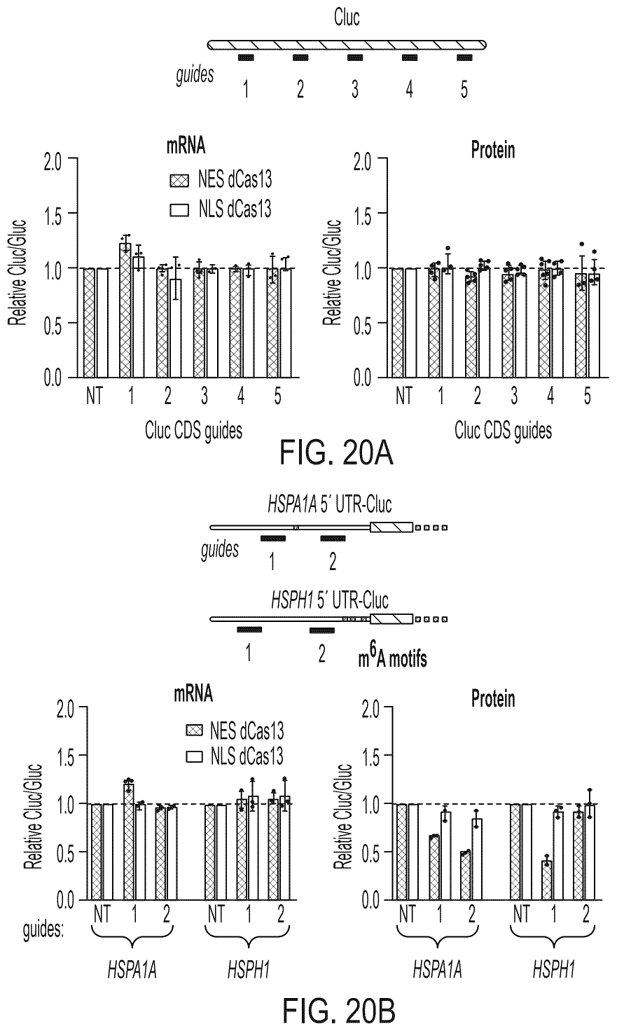

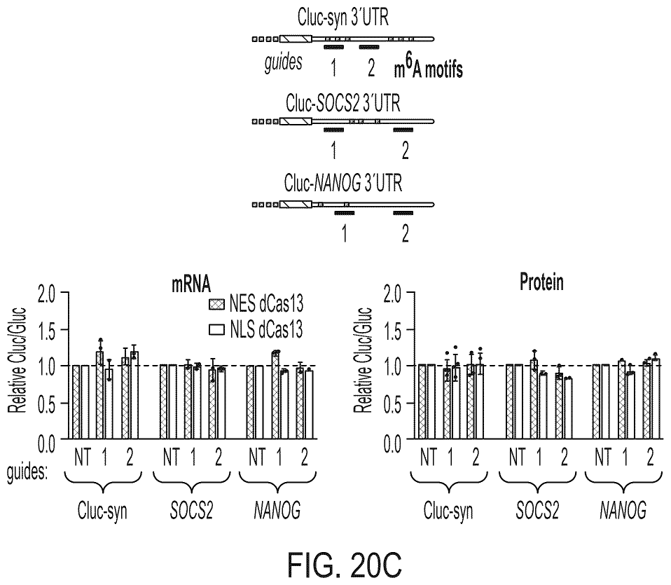

Using guides tiling the Cluc coding region, it was found that dCas13 binding did not significantly affect Cluc RNA and protein amounts (FIG. 20A). As m⁶A modifications commonly reside within the un-translated regions (UTRs) of mRNA, next the UTRs of Cluc reporters harboring 5′ and 3′ UTRs with m⁶A sites were targeted. A minimal alteration of Cluc RNA abundance or expression was observed when dCas13 was directed to the 3′ UTRs of the synthetic arrayed reporter (Cluc-syn), Cluc-SOCS2, and Cluc-NANOG (FIG. 20B). In contrast, targeting the 5′UTRs of HSPA1A-Cluc and HSPH1-Cluc reporters resulted in up to a 60% decrease in Cluc protein expression, but not RNA abundance (FIG. 20C). Only cytoplasmic-localized dCas13 showed this effect, suggesting that 5′UTR binding may interfere with ribosome scanning and RNA translation. Taken together, this reveals that dCas13 targeting minimally perturbs RNA abundance and only reduces translation efficiency at 5′UTRs on mRNA within in the cytoplasm. These data also suggest that a nucleus-localized m⁶A editor would be superior to a cytoplasm-localized or untagged editor because the nucleus-localized editor would be able to target RNA 5′ UTRs without an unintended drop in translation efficiency.

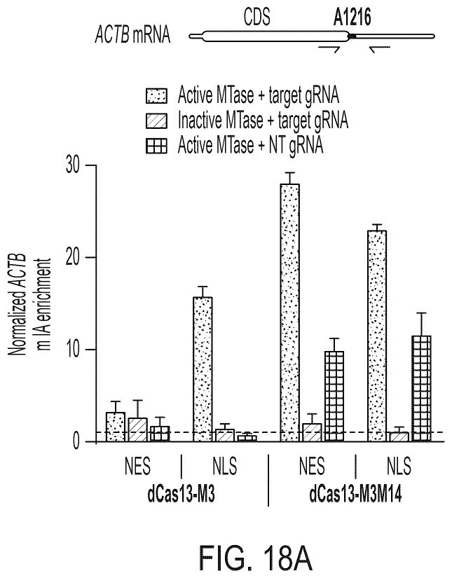

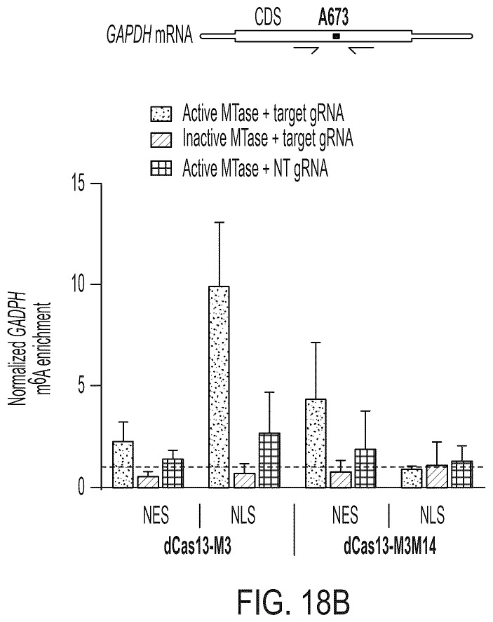

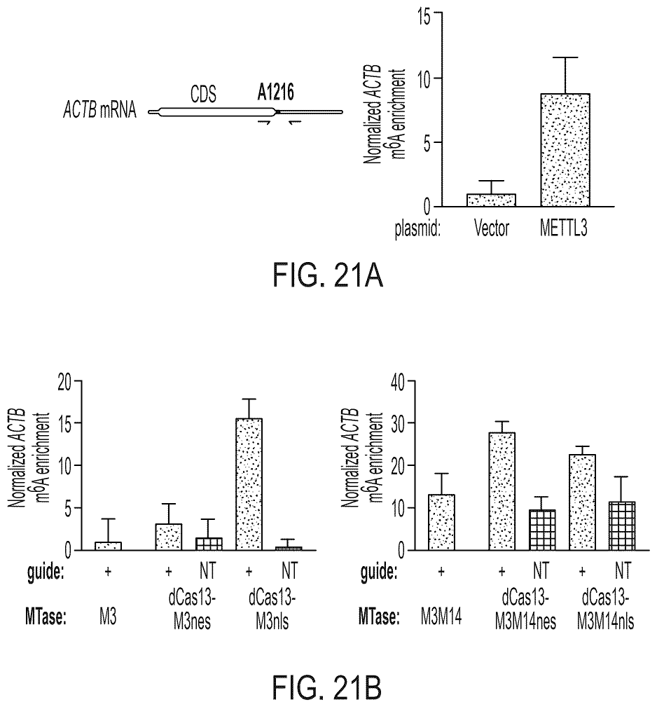

Endogenous Transcript Targeting with m⁶A-Writer. Next, with the suite of cytoplasmic- and nuclear-localizing editors, m⁶A modifications on endogenous transcripts in HEK293T cells were installed. First, the A1216 locus was targeted on the beta-actin (ACTB) mRNA, which is methylated at low frequencies in HEK293T cells³⁷. As modification at this site is sensitive to overexpression of METTL3, ACTB A1216 methylation was regulated by canonical m⁶A writers (FIG. 21A). ACTB methylation was measured by the four constructs under three conditions: methyltransferase-active editor with an ACTB-targeting guide, inactive editor, and active editor with a scrambled non-targeting guide compared to an empty vector control. It was found that dCas13-M3nls and both M3M14 constructs could install m⁶A at ACTB A1216 (FIG. 18A). However, the M3M14 constructs induced modest target methylation when supplied with a non-targeting guide, whereas dCas13-M3nls did not. This data suggests that dCas13-M3nls has less guide-independent off-target methylation activity than constructs containing M3M14. In addition, it was evaluated whether a targeting guide with the M3 or M3M14 methyltransferase domains alone could stimulate dCas13-independent methylation. No increase in ACTB methylation above control was found (FIG. 21B). To confirm the results obtained for ACTB, the editors were used to target another endogenous transcript, Glyceraldehyde 3-phosphate dehydrogenase (GAPDH) at A673, which was unmethylated in HEK293T cells. As with ACTB, significant methylation of GAPDH was observed only in the presence of a targeting guide RNA (FIG. 18B). Although dCas13-M3nls effectively installed m⁶A, M3M14 constructs methylated with lower efficiency at GAPDH A673.

Next, it was determined whether similar effects could be achieved with lower levels of editors to confirm that the methyl group was being added to the suspected adenine using an orthogonal method that does not require antibody enrichment. To do this, a new method for direct m⁶A profiling was used that takes advantage the inability for MazF to cleave at adenines containing m⁶A marks. ³⁸

MeRIP-seq was further performed on the conditions above. To evaluate the extent of off-targeting, meRIP-seq was performed under the two different editor amounts described above and compared to both an active dCas13-writer target, and an active writer with a scrambled, non-targeting guide NT-dCas13b-writer to catalytically dead fused writer dCas13-dwriter. Comparison of the amount of methylation.

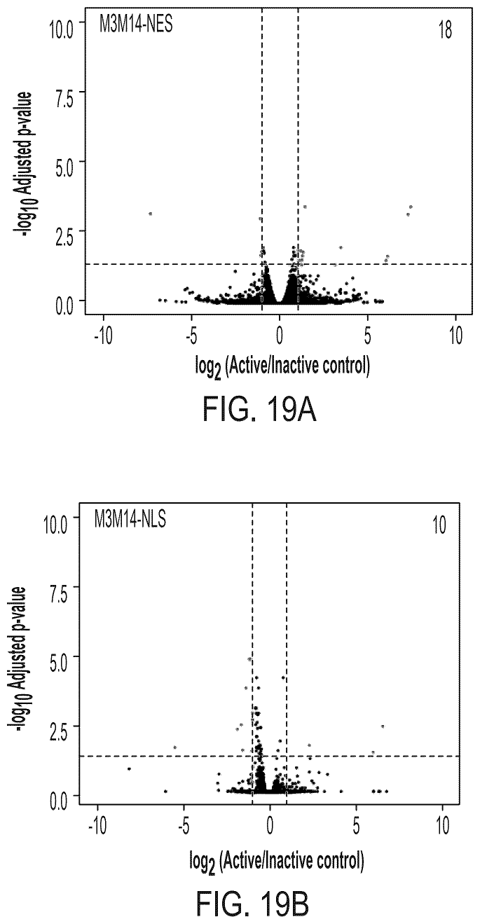

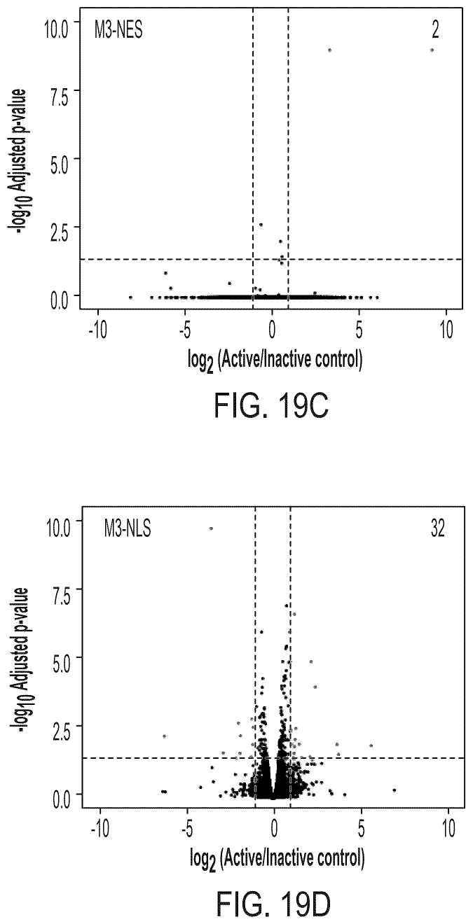

Endogenous gene differential RNA-seq. One of the major phenotypic effects of adenine methylation is to increase or decrease the expression of methylated transcripts. To test the effect of the editors on RNA expression transcriptome-wide, differential RNA-seq analysis was performed with all four versions of the editors. It was found that differential RNA-seq adopted the following order for number of differentially expressed transcripts—M3_NES<<M3/M14_NLS<M3/M14_NES<M3_NLS (FIG. 19A-19D).

### Discussion

Since the central dogma was first proposed in 1957 by Francis Crick ³⁹, increasing layers of complexity have been gradually added at the DNA, RNA, and protein levels. Post-transcriptional modification of proteins and methylation of DNA have been well studied, while the role of post-translational modification of RNA has only recently been discovered. By coupling Cas13 with m⁶A RNA writers, the present disclosure offers researchers the first step towards a versatile toolbox to manipulate the epitranscriptome. With this and similar editors, it is now possible to achieve site-specific m⁶A installation, which is paramount to establishing cause-effect relationships between individual sites and the effect of m⁶A methylation on phenotype.

Here, a Cas13-based methylation writing system capable of installing the m⁶A modification was developed. Cas13 may be especially beneficial for two primary reasons—it requires a single guide with no sequence context, and it retains its ability to process its CRISPR array, allowing easy multiplexing. Multiplexing allows researchers to target dozens or more sites in a single experiment.

This is viewed as one of the first steps towards developing a tool kit for RNA researchers to manipulate the RNA transcriptome which may allow for the building of editors with higher efficiency, better specificity, and increased complexity.

Table 2 shows a list of the guide RNA sequences used for experiments in HEK293 cells.

TABLE 2

| Target RNA | FIG.(S) | Cell type | Guide RNA protospacer |
|---|---|---|---|
| Non-targeting | 15A-15D, 16A- | HEK293T | GTAATGCCTGGCTTGTCGACGCATAGTCTG |
| | 16D, 17A-17B, | | (SEQ ID NO: 29) |
| | 18A-18B, 19A- | | |
| | 19D | | |
| Cluc-syn 3′ | 15A-15D, 16A- | HEK293T | TTCCAAACTATCCTGCGGCCTCTACTCTGC |
| UTR reporter | 16D | | (SEQ ID NO: 30) |
| Cluc-SOCS2 3′ | 15A-15D, 16A- | HEK293T | TACATAGCTGCATTCGGAGATACTCTATGT |
| UTR erporter | 16D | | (SEQ ID NO: 31) |
| ACTB A1216 | 17A-17B, 18A- | HEK293T | GAAGCATTTGCGGTGGACGATGGAGGGGC |
| | 18B, | | C (SEQ ID NO: 32) |
| GAPDH A673 | 18A-18B | HEK293T | AGCCCCGCGGCCATCACGCCACAGTTTCCC |
| | | | (SEQ ID NO: 33) |

### Methods

General methods and molecular cloning. All new plasmids used in this study were assembled using Uracil-Specific Excision Reagent (USER) cloning. In this procedure, deoxyuracil-containing primers (Integrated DNA Technologies or Eton Biosciences) were used to amplify DNA fragments with Phusion U Green Multiplex PCR Master Mix (Thermo Fisher), using polymerase chain reaction (PCR). The PCR products were electrophoresed on a 1% agarose gel containing 0.015% ethidium bromide and imaged with a G:Box gel imager (Syngene) to confirm their identity. DNA fragments with deoxyuracil incorporated near the 5′ ends were then assembled using USER Enzyme, CutSmart Buffer, and DpnI restriction enzyme (New England BioLabs), per manufacturer's protocol. One Shot™ Mach1 Chemically Competent E. coli cells (Invitrogen) were transformed with assembled plasmids and grown on carbenicillin-containing agar plates overnight. DNA from selected colonies was amplified with TempliPhi 100 Amplification Kit (Sigma-Aldrich) and Sanger sequenced (Quintara Biosciences) to confirm plasmid identity. Colonies containing correct plasmids were grown in 2×YT medium overnight, and plasmids were purified with either QIAprep Spin Miniprep Kit (Qiagen) or Zymopure II Midiprep Kit (when used for mammalian cell transfection, Zymo Research). DNA concentration and purity were determined using a NanoDrop Spectrophotometers (Thermo Fisher).

HEK cell culture, transfections. Immunofluorescence microscopy, MeRIP-seq, and RNA-seq were performed with HEK293T cells (American Type Culture Collection (ATCC)). Cells were grown in Dulbecco's Modified Eagle Medium with high glucose, GlutaMAX, and sodium pyruvate (Thermo Fisher), supplemented with 10% FBS (VWR) and 1× penicillin-streptomycin. Cells were passaged every 48 hours by diluting 1:5 with fresh culture media, in order to maintain confluency below 80%. Cells were transfected at 50% confluency for immunofluorescence microscopy and at 80% confluency for m⁶A addition assays. In both experiments, transfection plasmids were mixed with Opti-MEM I Reduced Serum Media (Thermo Fisher) to a total volume of 25 uL. Separately, 1 uL (for immunofluorescence microscopy) or 3 uL (for MeRIP-seq) of Lipofectamine 2000 (Thermo Fisher) were mixed with MEM I Reduced Serum Media to a total volume of 25 uL. The plasmid and lipofectamine solutions were then combined, incubated for 10 minutes at room temperature, and applied onto cells.

RNA isolation. Bacterial RNA lysis and isolation was performed using TRIzol (Thermo)+Max bacterial enhancement (Thermo). Mammalian RNA lysis was performed using TRIzol. Aqueous phase of the TRIzol preparation was added to a RNeasy column (Qiagen) to further clean and concentrate the RNA.

Immunofluorescence microscopy. A 3× hemagglutinin (3×HA) epitope tag (YPYDVPDYAYPYDVPDYAYPYDVPDYA (SEQ ID NO: 28)) was cloned into the C′ terminus of existing dCas13b m⁶A editors. HEK293T cells were grown on poly-D-lysine/laminin 12 mm coverslips (Corning) placed on 24-well plates. After confluency reached 50%, each coverslip was transfected with 250 ng 3×HA-tagged editor plasmid, 250 ng gRNA plasmid, and 25 ng Cluc-SOCS2 target plasmid, combined with 1 uL Lipofectamine 2000 (Thermo Fisher). After 36-48 hours of incubation at 37 degrees, culture media was aspirated, and coverslips were washed once with PBS for 2 minutes. Cells were fixed by incubating in 4% PFA (Electron Microscopy Sciences) for 30 minutes at room temperature in dark. Cells were then washed 3 times, 5 minutes each time, with PBS, and permeabilized by incubating in PBS+0.1% Triton (PBST) for 1 hour at room temperature. Cells were stained with a mouse anti-HA monoclonal primary antibody (Cell Signaling Technology, 2367) dissolved 1:100 in blocking buffer (3% BSA in PBST) for 12 hours at 4 degrees with shaking. Cells were then washed 5 times with PBST, 5 minutes each time, and stained for 1 hour at room temperature while shaking with a goat anti-mouse IgG, AF488 secondary antibody (Thermo Fisher, A-11029), dissolved 1:800 in blocking buffer. Cover slips were washed 3 times with PBST, 5 minutes each time, and 38 mounted onto microscope slides (VWR) with ProLong™ Diamond Antifade Mountant with DAPI (Invitrogen). Images were acquired using an Axioplan 2 fluorescence microscope (Carl Zeiss) and analyzed using MetaMorph and ImageJ software.

meRIP-sequencing data analysis. Total RNA was poly(A) enriched using Dynabeads Oligo (dT)25 (Thermo Fisher) and fragmented to a mean size of 200-300 nucleotides by incubation in 30 mM MgCl2 for 8 min at 95 degrees. Samples were incubated overnight at 4 degrees with protein G magnetic beads (Thermo Fisher) coated with EpiMark anti-m⁶A antibody (New England BioLabs). Washes and elution were performed on a Biomek liquid handler (Beckman Coulter). Samples were washed five times to remove unbound RNA with each of the following buffers: reaction buffer (150 mM NaCl, 10 mM Tris-HCl, pH 7.5, 0.1% NP-40 in nuclease-free H2O), low-salt reaction buffer (50 mM NaCl, 10 mM Tris-HCl, pH 7.5, 0.1% NP-40 in nuclease-free H2O), and high salt reaction buffer (500 mM NaCl, 10 mM Tris-HCl, pH 7.5, 0.1% NP-40 in nuclease-free H2O). RNA was eluted using RLT buffer (Qiagen) and purified with RNA Clean & Concentrator-5 kits (Zymo Research). RNA libraries were constructed using SMARTer PrepX Apollo NGS library prep system (Takara) following manufacturer's protocol. Libraries were normalized and ran on a NextSeq 550 sequencer (Illumina) using single read 75 cycle kit.

MeRIP-seq reads were aligned to the human transcriptome using HISAT2 (Johns Hopkins University) with reference annotation UCSC hg38. To facilitate reads coverage visualization and comparison between samples, UCSC tools and RSeQC were employed for BigWig format transforming and normalization separately. For transcriptome-based methylation detection, the R package (m⁶A monster) was used to bin and count reads.

RNA-sequencing data analysis. Sequencing libraries were prepared using poly(A)-enriched RNA on a SMARTer PrepX Apollo NGS library prep system (Takara) following manufacturer's protocols. Libraries were normalized and ran on a NextSeq 550 sequencer (Illumina). Trimmomatic (Usadel Lab) was used to exclude adaptor reads and low-quality reads. Reads were aligned to hg38 transcriptome with reference UCSC hg38 annotation by Kallisto (Pachter Lab). Sleuth pipeline (Pachter Lab) was used to quantify and normalize the mRNA expression levels. Results were visualized in R.

### Sequences

HIV Nuclear export signal=Bold; SV40 Nuclear localization signal=Bold and Italics; dPspCas13b Δ984-1090=Plain Text; GS linker=Underlined; 16aa XTEN linker=Bold and Underlined; METTL3 273-580, with catalytic residue (D395)=Italics

dCas13-M3nes:
(SEQ ID NO: 24)
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNAYKTYEEKLNDGCEFLTSTEQPLSGMINNYYTVALRNMNERY
GYKTEDLAFIQDKRFKFVKDAYGKKKSQVNTGFFLSLQDYNGDTQKKLHLSGVGIALLI
CLFLDKQYINIFLSRLPIFSSYNAQSEERRIIIRSFGINSIKLPKDRIHSEKSNKSVAMDMLN
EVKRCPDELFTTLSAEKQSRFRIISDDHNEVLMKRSSDRFVPLLLQYIDYGKLFDHIRFHV
NMGKLRYLLKADKTCIDGQTRVRVIEQPLNGFGRLEEAETMRKQENGTFGNSGIRIRDF
ENMKRDDANPANYPYIVDTYTHYILENNKVEMFINDKEDSAPLLPVIEDDRYVVKTIPSC
RMSTLEIPAMAFHMFLFGSKKTEKLIVDVHNRYKRLFQAMQKEEVTAENIASFGIAESD
LPQKILDLISGNAHGKDVDAFIRLTVDDMLTDTERRIKRFKDDRKSIRSADNKMGKRGF
KQISTGKLADFLAKDIVLFQPSVNDGENKITGLNYRIMQSAIAVYDSGDDYEAKQQFKL
MFEKARLIGKGTTEPHPFLYKVFARSIPANAVEFYERYLIERKFYLTGLSNEIKKGNRVD
VPFIRRDQNKWKTPAMKTLGRIYSEDLPVELPRQMFDNEIKSHLKSLPQMEGIDFNNAN
VTYLIAEYMKRVLDDDFQTFYQWNRNYRYMDMLKGEYDRKGSLQHCFTSVEEREGL
WKERASRTERYRKQASNKIRSNRQMRNASSEEIETILDKRLSNSRNEYQKSEKVIRRYRV
QDALLFLLAKKTLTELADFDGERFKLKEIMPDAEKGILSEIMPMSFTFEKGGKKYTITSE
GMKLKNYGDFFVLASDKRIGNLLELVGSDIVSKEDGSLQLPPLERLTLSGSETPGTSES
ATPESQEFCDYGTKEECMKASDADRPCRKLHFRRIINKHTDESLGDCSFLNTCFHMDTCKY
VHYEIDACMDSEAPGSKDHTPSQELALTQSVGGDSSADRLFPPQWICCDIRYLDVSILGKFAV
VMADPPWDIHMELPYGTLTDDEMRRLNIPVLQDDGFLFLWVTGRAMELGRECLNLWGYER
VDEIIWVKTNQLQRIIRTGRTGHWLNHGKEHCLVGVKGNPQGFNQGLDCDVIVAEVRSTSHK
PDEIYGMIERLSPGTRKIELFGRPHNVQPNWITLGNQLDGIHLLDPDVVARFKQRYPDGIISKP
KNL

HIV Nuclear export signal=Bold; SV40 Nuclear localization signal=Bold and Italics; dPspCas13b Δ984-1090=Plain Text; GS linker=Underlined; 16aa XTEN linker=Bold and Underlined; METTL3 273-580), with catalytic residue (D395)=Italics

dCas13-M3nls:
(SEQ ID NO: 25)
MKRTADGSEFESPKKKRKVNIPALVENQKKYFGTYSVMAMLNAQTVLDHIQKVADIEG
EQNENNENLWFHPVMSHLYNAKNGYDKQPEKTMFIIERLQSYFPFLKIMAENQREYSN
GKYKQNRVEVNSNDIFEVLKRAFGVLKMYRDLTNAYKTYEEKLNDGCEFLTSTEQPLS
GMINNYYTVALRNMNERYGYKTEDLAFIQDKRFKFVKDAYGKKKSQVNTGFFLSLQD
YNGDTQKKLHLSGVGIALLICLFLDKQYINIFLSRLPIFSSYNAQSEERRIIIRSFGINSIKLP
KDRIHSEKSNKSVAMDMLNEVKRCPDELFTTLSAEKQSRFRIISDDHNEVLMKRSSDRF
VPLLLQYIDYGKLFDHIRFHVNMGKLRYLLKADKTCIDGQTRVRVIEQPLNGFGRLEEA
ETMRKQENGTFGNSGIRIRDFENMKRDDANPANYPYIVDTYTHYILENNKVEMFINDKE
DSAPLLPVIEDDRYVVKTIPSCRMSTLEIPAMAFHMFLFGSKKTEKLIVDVHNRYKRLFQ
AMQKEEVTAENIASFGIAESDLPQKILDLISGNAHGKDVDAFIRLTVDDMLTDTERRIKR
FKDDRKSIRSADNKMGKRGFKQISTGKLADFLAKDIVLFQPSVNDGENKITGLNYRIMQ
SAIAVYDSGDDYEAKQQFKLMFEKARLIGKGTTEPHPFLYKVFARSIPANAVEFYERYLI
ERKFYLTGLSNEIKKGNRVDVPFIRRDQNKWKTPAMKTLGRIYSEDLPVELPRQMFDNE
IKSHLKSLPQMEGIDFNNANVTYLIAEYMKRVLDDDFQTFYQWNRNYRYMDMLKGEY
DRKGSLQHCFTSVEEREGLWKERASRTERYRKQASNKIRSNRQMRNASSEEIETILDKRL
SNSRNEYQKSEKVIRRYRVQDALLFLLAKKTLTELADFDGERFKLKEIMPDAEKGILSEI
MPMSFTFEKGGKKYTITSEGMKLKNYGDFFVLASDKRIGNLLELVGSDIVSKEDGSKRT
ADGSEFEPKKKRKVSGSETPGTSESATPESQEFCDYGTKEECMKASDADRPCRKLHFRRII
NKHTDESLGDCSFLNTCFHMDTCKYVHYEIDACMDSEAPGSKDHTPSQELALTQSVGGDSSA
DRLFPPQWICCDIRYLDVSILGKFAVVMADPPWDIHMELPYGTLTDDEMRRLNIPVLQDDGF
LFLWVTGRAMELGRECLNLWGYERVDEIIWVKTNQLQRIIRTGRTGHWLNHGKEHCLVGVK
GNPQGFNQGLDCDVIVAEVRSTSHKPDEIYGMIERLSPGTRKIELFGRPHNVQPNWITLGNQL
DGIHLLDPDVVARFKQRYPDGIISKPKNL

HIV Nuclear export signal=Bold; SV40 Nuclear localization signal=Bold and Italics; dPspCas13b Δ984-1090=Plain Text; GS linker=Underlined; 32aa GGS-XTEN-GGS linker=Bold and Underlined; METTL3 359-580, with catalytic residue (D395)=Italics; 30aa GGS linker between M3 and M14=Italics and Underlined; METTL14 111-456=Italics and Bold and Underlined

dCas13-M3M14nes:
(SEQ ID NO: 26)
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNAYKTYEEKLNDGCEFLTSTEQPLSGMINNYYTVALRNMNERY
GYKTEDLAFIQDKRFKFVKDAYGKKKSQVNTGFFLSLQDYNGDTQKKLHLSGVGIALLI
CLFLDKQYINIFLSRLPIFSSYNAQSEERRIIIRSFGINSIKLPKDRIHSEKSNKSVAMDMLN
EVKRCPDELFTTLSAEKQSRFRIISDDHNEVLMKRSSDRFVPLLLQYIDYGKLFDHIRFHV
NMGKLRYLLKADKTCIDGQTRVRVIEQPLNGFGRLEEAETMRKQENGTFGNSGIRIRDF
ENMKRDDANPANYPYIVDTYTHYILENNKVEMFINDKEDSAPLLPVIEDDRYVVKTIPSC
RMSTLEIPAMAFHMFLFGSKKTEKLIVDVHNRYKRLFQAMQKEEVTAENIASFGIAESD
LPQKILDLISGNAHGKDVDAFIRLTVDDMLTDTERRIKRFKDDRKSIRSADNKMGKRGF
KQISTGKLADFLAKDIVLFQPSVNDGENKITGLNYRIMQSAIAVYDSGDDYEAKQQFKL
MFEKARLIGKGTTEPHPFLYKVFARSIPANAVEFYERYLIERKFYLTGLSNEIKKGNRVD
VPFIRRDQNKWKTPAMKTLGRIYSEDLPVELPRQMFDNEIKSHLKSLPQMEGIDFNNAN
VTYLIAEYMKRVLDDDFQTFYQWNRNYRYMDMLKGEYDRKGSLQHCFTSVEEREGL
WKERASRTERYRKQASNKIRSNRQMRNASSEEIETILDKRLSNSRNEYQKSEKVIRRYRV
QDALLFLLAKKTLTELADFDGERFKLKEIMPDAEKGILSEIMPMSFTFEKGGKKYTITSE
GMKLKNYGDFFVLASDKRIGNLLELVGSDIVSKEDGSLQLPPLERLTLSGGSSGGSSGS
ETPGTSESATPESSGGSSGGSVGGDSSADRLFPPQWICCDIRYLDVSILGKFAVVMADPPW
DIHMELPYGTLTDDEMRRLNIPVLQDDGFLFLWVTGRAMELGRECLNLWGYERVDEIIWVKT
NQLQRIIRTGRTGHWLNHGKEHCLVGVKGNPQGFNQGLDCDVIVAEVRSTSHKPDEIYGMIE
RLSPGTRKIELFGRPHNVQPNWITLGNQLDGIHLLDPDVVARFKQRYPDGIISKPKNLGGSGG
SGGSGGSGGSGGSGGSGGSGGSGSGQSLNPHNDYCQHFVDTGHRPQNFIRDVGLADRFE
EYPKLRELIRLKDELIAKSNTPPMYLQADIEAFDIRELTPKFDVILLEPPLEEYYRETGITA
NEKCWTWDDIMKLEIDEIAAPRSFIFLWCGSGEGLDLGRVCLRKWGYRRCEDICWIKTN
KNNPGKTKTLDPKAVFQRTKEHCLMGIKGTVKRSTDGDFIHANVDIDLIITEEPEIGNIE
KPVEIFHIIEHFCLGRRRLHLFGRDSTIRPGWLTVGPTLTNSNYNAETYASYFSAPNSYLT
GCTEEIERLFPKSPPPKSKSDRGGGAPRGGGRGGTSAGRGRERNRSNFRGERGGFRGG
RGGAHRGGFPPR

HIV Nuclear export signal=Bold; SV40 Nuclear localization signal=Bold and Italics; dPspCas13b Δ984-1090=Plain Text; GS linker=Underlined; 32aa GGS-XTEN-GGS linker=Bold and Underlined; METTL3 359-580, with catalytic residue (D395)=Italics; 30aa GGS linker between M3 and M14=Italics and Underlined; METTL14 111-456=Italics and Bold and Underlined

dCas13-M3M14nls:
(SEQ ID NO: 27)
MKRTADGSEFESPKKKRKVNIPALVENQKKYFGTYSVMAMLNAQTVLDHIQKVADIEG
EQNENNENLWFHPVMSHLYNAKNGYDKQPEKTMFIIERLQSYFPFLKIMAENQREYSN
GKYKQNRVEVNSNDIFEVLKRAFGVLKMYRDLTNAYKTYEEKLNDGCEFLTSTEQPLS
GMINNYYTVALRNMNERYGYKTEDLAFIQDKRFKFVKDAYGKKKSQVNTGFFLSLQD
YNGDTQKKLHLSGVGIALLICLFLDKQYINIFLSRLPIFSSYNAQSEERRIIIRSFGINSIKLP
KDRIHSEKSNKSVAMDMLNEVKRCPDELFTTLSAEKQSRFRIISDDHNEVLMKRSSDRF
VPLLLQYIDYGKLFDHIRFHVNMGKLRYLLKADKTCIDGQTRVRVIEQPLNGFGRLEEA
ETMRKQENGTFGNSGIRIRDFENMKRDDANPANYPYIVDTYTHYILENNKVEMFINDKE
DSAPLLPVIEDDRYVVKTIPSCRMSTLEIPAMAFHMFLFGSKKTEKLIVDVHNRYKRLFQ
AMQKEEVTAENIASFGIAESDLPQKILDLISGNAHGKDVDAFIRLTVDDMLTDTERRIKR
FKDDRKSIRSADNKMGKRGFKQISTGKLADFLAKDIVLFQPSVNDGENKITGLNYRIMQ
SAIAVYDSGDDYEAKQQFKLMFEKARLIGKGTTEPHPFLYKVFARSIPANAVEFYERYLI
ERKFYLTGLSNEIKKGNRVDVPFIRRDQNKWKTPAMKTLGRIYSEDLPVELPRQMFDNE
IKSHLKSLPQMEGIDFNNANVTYLIAEYMKRVLDDDFQTFYQWNRNYRYMDMLKGEY
DRKGSLQHCFTSVEEREGLWKERASRTERYRKQASNKIRSNRQMRNASSEEIETILDKRL
SNSRNEYQKSEKVIRRYRVQDALLFLLAKKTLTELADFDGERFKLKEIMPDAEKGILSEI
MPMSFTFEKGGKKYTITSEGMKLKNYGDFFVLASDKRIGNLLELVGSDIVSKEDGSKRT
ADGSEFEPKKKRKVSGGSSGGSSGSETPGTSESATPESSGGSSGGSVGGDSSADRLFPP
QWICCDIRYLDVSILGKFAVVMADPPWDIHMELPYGTLTDDEMRRLNIPVLQDDGFLFLWVT
GRAMELGRECLNLWGYERVDEIIWVKTNQLQRIIRTGRTGHWLNHGKEHCLVGVKGNPQGF
NQGLDCDVIVAEVRSTSHKPDEIYGMIERLSPGTRKIELFGRPHNVQPNWITLGNQLDGIHLL
DPDVVARFKQRYPDGIISKPKNLGGSGGSGGSGGSGGSGGSGGSGGSGGSGSGQSQSLNPHND
YCQHFVDTGHRPQNFIRDVGLADRFEEYPKLRELIRLKDELIAKSNTPPMYLQADIEAF
DIRELTPKFDVILLEPPLEEYYRETGITANEKCWTWDDIMKLEIDEIAAPRSFIFLWCGS
GEGLDLGRVCLRKWGYRRCEDICWIKTNKNNPGKTKTLDPKAVFQRTKEHCLMGIKGT
VKRSTDGDHIHANVDIDLIITEEPEIGNIEKPVEIFHIIEHFCLGRRRLHLFGRDSTIRPG
WLTVGPTLTNSNYNAETYASYFSAPNSYLTGCTEEIERLRPKSPPPKSKSDRGGGAPRGG
GRGGTSAGRGRERNRSNFRGERGGFRGGRGGAHRGGFPPR

### REFERENCES (CITED IN EXAMPLE 2)

1. Goldberg, A. D., Allis, C. D. & Bernstein, E. Epigenetics: a landscape takes shape. Cell 128, 635-638 (2007).
2. Pawson, T. & Scott, J. D. Protein phosphorylation in signaling—50 years and counting. Trends in biochemical sciences 30, 286-290 (2005).
3. Pickart, C. M. & Eddins, M. J. Ubiquitin: structures, functions, mechanisms. Biochimica et biophysica acta 1695, 55-72 (2004).
4. Hoernes, T. P. & Erlacher, M. D. Translating the epitranscriptome. Wiley Interdiscip Rev RNA 8 (2017).
5. Meyer, K. D. & Jaffrey, S. R. The dynamic epitranscriptome: N6-methyladenosine and gene expression control. Nature reviews. Molecular cell biology 15, 313-326 (2014).
6. Engel, M. & Chen, A. The emerging role of mRNA methylation in normal and pathological behavior. Genes Brain Behav (2017).
7. Roundtree, I. A. et al. YTHDC1 mediates nuclear export of N(6)-methyladenosine methylated mRNAs. Elife 6 (2017).
8. Shi, H. et al. YTHDF3 facilitates translation and decay of N(6)-methyladenosine-modified RNA. Cell Res 27, 315-328 (2017).
9. Xiao, W. et al. Nuclear m⁶A Reader YTHDC1 Regulates mRNA Splicing. Molecular Cell 61, 507-519 (2016).
10. Wang, X. et al. N(6)-methyladenosine Modulates Messenger RNA Translation Efficiency. Cell 161, 1388-1399 (2015).
11. Wang, X. et al. N6-methyladenosine-dependent regulation of messenger RNA stability. Nature 505, 117-120 (2014).
12. Meyer, K. D. et al. Comprehensive analysis of mRNA methylation reveals enrichment in 3′ UTRs and near stop codons. Cell 149, 1635-1646 (2012).
13. Dominissini, D. et al. Topology of the human and mouse m⁶A RNA methylomes revealed by m⁶A-seq. Nature 485, 201-206 (2012).
14. Choi, J. et al. N(6)-methyladenosine in mRNA disrupts tRNA selection and translation-elongation dynamics. Nature structural & molecular biology 23, 110-115 (2016).
15. Zhou, J. et al. Dynamic m(6)A mRNA methylation directs translational control of heat shock response. Nature 526, 591-594 (2015).
16. Liu, N. et al. N(6)-methyladenosine-dependent RNA structural switches regulate RNA-protein interactions. Nature 518, 560-564 (2015).
17. Alarcon, C. R., Lee, H., Goodarzi, H., Halberg, N. & Tavazoie, S. F. N6-methyladenosine marks primary microRNAs for processing. Nature 519, 482-485 (2015).
18. Aguilo, F. et al. Coordination of m(6)A mRNA Methylation and Gene Transcription by ZFP217 Regulates Pluripotency and Reprogramming. Cell stem cell 17, 689-704 (2015).
19. Chen, T. et al. m(6)A RNA methylation is regulated by microRNAs and promotes reprogramming to pluripotency. Cell stem cell 16, 289-301 (2015).
20. Xiang, Y. et al. RNA m⁶A methylation regulates the ultraviolet-induced DNA damage response. Nature 543, 573-576 (2017).
21. Jaffrey, S. R. & Kharas, M. G. Emerging links between m⁶A and misregulated mRNA methylation in cancer. Genome medicine 9, 2 (2017).
22. Cui, Q. et al. m⁶A RNA Methylation Regulates the Self-Renewal and Tumorigenesis of Glioblastoma Stem Cells. Cell reports 18, 2622-2634 (2017).
23. Liu, J. et al. A METTL3-METTL14 complex mediates mammalian nuclear RNA N6-adenosine methylation. Nat Chem Biol 10, 93-95 (2014).
24. Sledz, P. & Jinek, M. Structural insights into the molecular mechanism of the m(6)A writer complex. Elife 5 (2016).
25. Scholler, E. et al. Interactions, localization, and phosphorylation of the m(6)A generating METTL3-METTL14-WTAP complex. RNA 24, 499-512 (2018).
26. Bartosovic, M. et al. N6-methyladenosine demethylase FTO targets pre-mRNAs and regulates alternative splicing and 3′-end processing. Nucleic acids research 45, 11356-11370 (2017).
27. Zheng, G. et al. ALKBH5 is a mammalian RNA demethylase that impacts RNA metabolism and mouse fertility. Mol Cell 49, 18-29 (2013).
28. Wang, X. & He, C. Reading RNA methylation codes through methyl-specific binding proteins. RNA Biol 11, 669-672 (2014).
29. Alarcon, C. R. et al. HNRNPA2B1 Is a Mediator of m(6)A-Dependent Nuclear RNA Processing Events. Cell 162, 1299-1308 (2015).
30. Wu, B., Li, L., Huang, Y., Ma, J. & Min, J. Readers, writers and erasers of N(6)-methylated adenosine modification. Curr Opin Struct Biol 47, 67-76 (2017).
31. Abudayyeh, O. O. et al. RNA targeting with CRISPR-Cas13. Nature 550, 280-284 (2017).
32. Cox, D. B. T. et al. RNA editing with CRISPR-Cas13. Science (2017).
33. Wang, X. et al. Structural basis of N(6)-adenosine methylation by the METTL3-METTL14 complex. Nature 534, 575-578 (2016).
34. Li, F. et al. A Radioactivity-Based Assay for Screening Human m⁶A-RNA Methyltransferase, METTL3-METTL14 Complex, and Demethylase ALKBH5. Journal of biomolecular screening 21, 290-297 (2016).
35. Liu, L. et al. The Molecular Architecture for RNA-Guided RNA Cleavage by Cas13a. Cell 170, 714-726 e710 (2017).
36. Knott, G. J. et al. Guide-bound structures of an RNA-targeting A-cleaving CRISPR-Cas13a enzyme. Nature structural & molecular biology 24, 825-833 (2017).
37. Liu, N. et al. Probing N6-methyladenosine RNA modification status at single nucleotide resolution in mRNA and long noncoding RNA. RNA 19, 1848-1856 (2013).
38. Imanishi, M., Tsuji, S., Suda, A. & Futaki, S. Detection of N(6)-methyladenosine based on the methyl-sensitivity of MazF RNA endonuclease. Chemical communications 53, 12930-12933 (2017).
39. Crick, F. H. On protein synthesis. Symp Soc Exp Biol 12, 138-163 (1958).

### OTHER EMBODIMENTS

The foregoing has been a description of certain non-limiting embodiments of the disclosure. Those of ordinary skill in the art will appreciate that various changes and modifications to this description may be made without departing from the spirit or scope of the present disclosure, as defined in the following claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A fusion protein comprising an RNA programmable RNA binding domain (RNApRNAbd), a METTL3 methyltransferase or a fragment thereof, and one or more nuclear localization sequences (NLS).

2. The fusion protein of claim 1, wherein the RNApRNAbd is a Type VI CRISPR-Cas protein.

3. The fusion protein of claim 1, wherein the RNApRNAbd comprises an amino acid sequence that is at least 80% identical to the amino acid sequence of SEQ ID NO: 1 or 2.

4. The fusion protein of claim 1, wherein the METTL3 methyltransferase comprises an amino acid sequence that is at least 80% identical to the amino acid sequence of SEQ ID NO: 3 or a fragment thereof.

5. The fusion protein of claim 1, wherein the fusion protein comprises the structure: NH₂-[NLS]-[RNApRNAbd]-[NLS]-[METTL3 or fragment thereof]-COOH, wherein each instance of “]-[” is an optional linker.

6. The fusion protein of claim 1, wherein the fusion protein is capable of making an N⁶-methyladenosine (m⁶A) modification in an RNA in the nucleus of a cell.

7. A complex comprising the fusion protein of claim 1 and a guide RNA (gRNA) bound to the RNApRNAbd of the fusion protein.

8. A method comprising contacting an RNA molecule with the fusion protein of claim 1.

9. A polynucleotide encoding the fusion protein of claim 1.

10. A cell comprising the fusion protein of claim 1.

11. A fusion protein comprising an RNA programmable RNA binding domain (RNApRNAbd), a METTL3 methyltransferase or a fragment thereof, a METTL14 methyltransferase or a fragment thereof, and one or more nuclear export sequences (NES).

12. The fusion protein of claim 11, wherein the RNApRNAbd is a Type VI CRISPR-Cas protein.

13. The fusion protein of claim 11, wherein the RNApRNAbd comprises an amino acid sequence that is at least 80% identical to the amino acid sequence of SEQ ID NO: 1 or 2.

14. The fusion protein of claim 11, wherein the METTL3 methyltransferase comprises an amino acid sequence that is at least 80% identical to the amino acid sequence of SEQ ID NO: 3 or a fragment thereof, and wherein the METTL14 methyltransferase comprises an amino acid sequence that is at least 80% identical to the amino acid sequence of SEQ ID NO: 4 or a fragment thereof.

15. The fusion protein of claim 11, wherein the fusion protein comprises the structure: NH₂-[RNAPRNAbd]-[NES]-[METTL3 or fragment thereof]-[METTL14 or fragment thereof]-COOH, wherein each instance of “]-[” comprises an optional linker.

16. The fusion protein of claim 11, wherein the effector domain is capable of making an N⁶-methyladenosine (m⁶A) modification in an RNA in the cytoplasm of a cell.

17. A complex comprising the fusion protein of claim 11 and a guide RNA (gRNA) bound to the RNApRNAbd of the fusion protein.

18. A method comprising contacting an RNA molecule with the fusion protein of claim 11.

19. A polynucleotide encoding the fusion protein of claim 11.

20. A cell comprising the fusion protein of claim 11.


SEQUENCE LISTING (38 sequences)

SEQ ID NO: 1 - protein, 983 aa, synthetic construct
  REGION 1..983: note = Synthetic polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKED

SEQ ID NO: 2 - protein, 967 aa, synthetic construct
  REGION 1..967: note = Synthetic polypeptide
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 3 - protein, 580 aa, synthetic construct
  REGION 1..580: note = Synthetic polypeptide
MSDTWSSIQAHKKQLDSLRERLQRRRKQDSGHLDLRNPEAALSPTFRSDSPVPTAPTSGGPKPSTASAVPELATDPELEKKLLHHLSDLALTLPTDAVSICLAISTPDAPATQDGVESLLQKFAAQELIEVKRGLLQDDAHPTLVTYADHSKLSAMMGAVAEKKGPGEVAGTVTGQKRRAEQDSTTVAAFASSLVSGLNSSASEPAKEPAKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 4 - protein, 456 aa, synthetic construct
  REGION 1..456: note = Synthetic polypeptide
MDSRLQEIRERQKLRRQLLAQQLGAESADSIGAVLNSKDEQREIAETRETCRASYDTSAPNAKRKYLDEGETDEDKMEEYKDELEMQQDEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 5 - protein, 349 aa, synthetic construct
  REGION 1..349: note = Synthetic polypeptide
MLNTVKISSCELINADCLEFIRSLPENSVDLIVTDPPYFKVKPEGWDNQWKGDDDYLKWLDQCLAQFWRVLKPAGSLYLFCGHRLASDIEIMMRERFSVLNHIIWAKPSGRWNGCNKESLRAYFPATERILFAEHYQGPYRPKDAGYEAKGRALKQHVMAPLIAYFRDARAALGITAKQIADATGKKNMVPHWFSASQWQLPNESDYLKLQSLFARVAEEKHQRGELEKPHHQLVSTYSELNRKYMELLSEYKNLRRYFGVTVQVPYTDVWTYKPVQYYPGKHPCEKPAEMLQQIISASSRPGDLVADFFMGSGSTVKAAMALGRRAIGVELETGRFEQTVREVQDLIV

SEQ ID NO: 6 - protein, 280 aa, synthetic construct
  REGION 1..280: note = Synthetic polypeptide
MSATGPFSIGERVQLTDAKGRRYTMSLTPGAEFHTHRGSIAHDAVIGLEQGSVVKSSNGALFLVLRPLLVDYVMSMPRGPQVIYPKDAAQIVHEGDIFPGARVLEAGAGSGALTLSLLRAVGPAGQVISYEQRADHAEHARRNVSGCYGQPPDNWRLVVSDLADSELPDGSVDRAVLDMLAPWEVLDAVSRLLVAGGVLMVYVATVTQLSRIVEALRAKQCWTEPRAWETLQRGWNVVGLAVRPQHSMRGHTAFLVATRRLAPGAVAPAPLGRKREGRDG

SEQ ID NO: 7 - protein, 477 aa, synthetic construct [sequence omitted]

SEQ ID NO: 8 - protein, 767 aa, synthetic construct
  REGION 1..767: note = Synthetic polypeptide
MGRRSRGRRLQQQQRPEDAEDGAEGGGKRGEAGWEGGYPEIVKENKLFEHYYQELKIVPEGEWGQFMDALREPLPATLRITGYKSHAKEILHCLKNKYFKELEDLEVDGQKVEVPQPLSWYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIEDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCGWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 9 - protein, 391 aa, synthetic construct
  REGION 1..391: note = Synthetic polypeptide
MEPLRVLELYSGVGGMHHALRESCIPAQVVAAIDVNTVANEVYKYNFPHTQLLAKTIEGITLEEFDRLSFDMILMSPPCQPFTRIGRQGDMTDSRTNSFLHILDILPRLQKLPKYILLENVKGFEVSSTRDLLIQTIENCGFQYQEFLLSPTSLGIPNSRLRYFLIAKLQSEPLPFQAPGQVLMEFPKIESVHPQKYAMDVENKIQEKNVEPNISFDGSIQCSGKDAILFKLETAEEIHRKNQQDSDLSVKMLKDFLEDDTDVNQYLLPPKSLLRYALLLDIVQPTCRRSVCFTKGYGSYIEGTGSVLQTAEDVQVENIYKSLTNLSQEEQITKLLILKLRYFTPKEIANLLGFPPEFGFPEKITVKQRYRLLGNSLNVHVVAKLIKILYE

SEQ ID NO: 10 - protein, 396 aa, synthetic construct
  REGION 1..396: note = Synthetic polypeptide
MSVRLVLAKGREKSLLRRHPWVFSGAVARMEGKASLGETIDIVDHQGKWLARGAYSPASQIRARVWTFDPSESIDIAFFSRRLQQAQKWRDWLAQKDGLDSYRLIAGESDGLPGITIDRFGNFLVLQLLSAGAEYQRAALISALQTLYPECSIYDRSDVAVRKKEGMELTQGPVTGELPPALLPIEEHGMKLLVDIQHGHKTGYYLDQRDSRLATRRYVENKRVLNCFSYTGGFAVSALMGGCSQVVSVDTSQEALDIARQNVELNKLDLSKAEFVRDDVFKLLRTYRDRGEKFDVIVMDPPKFVENKSQLMGACRGYKDINMLAIQLLNEGGILLTFSCSGLMTSDLFQKIIADAAIDAGRDVQFIEQFRQAADHPVIATYPEGLYLKGFACRVM

SEQ ID NO: 11 - protein, 394 aa, synthetic construct
  REGION 1..394: note = Synthetic polypeptide
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHRPRILEMDKEENRRSVLLPTHRRRGSFSSENYWRKSYESSEDCSEAAGSPARKVKMRRH

SEQ ID NO: 12 - protein, 505 aa, synthetic construct
  REGION 1..505: note = Synthetic polypeptide
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 13 - protein, 150 aa, synthetic construct
  REGION 1..150: note = Synthetic polypeptide
  VARIANT 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 14 - protein, 150 aa, synthetic construct
  REGION 1..150: note = Synthetic polypeptide
  VARIANT 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 15 - protein, 120 aa, synthetic construct
  REGION 1..120: note = Synthetic polypeptide
  VARIANT 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 16 - protein, 16 aa, synthetic construct
  REGION 1..16: note = Synthetic polypeptide
SGSETPGTSESATPES

SEQ ID NO: 17 - protein, 7 aa, synthetic construct
  REGION 1..7: note = Synthetic polypeptide
PKKKRKV

SEQ ID NO: 18 - protein, 30 aa, synthetic construct
  REGION 1..30: note = Synthetic polypeptide
MDSLLMNRRKFLYQFKNVRWAKGRRETYLC

SEQ ID NO: 19 - protein, 18 aa, synthetic construct
  REGION 1..18: note = Synthetic polypeptide
KRTADGSEFESPKKKRKV

SEQ ID NO: 20 - protein, 17 aa, synthetic construct
  REGION 1..17: note = Synthetic polypeptide
KRTADGSEFEPKKKRKV

SEQ ID NO: 21 - protein, 16 aa, unidentified
  REGION 1..16: note = Xenopus nucleoplasmin
  VARIANT 3..12: note = X can be any naturally occurring amino acid
KRXXXXXXXXXXKKKL

SEQ ID NO: 22 - protein, 8 aa, synthetic construct
  REGION 1..8: note = Synthetic polypeptide
SGGSSGGS

SEQ ID NO: 23 - protein, 32 aa, synthetic construct
  REGION 1..32: note = Synthetic polypeptide
SGGSSGGSSGSETPGTSESATPESSGGSSGGS

SEQ ID NO: 24 - protein, 1320 aa, synthetic construct
  REGION 1..1320: note = Synthetic polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLSGSETPGTSESATPESQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 25 - protein, 1344 aa, synthetic construct
  REGION 1..1344: note = Synthetic polypeptide
MKRTADGSEFESPKKKRKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSKRTADGSEFEPKKKRKVSGSETPGTSESATPESQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 26 - protein, 1626 aa, synthetic construct
  REGION 1..1626: note = Synthetic polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLSGGSSGGSSGSETPGTSESATPESSGGSSGGSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNLGGSGGSGGSGGSGGSGGSGGSGGSGGSGSGQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 27 - protein, 1650 aa, synthetic construct
  REGION 1..1650: note = Synthetic polypeptide
MKRTADGSEFESPKKKRKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSKRTADGSEFEPKKKRKVSGGSSGGSSGSETPGTSESATPESSGGSSGGSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNLGGSGGSGGSGGSGGSGGSGGSGGSGGSGSGQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 28 - protein, 27 aa, synthetic construct
  REGION 1..27: note = Synthetic polypeptide
YPYDVPDYAYPYDVPDYAYPYDVPDYA

SEQ ID NO: 29 - DNA, 30 bp, synthetic construct
  misc_feature 1..30: note = Synthetic polynucleotide
  source 1..30: mol_type = other DNA
gtaatgcctggcttgtcgacgcatagtctg

SEQ ID NO: 30 - DNA, 30 bp, synthetic construct
  misc_feature 1..30: note = Synthetic polynucleotide
  source 1..30: mol_type = other DNA
ttccaaactatcctgcggcctctactctgc

SEQ ID NO: 31 - DNA, 30 bp, synthetic construct
  misc_feature 1..30: note = Synthetic polynucleotide
  source 1..30: mol_type = other DNA
tacatagctgcattcggagatactctatgt

SEQ ID NO: 32 - DNA, 30 bp, synthetic construct
  misc_feature 1..30: note = Synthetic polynucleotide
  source 1..30: mol_type = other DNA
gaagcatttgcggtggacgatggaggggcc

SEQ ID NO: 33 - DNA, 30 bp, synthetic construct
  misc_feature 1..30: note = Synthetic polynucleotide
  source 1..30: mol_type = other DNA
agccccgcggccatcacgccacagtttccc

SEQ ID NO: 34 - protein, 11 aa, synthetic construct
  REGION 1..11: note = Synthetic polypeptide
LQLPPLERLTL

SEQ ID NO: 35 - protein, 30 aa, synthetic construct
  REGION 1..30: note = Synthetic polypeptide
  VARIANT 2..30: note = may be absent
GGGGGGGGGGGGGGGGGGGGGGGGGGGGGG

SEQ ID NO: 36 - protein, 90 aa, synthetic construct
  REGION 1..90: note = Synthetic polypeptide
  VARIANT 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 37 - protein, 60 aa, synthetic construct
  REGION 1..60: note = Synthetic polypeptide
  VARIANT 1: note = X can be any naturally occurring amino acid
  VARIANT 3: note = X can be any naturally occurring amino acid
  VARIANT 5: note = X can be any naturally occurring amino acid
  VARIANT 7: note = X can be any naturally occurring amino acid
  VARIANT 9: note = X can be any naturally occurring amino acid
  VARIANT 11: note = X can be any naturally occurring amino acid
  VARIANT 13: note = X can be any naturally occurring amino acid
  VARIANT 15: note = X can be any naturally occurring amino acid
  VARIANT 17: note = X can be any naturally occurring amino acid
  VARIANT 19: note = X can be any naturally occurring amino acid
  VARIANT 21: note = X can be any naturally occurring amino acid
  VARIANT 23: note = X can be any naturally occurring amino acid
  VARIANT 25: note = X can be any naturally occurring amino acid
  VARIANT 27: note = X can be any naturally occurring amino acid
  VARIANT 29: note = X can be any naturally occurring amino acid
  VARIANT 31: note = X can be any naturally occurring amino acid
  VARIANT 33: note = X can be any naturally occurring amino acid
  VARIANT 35: note = X can be any naturally occurring amino acid
  VARIANT 37: note = X can be any naturally occurring amino acid
  VARIANT 39: note = X can be any naturally occurring amino acid
  VARIANT 41: note = X can be any naturally occurring amino acid
  VARIANT 43: note = X can be any naturally occurring amino acid
  VARIANT 45: note = X can be any naturally occurring amino acid
  VARIANT 47: note = X can be any naturally occurring amino acid
  VARIANT 49: note = X can be any naturally occurring amino acid
  VARIANT 51: note = X can be any naturally occurring amino acid
  VARIANT 53: note = X can be any naturally occurring amino acid
  VARIANT 55: note = X can be any naturally occurring amino acid
  VARIANT 57: note = X can be any naturally occurring amino acid
  VARIANT 59: note = X can be any naturally occurring amino acid
  VARIANT 3..60: note = may be absent
XPXPXPXPXPXPXPXPXPXPXPXPXPXPXPXPXPXPXPXPXPXPXPXPXPXPXPXPXPXP

SEQ ID NO: 38 - protein, 21 aa, synthetic construct
  REGION 1..21: note = Synthetic polypeptide
  VARIANT 4..21: note = may be absent
GGSGGSGGSGGSGGSGGSGGS